(19) United States
(12) Patent Application Publication
(10) Pub. No.: US 2022/0362405 A1
(43) Pub. Date: Nov. 17, 2022

(54) COMPOSITIONS AND METHODS FOR TREATING LIVER DISEASE

(71) Applicant: UNIVERSITY OF PITTSBURGH-OF THE COMMONWEALTH SYSTEM OF HIGHER EDUCATION, Pittsburgh, PA (US)

(72) Inventors: Alejandro Soto Gutierrez, Pittsburgh, PA (US); Aaron Bell, West Mifflin, PA (US); Nicolas Fraunhoffer Navarro, Pittsburgh, PA (US); Jorge Guzman Lepe, Pittsburgh, PA (US); Sarah Hainer, Pittsburgh, PA (US); George K. Michalopoulos, Pittsburgh, PA (US); Alina Ostrowska, Mckees Rocks, PA (US); Ira Fox, Pittsburgh, PA (US); Edgar Naoe Tafaleng, Pittsburgh, PA (US); Kazuki Takeishi, Fukuoka (JP)

(21) Appl. No.: 17/769,886

(22) PCT Filed: Oct. 14, 2020
(86) PCT No.: PCT/US2020/055500

§ 371 Date: Apr. 18, 2022

Related U.S. Application Data
(60) Provisional application No. 62/915,765, filed on Oct. 16, 2019.

(51) Int. Cl. A61K 48/00; A61P 1/16

(52) U.S. Cl. CPC A61K 48/005; A61P 1/16

(57) ABSTRACT

Disclosed are compositions and methods for treating a liver disease in a subject by increasing transport or retention of HNF4α, a transcriptional factor, into a nucleus of a hepatocyte in the subject. In some embodiments, the method comprises upregulating expression or function of one or more transcription factors selected from the group consisting of PROX1, NR5A2, NROB2, MTF1, SREBP1, EP300, and POM121C, and functional fragments thereof, and/or downregulating expression or function of one or more transcription factors DNAJB 1/F1SP40, ATF6, ATF4, and PERK, and functional fragments thereof.

### CROSS-REFERENCE TO RELATED APPLICATIONS

This application claims the priority benefit of U.S. Provisional Application No. 62/915,765, filed Oct. 16, 2019, which is incorporated herein by reference in its entirety.

### STATEMENT REGARDING FEDERALLY SPONSORED RESEARCH OR DEVELOPMENT

This invention was made with government support under grant number DK099257 awarded by the National Institutes of Health. The government has certain rights in the invention.

### FIELD

The present disclosure relates to modulation of HNF4α expression or activity to, e.g., treat liver disease and/or liver disorders.

### BACKGROUND

Terminal liver failure (TLF) as a consequence of advanced liver cirrhosis, represented the twelfth leading cause of death in 2015 with an estimated 15.8 deaths per 100,000 population globally (Tsochatzis E A et al., 2014). In the United States, the number of registered deaths coupled to chronic liver disease and cirrhosis in 2015 was 40,326, with a mortality of 12.5 deaths per 100,000 population, an interannual increase of 3.8% (Murphy S L et al., 2017; Goldman L, et al., 2016). The most affected age range is among 45 to 64 years old with a mortality of 26.4 deaths per 100,000 population, which ranks chronic liver disease and cirrhosis as the fourth leading cause of death in this age range after cancer, heart disease, and unintentional injuries (Murphy S L et al., 2017). The only definitive therapy for TLF is orthotopic liver transplantation which, given the number of patients in need of liver transplants and the inadequate number of available organs, essentially makes TLF an untreatable disease (Lopez P M et al., 2006).

There are numerous causes of chronic liver disease, including chronic infection by hepatitis viruses, alcohol-mediated cirrhosis, and non-alcoholic steatohepatitis (NASH) (Archambeaud I et al., 2015; Donato F et al,. 2006; Gelatti U et at, 2005; Kuper H et al., 2000, and each can produce hepatocellular failure (Guzman-Lepe J et al., 2018; Hernaez R et al., 2017; Lee Y A et al., 2015; Pessayre D et al., 1978). The mechanisms responsible for deterioration of hepatocyte function and ultimately hepatic failure in man are poorly understood.

The principal causes of chronic liver disease, cirrhosis, and lately TLF are related to infection by hepatitis B and C viruses, alcohol-mediated Laennec's cirrhosis, and non-alcoholic steatohepatitis (NASH)/metabolic syndrome (Archambeaud et al., 2015; Donato F et al., 2006; Gelatti et al., 2005; Kuper et al., 2000). These etiologic agents cause fibrosis that disrupts the normal lobular architecture with alterations of the vasculature (Goldman L, et al., 2016). These pathological changes have been associated with hepatocellular failure and inability of hepatocytes to perform their normal functions (Guzman-Lepe J et al., 2018; Hernaez R et al., 2007; Lee Ya et al., 2015; Pessayre D et al., 1978) but the mechanisms responsible for deterioration of hepatocyte function and ultimately hepatic failure are unknown in humans. The chronic hepatic damage produces oxidative stress (Cichoz-Lach H et al., 2014; Simoes ICM et al., 2018) and endoplasmic reticulum stress (Malhi H et al., 2011; Zhang X Q et al., 2014) that induce cell death (Cichoz-Lach H et al., 2014; Malhi H et al., 2011; Zhang X Q et al., 2014; Wang K et al., 2014; Seki E et al., 2015) and ultimately reducing the proliferative capacity of the hepatocytes (Zhang BH et al., 1999; Michalopoulos GK et al., 2015; Dubuquoy L et al., 2015).

It has been determined that liver-enriched transcription factors are stably down regulated in hepatocytes from rats with end-stage cirrhosis (Nishikawa T et al., 2014; Guzman-Lepe J et al., 2019), and that forced re-expression of one of them, hepatocyte nuclear factor 4 alpha (HNF4α), reprograms dysfunctional hepatocytes to regain function, both in culture and in vivo (Nishikawa T et al., 2014). A study of a large cohort of patients with advanced liver disease shows that the level of HNF4α mRNA expression in the diseased liver correlated with the extent of hepatic dysfunction (Childs-Pugh classification) and that HNF4α expression was not localized in the nucleus (Guzman-Lepe J et al., 2019). Forced re-expression of hepatocyte nuclear factor 4 alpha (HNF4α) can reprogram dysfunctional hepatocytes from terminally cirrhotic livers to function again, both in culture and in vivo, without the expansion of new hepatocytes or stem cells (Nishikawa T et al., 2014). Down-regulation of LETFs, including a significant reduction in HNF4α nuclear localization and mRNA expression, is associated with the extent of hepatic dysfunction in a large cohort of human livers with TLF (Guzman-Lepe J et al., 2018).

HNF4α is a transcription factor that plays a critical role in liver organogenesis and hepatocyte function in adult livers (Nishikawa T et al., 2014; Babeu J P et al., 2014). The main HNF4α action has been regulation of specifically targeted genes involved in lipid, glucose, xenobiotic, and drug metabolism (Nishikawa T et al., 2014; Babeu J P et al., 2014). A single gene codes for HNF4α in human (Kritis A A et al., 1999), which is regulated by two different promoters. These promoters produce two isoform classes, P1 and P2 (Babeu J P et al., 2014). P1 isoforms are mainly expressed in the adult liver, whereas P2 isoforms have been detected in the liver during embryonic development and under pathological conditions such as cancer (Babeu J P et al., 2014; Walesky C et al., 2015; Tanaka T et al., 2006). The expression and function of HNF4α are regulated at multiple levels (Chellappa K et al., 2012; Guo H et al., 2014; Hong YH et al., 2003; Lu H et al., 2016, Song Y et al., 2015; Soutoglou E et al., 2000; Sun K et al., 2007; Xu Z et al., 2007; Yokoyama A et al., 2011; Zhou W et al., 2012).

Therefore, what is needed are compositions and methods for modulating HNF4α expression and/or treating liver diseases and/or liver disorders. The compositions and methods disclosed herein address these and other needs.

### SUMMARY

The compositions and methods disclosed herein address the certain unmet needs in liver disease treatment. In some aspects, disclosed herein are compositions and uses thereof for a medicament for the treatment of liver diseases and/or liver disorders, wherein the composition increases an amount or function of one or more transcription factors selected from the group consisting of PROX1, NR5A2, NR0B2, MTF1, SREBP1, EP300, and/or POM121C or decreases an amount or suppresses a function of one or more transcription factors selected from the group consisting of DNAJB1/HSP40, ATF6, ATF4, and PERK. In some embodiments, the composition is a vector, and wherein the vector comprises one or more nucleic acids that encode the one or more of PROX1, NR5A2, NR0B2, MTF1, SREBP1, EP300, and/or POM121C. In some embodiments, the composition is a vector comprising a nucleic acid encoding HNF4α (e.g., HNF4α isoform 2). The compositions and the methods disclosed herein result in surprising increase in an amount of HNF4α in a hepatocyte (e.g., an increase in a total amount of HNF4α in the hepatocyte, and/or an increase in an amount of HNF4α in the nucleus of the hepatocyte), resulting in an effective treatment of liver diseases (e.g., an end-stage liver disease).

In some aspects, disclosed herein is a method of treating a liver disease in a subject in need thereof comprising administering to the subject a composition, wherein the composition increases an amount or function of one or more transcription factors selected from the group consisting of PROX1, NR5A2, NR0B2, MTF1, SREBP1, EP300, and POM121C.

In some aspects, disclosed herein is a use of a composition for the preparation of a medicament for treatment a liver disease in a subject in need thereof comprising administering to the subject the composition, wherein the composition increases an amount or function of one or more transcription factors selected from the group consisting of PROX1, NR5A2, NR0B2, MTF1, SREBP1, EP300, and POM121C.

In some embodiments, the composition is a vector, and wherein the vector comprises one or more nucleic acids that encode the one or more of PROX1, NR5A2, NR0B2, MTF1, SREBP1, EP300, and POM121C. In some embodiments, the vector comprises one or more nucleic acids that encode the PROX1 and/or SREBP1. The one or more nucleic acids can be a DNA or a mRNA.

In some aspects, disclosed herein is a method of treating a liver disease in a subject in need thereof comprising administering to the subject a composition, wherein the composition decreases an amount or suppresses a function of one or more transcription factors selected from the group consisting of DNAJB1/HSP40, ATF6, ATF4, and PERK.

In some aspects, disclosed herein is a use of a composition for the preparation of a medicament for treatment a liver disease in a subject in need thereof comprising administering to the subject the composition, wherein the composition decreases an amount or function of one or more transcription factors selected from the group consisting of DNAJB1/HSP40, ATF6, ATF4, and PERK.

In some embodiments, the administration of the composition increases an amount of HNF4α in a nucleus of a hepatocyte in the subject. In some embodiments, the administration of the composition does not increase a total amount of HNF4α in the hepatocyte. In some embodiments, the administration of the composition increases a total amount of HNF4α in the hepatocyte.

In some embodiments, the vector further comprises a nucleic acid that encodes HNF4α. In some embodiments, the method further comprises administering to the subject a vector that comprises a nucleic acid that encodes HNF4α. In one example, the nucleic acid encodes HNF4α isoform 2.

In some aspects, disclosed herein is a composition comprising a vector, wherein the vector comprises one or more nucleic acids that encode one or more transcription factors selected from the group consisting of PROX1, NR5A2, NR0B2, MTF1, SREBP1, EP300, and POM121C, and functional fragments thereof.

In some aspects, disclosed herein is a method of treating a liver disease in a subject in need thereof comprising administering to the subject a vector that comprises a nucleic acid encoding HNF4α isoform 2.

In some aspects, disclosed herein is a use of a vector for preparation for medicament for treatment of a liver disease, wherein the vector comprises a nucleic acid encoding HNF4α isoform 2.

### DESCRIPTION OF DRAWINGS

FIGS. 1A-1C show HNF4α location in hepatocytes from normal and cirrhotic livers. FIG. 1A shows representative photographs of HNF4α immunofluorescence of isolated human hepatocytes from NASH decompensated liver and normal human hepatocytes. Western blot analysis and quantification of HNF4α normalized to Beta-Actin in hepatocytes isolated from functionally decompensated livers (NASH and alcohol-mediated Laennec's cirrhosis) (n=6) and hepatocytes isolated from normal liver controls (n=2). FIG. 1A shows total HNF4α; normal human hepatocytes vs decompensated human hepatocytes, P=0.166. FIG. 1B shows cytoplasm HNF4α; normal human hepatocytes vs decompensated human hepatocytes, P=0.023. FIG. 1C shows nuclear HNF4α; normal human hepatocytes vs decompensated human hepatocytes, P=0.023). Graphs from A to C are plotted as the mean±SD. Statistically significant (P<0.05). Diamonds show Child-Pugh “B” and squares show Child-Pugh “C”.

FIGS. 2A-2C show protein expression and Spearman's rank correlation test of HNF4α post translational modifiers. FIGS. 2A and 2B show Western blot analysis (2A) and quantification (2B) of EGFR (P=0.904), cMET (P=0.023), total AMPKα (P>0.999), p-AMPKα(Thr172) (P=0.547), total AKT (P=0.047), p-AKT(Ser473) (P=0.547), p-AKT(Thr308) (P=0.024), p-AMPKα(Thr172)/AMPKα ratio (P=0.5476), p-AKT(Ser473)/AKT ratio (P=0.1667) and p-AKT(Thr308)/total AKT ratio (P=0.0238) in decompensated NASH (n=4) and alcohol-mediated Laennec's cirrhotic hepatocytes (n=2) and normal control hepatocytes (n=2). In FIG. 2B, filled squares refer to Child-Pugh “B” and filled diamonds refer to Child-Pugh “C”. FIG. 2C shows that Spearman's rank correlation test is shown and demonstrates that nuclear HNF4α correlates significantly with cMET (r=0.71; P=0.037), total AKT (r=0.71; P=0.037), phospho-AKT(Thr308) (r=0.82; P=0.011), and phospho-AKT(Thr308)/total AKT ratio (r=0.73; P=0.031). Cytoplasmic HNF4α showed significant correlation with cMET (r=−0.80; P=0.014), total AKT (r=−0.73; P=0.031), phospho-AKT(Thr308) (r=−0.77; P=0.021, and phospho-AKT(Thr308)/total AKT ratio (r=−0.72; P=0.037). Bar graphs of FIG. 2B are plotted as the mean±SD. Statistically significant (P<0.05).

FIGS. 3A-3D show the relationship of post-translational modifiers and HNF4α cellular localization. FIG. 3A shows that path analysis revealed a significant direct relation between HNF4α localization with cMET (0.56; P=0.004), phosphoAKT(Thr308)/Total AKT ratio (0.05; P=0.006) and total HNF4α levels (0.60; P=0.042). Path analysis revealed a negative significant relationship between cMET and total HNF4α (−0.37; P=0.024). FIG. 3B depicts that linear regression analysis showed the significant relation of nuclear HNF4α expression and the degree of liver dysfunction (Child-Pugh score) (R²⁼0.80, P=0.007). FIG. 3C depicts that principal component analysis (PCA) showed a protein profile that correlates with HNF4α expression explain positively the characteristics of normal human hepatocytes (n=2) while cytoplasmic HNF4α, active caspase 3, p-AKT(Ser473)/total AKT ratio and p-AMPK(Ser172)/AMPK explained the characteristics of decompensated human hepatocytes from NASH (n=4) and alcohol-mediated Laennec's cirrhotic livers (n=2). Graphs of FIG. 3D show protein expression fold changes used for PCA analysis of total, nucleus, or cytoplasmic HNF4α (the three graphs in the top row), cMET, p-AMPK(Ser172)/AMPK, p-AKT(Ser473)/total AKT, p-AKT(Ser473)/total AKT and phosphoAKT(Thr308)/Total AKT ratio, p-H3(Ser10) and active Caspase (graphs in the second and bottom rows) in decompensated human hepatocytes (Child-Pugh Classification B and C) relative to normal human hepatocytes. The graphs are plotted as the mean±SD. Statistically significant (P<0.05).

FIGS. 4A-4C show that acetylation of nuclear HNF4α is altered in human decompensated hepatocytes from NASH and alcohol-mediated Laennec's cirrhotic explanted livers. FIGS. 4A and 4B show Western blots and quantification of the acetylated form of HNF4α (Lys106) in the nuclear fraction of human hepatocytes from decompensated NASH (n=4) and alcohol-mediated Laennec's cirrhotic (n=4) explanted livers (P=0.024). The linear regression analysis in FIG. 4C shows a significant correlation of reduced acetylated form of HNF4α (Lys106) and liver dysfunction (R²=0.71, P=0.004). Bar graph is plotted as the mean±SD. Statistically significant (P<0.05).

FIG. 5 shows in silico analysis of HNF4α-post-translational modifications (PTMs). FIG. 5 provides a list of the HNF4α-PTMs.

FIGS. 6A-6B show the relationship of the activated AKT pathway with HNF4α post-translational modifiers and p-EGFR expression in human decompensatedhepatocytes from NASH and alcohol-mediated Laennec's cirrhotic explanted livers. FIG. 6A shows Spearman correlation for phospho-AKT (Thr308)/AKT. FIG. 6B shows Spearman correlation for phospho-AKT (Ser473)/AKT.

FIGS. 7A-7C show the expression levels of the liver-enriched transcription factor HNF4α a in human hepatocytes isolated from an explanted liver of a patient with alcoholic hepatitis at the mRNA level (FIG. 7A) compared to that of freshly isolated normal human hepatocytes and at a protein level (FIG. 7B) by immunohistochemistry demonstrating that only about 40% of the alcohol hepatitis hepatocytes express HNF4α in the nuclei with a weak intensity and about 10% of the cells had HNF4α cytoplasmic expression. For FIG. 7C, freshly isolated human alcohol hepatitis hepatocytes were treated with a lentivirus encoding HNF4α(Systems Bioscience, Cat #CS970S-1; HNF4α in CD511B-1). This figure shows that after 72 h, HNF4α expression did not change the percent of hepatocytes expressing HNF4α in the nuclei. However, HNF4α expression intensity increased dramatically in the existing cells. Altogether this data show that HNF4α transportation to the nucleus can play an important role for the hepatocytes to regain function in humans with alcoholic hepatitis.

FIGS. 8A-8F show MTF1 expression in primary human hepatocytes. FIG. 8A shows that primary human hepatocytes isolated from livers of patients undergoing liver transplantation for NASH or Alcohol-induced cirrhosis were analyzed for the expression of MTF1 (MA5-26738 1:1000) and HNF4α (ab41898 1:1000) by Western Blot. FIGS. 8B and 8C show the relative intensity of HNF4α (FIGS. 8B and 8C) and MTF1 (FIGS. 8D and 8E) among the control hepatocytes, Child B hepatocytes and Child C hepatocytes was compared by One-Way ANOVA with Brown-Forsythe and Welch ANOVA Test for multiple comparisons. The expression of HNF4α and MTF1 is lower in Child C hepatocytes compared to Child B hepatocytes. FIGS. 8B and 8D, *p<0.003, **p<0.01, ***p<0.0001, n=25. FIG. 8C R²=0.019, p=0.06, n=19. FIG. 8E R²=0.015, p=0.1, n=19. FIG. 8F shows that correlation studies with Child-Pugh Score, protein expression of HNF4α and MTF1 were performed using Simple linear regression. Black circles refer to control, light grey circles refer to Child B, dark grey circles refer to Child C. The protein expression of HNF4α correlates with the protein expression of MTF1 (R²=0.28, p=0.007, n=25).

FIGS. 9A-9D show NR0B2 expression in primary human hepatocytes. FIG. 9A shows that primary human hepatocytes isolated from livers of patients undergoing liver transplantation for NASH or Alcohol-induced cirrhosis were analyzed for the expression of NR0B2 (Abclonal A1836 1:500) and HNF4α (ab41898 1:1000) by Western Blot. FIGS. 9B and 9C show the relative intensity of NR0B2 among the control hepatocytes, Child B hepatocytes and Child C hepatocytes was compared by One-Way ANOVA with Brown-Forsythe and Welch ANOVA Test for multiple comparisons. FIG. 9B *p<0.05, **p<0.01, ***p<0.001, n=25. FIG. 9C R²=0.19, p=0.06, n=19. The expression of NR0B2 is different in Child C, Child B and Control hepatocytes. FIG. 9D shows that correlation studies with Child-Pugh Score, protein expression of HNF4α and NR0B2 were performed using Simple linear regression. Black circles refer to control, light grey circles refer to Child B, dark grey circles refer to Child C (R²=0.12, p=0.1, n=25).

FIGS. 10A-10D show NR5A2 expression in primary human hepatocytes. FIG. 10A shows that primary human hepatocytes isolated from livers of patients undergoing liver transplantation for NASH or Alcohol-induced cirrhosis were analyzed for the expression of NR5A2 (Novus NBP2-27196 1:500) and HNF4α (ab41898 1:1000) by Western Blot. FIGS. 10B and 10C show the relative intensity of NR5A2 among the control hepatocytes, Child B hepatocytes and Child C hepatocytes was compared by One-Way ANOVA with Brown-Forsythe and Welch ANOVA Test for multiple comparisons. FIG. 10B *p<0.05, n=25. FIG. 10C R²=0.17, p=0.07, n=19. The expression of NR5A2 is different between Child B and Child C and Control hepatocytes. FIG. 10B shows that correlation studies with Child-Pugh Score, protein expression of HNF4α and NR0B2 were performed using Simple linear regression. The protein expression of NRSA2 correlates with the expression of HNF4α. Black circles refer to control, light grey circles refer to Child B, dark grey circles refer to Child C (R²=0.17, p<0.05, n=25).

FIGS. 11A-11D show Proxl expression in primary human hepatocytes. FIG. 11A shows that primary human hepatocytes isolated from livers of patients undergoing liver transplantation for NASH or Alcohol-induced cirrhosis were analyzed for the expression of PROX1 (R&D AF2727 1:500) and HNF4α (ab41898 1:1000) by Western Blot. FIGS. 11B and 11C show the relative intensity of PROX1 among the control hepatocytes, Child B hepatocytes and Child C hepatocytes compared by One-Way ANOVA with Brown-Forsythe and Welch ANOVA Test for multiple comparisons. FIG. 11B *p<0.02, n=25. FIG. 11C R²=0.02, p=0.6, n=19. The expression of PROX1 is different in Child C and Control hepatocytes. FIG. 11D shows that correlation studies with Child-Pugh Score, protein expression of HNF4α and PROX1 were performed using Simple linear regression. Black circles refer to control, light grey circles refer to Child B, dark grey circles refer to Child C (R²=0.02, p=0.46, n=25).

FIGS. 12A-12D show POM121C expression in primary human hepatocytes. FIG. 12A shows that primary human hepatocytes isolated from livers of patients undergoing liver transplantation for NASH or Alcohol-induced cirrhosis were analyzed for the expression of POM121C (PAS-85161 1:500) and HNF4α (ab41898 1:1000) by Western Blot. FIGS. 12B and 12C show the relative intensity of POM121C among the control hepatocytes, Child B hepatocytes and Child C hepatocytes was compared by One-Way ANOVA with Brown-Forsythe and Welch ANOVA Test for multiple comparisons. FIG. 12B n=25. FIG. 12C R²=0.08, p=0.24, n=25. FIG. 12D shows that correlation studies with Child-Pugh Score, protein expression of HNF4α and POM121C were performed using Simple linear regression. Black circles refer to control, light grey circles refer to Child B, dark grey circles refer to Child C (R²=0.06, p=0.23, n=25).

FIGS. 13A-13D show SREBP1 expression in primary human hepatocytes. FIG. 13A shows that primary human hepatocytes isolated from livers of patients undergoing liver transplantation for NASH or Alcohol-induced cirrhosis were analyzed for the expression of SREBP1 (Abcam ab28481 1:500) and HNF4α (ab41898 1:1000) by Western Blot. FIGS. 13B and 13C show the relative intensity of SREBP1 among the control hepatocytes, Child B hepatocytes and Child C hepatocytes was compared by One-Way ANOVA with Brown-Forsythe and Welch ANOVA Test for multiple comparisons. FIG. 13B n=25. FIG. 13C R²=0.02, p=0.54, n=19. FIG. 13D shows that correlation studies with Child-Pugh Score, protein expression of HNF4α and SREBP1 were performed using Simple linear regression. Black circles refer to control, light grey circles refer to Child B, dark grey circles refer to Child C (R²=0.01, p=0.86, n=25).

FIGS. 14A-14D show EP300 expression in primary human hepatocytes. FIG. 14A shows that primary human hepatocytes isolated from livers of patients undergoing liver transplantation for NASH or Alcohol-induced cirrhosis were analyzed for the expression of EP300 (Novus NB100-616 1:500) and HNF4α (ab41898 1:1000) by Western Blot. FIGS. 14B and 14C show the relative intensity of EP300 among the control hepatocytes, Child B hepatocytes and Child C hepatocytes was compared by One-Way ANOVA with Brown-Forsythe and Welch ANOVA Test for multiple comparisons. FIG. 14B n=25. FIG. 14C R²=0.32, p=0.01, n=19. FIG. 14D shows that correlation studies with Child-Pugh Score, protein expression of HNF4α and EP300 were performed using Simple linear regression. Black circles refer to control, light grey circles refer to Child B, dark grey circles refer to Child C (R²=0.01, p=0.69, n=25).

FIGS. 15A and B show that CRISPR/Cas9 Knockout of EP300, MTF1, NR0B2, NR5A2, POM121C, PROX1 or SREBP1 in HepG2 cells was performed and the cellular HNF4α location was analyzed in immunofluorescence (ab41898 1:500). The total number of nuclei positive in DAPI and HNF4α (FIG. 15A) and cells positive for HNF4α in the cytoplasm (FIG. 15B) were counted. The statistical analysis was performed using One-Way ANOVA with Brown-Forsythe and Welch ANOVA Test for multiple comparisons. Knockout of EP300, MTF1, NR0B2, NR5A2, POM121C, PROX1 and SREBP1 showed a reduced nuclear location of HNF4α and an increase in cytoplasmic location of HNF4α. *p<0.05.

FIG. 16 shows that primary human hepatocytes isolated from a patient with NASH undergoing liver transplantation were transduced with AAV- HNF4α and AAV-MTF1, NR0B2, NR5A2, POM121C, PROX1, SREBP1 or GFP with a MOI of 10⁵. The percentage of nuclei positive for HNF4α was counted. The statistical analysis was performed using One-Way ANOVA with Brown-Forsythe and Welch ANOVA Test for multiple comparisons. Co-Transduction with HNF4α, MTF1, NR0B2, POM121C, PROX1 and SREBP1 lead to an increase in the number of positive nuclei compared to the GFPHNF4α co-transduction group (****p<0.0001, ***p<0.0005, **p<0.001, *p<0.05).

### DETAILED DESCRIPTION

Disclosed herein are compositions and methods for treating liver disease in a subject by increasing the expression and/or the transport or retention of HNF4α, a transcriptional factor, into a nucleus of a hepatocyte in the subject. In some embodiments, the method comprises upregulating expression or function of one or more transcription factors selected from the group consisting of PROX1, NR5A2, NR0B2, MTF1, SREBP1, EP300, and POM121C, and functional fragments thereof, and/or downregulating expression or function of one or more transcription factors DNAJB1/HSP40, ATF6, ATF4, and PERK, and functional fragments thereof. It is a surprising finding that these transcription factors modulate expression and/or localization of HNF4α, and therefore, can be used for the treatment of liver disease.

In some embodiments, the method comprises administering a vector, wherein the vector comprises a nucleic acid (e.g., DNA, ceDNA, or mRNA) that encodes one or more transcriptional factors selected from the group consisting of PROX1, NR5A2, NR0B2, MTF1, SREBP1, EP300, and POM121C, and functional fragments thereof. In some aspects, the method comprises administering vector comprising a nucleic acid (e.g., DNA, ceDNA or mRNA) that encodes HNF4α (e.g., HNF4α isoform 2). In other or further embodiments, the method comprises administering a composition, wherein the composition decreases an amount or suppresses function of one or more transcription factors selected from the group consisting of DNAJB1/HSP40, ATF6, ATF4, and PERK, and functional fragments thereof. In other embodiments, the method comprises increasing acetylation of HNF4α at Lys106, increasing expression of cMET, and/or increasing activation of AKT via phosphorylation at Thr308. The methods disclosed herein have been shown to surprisingly increase an amount of HNF4α in a nucleus of a hepatocyte. Such manipulation of HNF4α improves hepatocyte function in patients with liver disease.

Terms used throughout this application are to be construed with ordinary and typical meaning to those of ordinary skill in the art. However, Applicants desire that the following terms be given the particular definition as provided below.

### Terminology

As used in the specification and claims, the singular form “a,” “an,” and “the” include plural references unless the context clearly dictates otherwise. For example, the term “a cell” includes a plurality of cells, including mixtures thereof.

The term “about” as used herein when referring to a measurable value such as an amount, a percentage, and the like, is meant to encompass variations of ±20%, ±10%, ±5%, or ±1% from the measurable value.

“Administration” to a subject or “administering” includes any route of introducing or delivering to a subject an agent. Administration can be carried out by any suitable route, including intravenous, intraperitoneal, and the like. Administration includes self-administration and the administration by another.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a liver disease). The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc. In some aspects, the composition disclosed herein comprises a vector, wherein the vector comprises a nucleic acid that encodes one or more transcription factors selected from the group consisting of PROX1, NR5A2, NR0B2, MTF1, SREBP1, EP300, and POM121C, and functional fragments thereof. In some aspects, the composition disclosed herein comprises a nucleic acid which decreases an amount or suppresses function of one or more transcription factors selected from the group consisting of DNAJB1/HSP40, ATF6, ATF4, and PERK, and functional fragments thereof. In some aspects, the composition disclosed herein comprises a vector, wherein the vector comprises a nucleic acid that encodes HNFα.

“Effective amount” encompasses, without limitation, an amount that can ameliorate, reverse, mitigate, prevent, or diagnose a symptom or sign of a medical condition or disorder (e.g., a liver disease). Unless dictated otherwise, explicitly or by context, an “effective amount” is not limited to a minimal amount sufficient to ameliorate a condition. The severity of a disease or disorder, as well as the ability of a treatment to prevent, treat, or mitigate, the disease or disorder can be measured, without implying any limitation, by a biomarker or by a clinical parameter. The term “effective amount of a vector” or “effective amount of a composition” refers to an amount of a vector or a composition sufficient to cause some mitigation of a liver disease or restoration of liver function.

The “fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the fragment must possess a bioactive property, such as regulating the transcription of the target gene.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site).

“Liver disease” as used herein refers generally to diseases, disorders, and conditions affecting the liver, and may have a wide range of severity encompassing, for example, simple accumulation of fat in the hepatocytes (steatosis), nonalcoholic steatohepatitis (NASH), nonalcoholic fatty liver disease (NAFLD), alcohol liver disease (ALD), alcohol-related liver disease (including, but not limited to fatty liver, alcoholic hepatitis, alcohol-related cirrhosis), macrovesicular steatosis, periportal and lobular inflammation (steatohepatitis), cirrhosis, fibrosis, liver ischemia, liver cancer including hepatocellular carcinoma, hepatitis A, hepatitis B, hepatitis C, idiopathic liver disease, end-stage liver disease, and liver failure. “Liver cirrhosis” is defined herein as a chronic disease of the liver marked by a fibrous thickening of the liver tissue and/or regenerative nodules. The degree or severity of “liver cirrhosis” can be designated by a Child-Pugh score wherein five clinical measures, levels of total bilirubin, serum albumin, prothrombin time prolongation, ascites, and hepatic encephalopathy, are scored using a point system of 1 point, 2 point, and 3 point values for varying levels of each clinical measure, with 3 point values being assigned to the most severe levels of each measure. The total points for all five measures are added to arrive at a Child-Pugh score and classification. Scores of 5-6 designate Child-Pugh Class A, scores of 7-9 designate Child-Pugh Class B, and scores of 10-15 designate Child-Pugh Class C. In general, Child-Pugh Class A indicates the least severe liver disease and Child-Pugh Class C indicates the most severe liver disease. Accordingly, in some embodiments, the method disclosed herein can be used to treat a subject having a Child-Pugh Class B or Child-Pugh Class C liver disease. In some embodiments, the method disclosed here in can be used to treat a subject having a Child-Pugh Class A liver disease. In various aspects, the method improves the Child-Pugh score of the subject. In some embodiments, the liver disease is alcoholic hepatitis. In some embodiments, the method disclosed here in can be used to treat an ischemic donor liver for ex vivo perfusion. The present invention can be used to treat liver cancer before or after cancer treatment including before or after liver resection.

The term “nucleic acid” as used herein means a polymer composed of nucleotides, e.g. deoxyribonucleotides (DNA) or ribonucleotides (RNA). The terms “ribonucleic acid” and “RNA” as used herein mean a polymer composed of ribonucleotides. The terms “deoxyribonucleic acid” and “DNA” as used herein mean a polymer composed of deoxyribonucleotides. In some embodiments, the nucleic acid is DNA (e.g., ceDNA or cDNA). In some embodiments, the nucleic acid is mRNA.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The term “promoter” or “regulatory element” refers to a region or sequence determinants located upstream or downstream from the start of transcription and which are involved in recognition and binding of RNA polymerase and other proteins to initiate transcription. Promoters need not be of bacterial origin, for example, promoters derived from viruses or from other organisms can be used in the compositions, systems, or methods described herein.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, Pa., 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, N.J.), polyethylene glycol (PEG), and PLURONICS™ (BASF; Florham Park, N.J.). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

The term “subject” is defined herein to include animals such as mammals, including, but not limited to, primates (e.g., humans), cows, sheep, goats, horses, dogs, cats, rabbits, rats, mice and the like. In some embodiments, the subject is a human

The term “transcription factor” used herein refers to a protein that is involved in the process of transcribing DNA to RNA. Typically, a transcription factor possesses a domain that binds to the promoter or enhancer region of a specific gene. A transcription factor can also possess a domain that interacts with RNA polymerase and/or some other transcription factors, such interactions consequently regulates the amount of RNA transcribed from a DNA. Transcription factors can reside in the cytoplasm and be translocated to the nucleus upon activation.

The terms “treat,” “treating,” “treatment,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the invention may be applied preventively, prophylactically, palliatively or remedially. Treatments are administered to a subject prior to onset (e.g., before obvious signs of a liver disease), during early onset (e.g., upon initial signs and symptoms of a liver disease), after an established development of a liver disease, or at the stage of terminal liver failure. Prophylactic administration can occur for several days to years prior to the manifestation of symptoms of a liver disease.

In some instances, the terms “treat,” “treating,” “treatment” and grammatical variations thereof, include mitigating a liver disease, restoring liver function, and/or increasing the amount of HNFα in a nucleus of a hepatocyte in a subject, as compared with prior to treatment of the subject or as compared with incidence of such symptom in a general or study population.

“Vector” as used herein is a composition of matter which comprises an isolated nucleic acid and which can be used to deliver the isolated nucleic acid to the interior of a cell. A vector may be either a self-replicating, extrachromosomal vector or a vector which integrates into a host genome. Alternatively, a vector may also be a vehicle comprising the aforementioned nucleic acid sequence. A vector may be a plasmid, bacteriophage, viral vector (isolated, attenuated, recombinant, encapsulated as a viral particle, etc.), liposome, exosome, extracellular vesicle, microparticle and/or a nanoparticle. A vector may comprise a double-stranded or single-stranded DNA, RNA, or hybrid DNA/RNA sequence comprising double-stranded and/or single-stranded nucleotides. In some embodiments, the vector is a viral vector that comprises a nucleic acid sequence that is a viral packaging sequence responsible for packaging one or a plurality of nucleic acid sequences that encode one or a plurality of polypeptides. In some embodiments, the vector is a plasmid. In some embodiments the vector is an exosome. In some embodiments, the vector is a viral particle. In some embodiments, the viral particle is a lentivirus particle. In some embodiments, the vector is viral vector with a natural and/or an engineered capsid. In some embodiments, the vector comprises a viral particle comprising a nucleic acid sequence operably linked to a regulatory sequence, wherein the nucleic acid sequence encodes a fusion protein comprising one or a plurality of AAV viral particle polypeptides or fragments thereof. In some embodiments, the vector is a nanoparticle comprising a nucleic acid or a polypeptide. In various embodiments, the vector is a lipid-based nanoparticle.

### Compositions

As noted above, disclosed herein are methods of treating a liver disease by increasing the expression and/or transport and/or retention of HNF4α, a transcriptional factor, into a nucleus of a hepatocyte in the subject. In some embodiments, the method comprises upregulating expression or function of one or more transcription factors selected from the group consisting of PROX1, NR5A2, NR0B2, MTF1, SREBP1, EP300, and POM121C, and/or downregulating expression or function of one or more transcription factors selected from the group consisting of DNAJB1/HSP40, ATF6, ATF4, and PERK. In some embodiments, the method further comprises upregulating expression or function of HNF4α inside a cell (e.g., a liver cell), preferably inside the nucleus of the cell, optionally by increasing expression of endogenous HNFα or by introducing exogenous HNFα. It is described herein that upregulation of PROX1, NR5A2, NR0B2, MTF1, SREBP1, EP300, and POM121C and/or downregulation of DNAJB1/HSP40, ATF6, ATF4, and PERK increases levels of HNF4α in a nucleus of a hepatocyte, leading to restoration of liver function and mitigation of liver disease. In other embodiments, the method comprises increasing acetylation of HNF4α at Lys106, increasing expression of cMET, and/or increasing activation of AKT via phosphorylation at Thr308. These methods can be used in conjunction with the methods described in U.S. Patent Application Publication 2014/0249209, which is incorporated by reference in its entirety.

In some embodiments, the method further comprises upregulating expression or function of HNF4α together with one or more transcription factors selected from the group consisting of PROX1, NR5A2, NR0B2, MTF1, SREBP1, EP300, and POM121C. In some embodiments, the composition further comprises upregulating expression or function of HNF4α and PROX1. In some embodiments, the method further comprises upregulating expression or function of HNF4α and SREBP1. In some embodiments, the method further comprises upregulating expression or function of HNF4α, PROX1, and SREBP1.

Therefore, included herein are compositions that increase expression or function of one or more transcription factors selected from the group consisting of PROX1, NR5A2, NR0B2, MTF1, SREBP1, EP300, and POM121C and/or that decrease expression or function of one or more transcription factors selected from the group consisting of DNAJB1/HSP40, ATF6, ATF4, and PERK. In some embodiments, the composition upregulates expression or function of HNF4α together with one or more transcription factors selected from the group consisting of PROX1, NR5A2, NR0B2, MTF1, SREBP1, EP300, and POM121C. In some embodiments, the composition upregulates expression or function of HNF4α and PROX1. In some embodiments, the composition upregulates expression or function of HNF4α and SREBP1. In some embodiments, the composition upregulates expression or function of HNF4α, PROX1, and SREBP1.

Disclosed herein is a composition comprising a vector, wherein the vector comprises a nucleic acid that encodes one or more transcription factors selected from the group consisting of PROX1, NR5A2, NR0B2, MTF1, SREBP1, EP300, and/or POM121C, and functional fragments thereof. In some embodiments, the vector comprises a nucleic acid that encodes PROX1 or a functional fragment thereof. In other embodiments, the vector comprises a nucleic acid that encodes NR5A2 or a functional fragment thereof. In other embodiments, the vector comprises a nucleic acid that encodes NR0B2 or a functional fragment thereof. In other embodiments, the vector comprises a nucleic acid that encodes MTF1 or a functional fragment thereof. In other embodiments, the vector comprises a nucleic acid that encodes SREBP1 or a functional fragment thereof. In other embodiments, the vector comprises a nucleic acid that encodes EP300 or a functional fragment thereof. In other embodiments, the vector comprises a nucleic acid that encodes POM121C or a functional fragment thereof. In some embodiments, the vector further comprises a nucleic acid that encodes HNF4α. In some embodiments, the vector comprises a nucleic acid that encodes PROX1 and SREBP1. In some embodiments, the vector comprises a nucleic acid that encodes HNF4α, PROX1, and SREBP1. In some embodiments, the vector comprises a nucleic acid that encodes HNF4α and PROX1. In some embodiments, the vector comprises a nucleic acid that encodes HNF4α and SREBP1.

Besides the liver, HNF4α is also highly expressed in the kidney, small intestine, colon, and pancreas, where it also plays important roles. Polymorphic mutations of the HNF4α gene are associated with a wide spectrum of diseases, including maturity-onset diabetes of the young (MODY), Crohn's disease, and inflammatory bowel syndrome. Transcription from P1 or P2 promoters combined with alternative splicing can generate 12 different transcripts. Relative isoform expression is tissue dependent. The 12 isoforms differ only at N and C termini, which are responsible for activating and repressing transcription, respectively (see Ko et al., Cell Rep. 2019 Mar. 5; 26(10):2549-2557.e3, incorporated by reference herein in its entirety). It is increasingly recognized that each isoform performs a distinct function to regulate a specific subset of genes in a tissue-dependent manner For example, HNF4α isoform 2 is reportedly enriched in liver and acts as a tumor suppressor whose loss is associated with hepatocarcinoma or liver failure as described in this application, whereas HNF4 α isoform 8 is highly expressed in colon and controls the expression of growth-promoting genes.

Accordingly, in some embodiments, the vector disclosed herein further comprises a nucleic acid encoding an HNF4α isoform 2 polypeptide. In some embodiments, the HNF4α isoform 2 polypeptide comprises a sequence at least about 80%, about 85%, about 90%, about 95%, or about 98% identical to SEQ ID NO: 1 or a fragment thereof. In some embodiments, the nucleic acid at least about 80%, about 85%, about 90%, about 95%, or about 98% identical to SEQ ID NO: 31 or a fragment thereof. In some aspects, the HNF4α isoform 2 polypeptide is promoter 1 (P1) driven, or in other words, its expression is driven by a P1 promoter of HNF4α. This is designated herein as HNF4α isoform 2 (P1). Accordingly, in some embodiments, the HNF4α isoform 2 polynucleotide or nucleic acid is operably linked to a P1 promoter.

A vector can be a nucleic acid sequence comprising a regulatory nucleic acid sequence that controls the replication of an expressible gene. In some embodiments, a vector comprising a promoter operably linked to a second nucleic acid (e.g., polynucleotide encoding a transcription factor) may include a promoter that is heterologous to the second nucleic acid (e.g., polynucleotide encoding a transcription factor) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). It should be understood herein that the vector of any aspects described herein can further comprise a promoter, an enhancer, an antibiotic resistance gene, and/or an origin, which can be operably linked to one or more of the above noted transcription factors.

In some embodiments, the vector can be a viral vector. “Viral vector” as disclosed herein means, in respect to a vehicle, any virus, virus-like particle, virion, viral particle, or pseudotyped virus that comprises a nucleic acid sequence that directs packaging of a nucleic acid sequence in the virus, virus-like particle, virion, viral particle, or pseudotyped virus. In some embodiments, the virus, virus-like particle, virion, viral particle, or pseudotyped virus is capable of transferring a vector (such as a nucleic acid vector) into and/or between host cells. In some embodiments, the virus, virus-like particle, virion, viral particle, or pseudotyped virus is capable of transferring a vector (such as a nucleic acid vector) into and/or between target cells, such as a hepatocyte in the liver of a subject. Importantly, in some embodiments, the virus, virus-like particle, virion, viral particle, or pseudotyped virus is capable of transporting into a nucleus of a target cell (e.g., a hepatocyte). The term “viral vector” is also meant to refer to those forms described more fully in U.S. Patent Application Publication U.S. 2018/0057839, which is incorporated herein by reference for all purposes. Suitable viral vectors include, e.g., adenoviruses, adeno-associated virus (AAV), vaccinia viruses, herpesviruses, baculoviruses and retroviruses, parvoviruses, and lentiviruses. In some embodiments, the viral vector is a lentiviral vector or an adeno-associated viral vector.

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

Another type of viral vector is based on an adeno-associated virus (AAV). This defective parvovirus can infect many cell types and is nonpathogenic to humans. AAV type vectors can transport about 4 to 5 kb and wild type AAV is known to stably insert into chromosome 19. The AAV inverted terminal repeats (ITRs), or modifications thereof, confer infectivity and site-specific integration, but not cytotoxicity, and the promoter directs cell-specific expression. U.S. Pat. No. 6,261,834 is herein incorporated by reference for material related to the AAV vector. The methods of using AAV vectors for transducing liver cells in vivo are known in the art. See U.S. Pat. No. 9,981,048, incorporated by reference herein in its entirety.

Viral vectors, especially adenoviral vectors can be complexed with a cationic amphiphile, such as a cationic lipid, polyL-lysine (PLL), and diethylaminoethyldextran (DELAE-dextran), which provide increased efficiency of viral infection of target cells (See, e.g., PCT/US97/21496 filed Nov. 20, 1997, incorporated herein by reference). AAV vectors, such as those disclosed in Zhong et al., J. Genet Syndr Gene Therapy 2012 Jan. 10; S1. pii: 008, U.S. Pat. Nos. 5,139,941, 5,252,479 and 5,753,500 and PCT publication WO 97/09441, the disclosures of which are incorporated herein, are also useful since these vectors integrate into host chromosomes, with a minimal need for repeat administration of vector. For a review of viral vectors in gene therapy, see McConnell et al., 2004, Hum Gene Ther. 15(11):1022-33; Mccarty et al., 2004, Annu Rev Genet. 38:819-45; Mah et al., 2002, Clin. Pharmacokinet. 41(12):901-11; Scott et al., 2002, Neuromuscul. Disord. 12(Suppl 1):S23-9.

In some embodiments, the vector is a nanoparticle. The nanoparticle used herein can be any nanoparticle useful for the delivery of nucleic acids. The term “nanoparticle” as used herein refers to a particle or structure which is biocompatible with and sufficiently resistant to chemical and/or physical destruction by the environment of such use so that a sufficient number of the nanoparticles remain substantially intact after delivery to the site of application or treatment and whose size is in the nanometer range. In some embodiments, the nanoparticle comprises a lipid-like nanoparticle. See, for example, WO WO2016/187531A1, WO2017/176974, WO2019/027999, or Li, B et al., An Orthogonal array optimization of lipid-like nanoparticles for mRNA delivery in vivo. Nano Lett. 2015, 15, 8099-8107; which are incorporated herein by reference. In some embodiments, the nanoparticle can comprise a lipid bilayer or liposome. In some embodiments, the vector is an mRNA lipid nanoparticle.

In some embodiments, the disclosed nanoparticles may be able to efficiently bind to or otherwise associate with a biological entity, for example, a particular membrane component or cell surface receptor on a target cell (e.g., a receptor that facilitates delivery into a liver cell or a receptor on a liver cell). For example, the disclosed nanoparticles may be engineered to include a ligand that binds to a receptor expressed on a normal or a diseased liver cell (e.g., a hepatic asialoglycoprotein receptor (ASGPR) or low density lipoprotein (LDLR) receptor).

In some aspects, the nanoparticle disclosed herein can comprise a supplemental component that facilitates delivery of the nucleic acid into a liver cell. The nanoparticle can comprise a cationic lipid, a helper lipid, cholesterol, and polyethylene glycol (PEG). In some embodiments, the nanoparticle comprises 5A2-SC8, 1,2-dioleoyl-snglycero-3-phosphoethanolamine (DOPE), cholesterol, and/or 1,2-dimyristoyl-rac-glycerol-methoxy(poly(ethylene glycol)), or any combination thereof. In some embodiments, the nanoparticle further comprises 5A2-SC8, 1,2-dioleoyl-snglycero-3-phosphoethanolamine (DOPE), cholesterol, and 1,2-dimyristoyl-rac-glycerol-methoxy(poly(ethylene glycol)). In some embodiments, the nanoparticle further comprises 1,2-dioleoyl-3-trimethylammonium-propane (DOTAP). In some embodiments, the molar ratio of 5A2-SC8, 1,2-dioleoyl-snglycero-3-phosphoethanolamine (DOPE), cholesterol, and 1,2-dimyristoyl-rac-glycerol-methoxy(poly(ethylene glycol)) of the nanoparticle is about 15/15/30/3.

In some embodiments, the nanoparticle comprises DLin-MC3-DMA, 1,2-distearoyl-sn-glycero-3-phosphocholine (DSPC), cholesterol, and 1,2-dimyristoyl-rac-glycerol-methoxy(poly(ethylene glycol)). In some embodiments, the molar ratio of DLin-MC3-DMA, 1,2-distearoyl-sn-glycero-3-phosphocholine (DSPC), cholesterol, and 1,2-dimyristoyl-rac-glycerol-methoxy(poly(ethylene glycol)) is about 50/10/38.5/1.5.

In some embodiments, the nanoparticle comprises C12-200, 1,2-dioleoyl-snglycero-3-phosphoethanolamine (DOPE), cholesterol, and 1,2-dimyristoyl-rac-glycerol-methoxy(poly(ethylene glycol)). In some embodiments, the molar ratio of C12-200, 1,2-dioleoyl-snglycero-3-phosphoethanolamine (DOPE), cholesterol, and 1,2-dimyristoyl-rac-glycerol-methoxy(poly(ethylene glycol)) is about 35/16/46.5/2.5.

In some embodiments, the nanoparticle disclosed herein comprises 5A2-SC8, 1,2-dioleoyl-snglycero-3-phosphoethanolamine (DOPE), cholesterol, 1,2-dimyristoyl-rac-glycerol-methoxy(poly(ethylene glycol)), and 1,2-dioleoyl-3-trimethylammonium-propane (DOTAP). The nanoparticle may comprise about 0.1% to about 30% mol/mol of DOTAP. For example, the amount of DOTAP present in the nanoparticle can be about 0.1%, about 0.2%, about 0.3%, about 0.4%, about 0.5%, about 0.6% mol/mol, about 0.7% mol/mol, about 0.8% mol/mol, about 0.9% mol/mol, about 1% mol/mol, about 2% mol/mol, about 2.5% mol/mol, about 3% mol/mol, about 3.5% mol/mol, about 4% mol/mol, about 4.5% mol/mol, about 5% mol/mol, about 5.5% mol/mol, about 6% mol/mol, about 6.5% mol/mol, about 7% mol/mol, about 7.5% mol/mol, about 8% mol/mol, about 8.5% mol/mol, about 9% mol/mol, about 9.5% mol/mol, about 10% mol/mol, about 10.5% mol/mol, about 11% mol/mol, about 11.5% mol/mol, about 12% mol/mol, about 12.5% mol/mol, about 13% mol/mol, about 13.5% mol/mol, about 14% mol/mol, about 15% mol/mol, about 16% mol/mol, about 17% mol/mol, about 18% mol/mol, about 19% mol/mol, about 20% mol/mol, about 22% mol/mol, about 24% mol/mol, about 26% mol/mol, about 28% mol/mol, about 30% mol/mol of its nanoparticle. In some embodiments, the amount of DOTAP present in the nanoparticle is about 20% mol/mol of its nanoparticle.

In some embodiments, the nanoparticles and methods for liver-specific delivery disclosed herein are the ones described in the art, e.g., in Cheng et al., Nat Nanotechnol. 2020 Apr.; 15(4):313-320. Epub 2020 Apr. 6; Trepotec et al., Mol Ther. 2019 Apr. 10; 27(4):794-802. Epub 2018 Dec. 22; Truong, et al., Proc Natl Acad Sci USA. 2019 Oct. 15; 116(42):21150-21159. Epub 2019 Sep. 9; which are incorporated herein by reference in their entireties.

In further embodiments, the vector disclosed herein comprises poly(amido-amine), poly-beta amino-esters (PBAEs), and/or polyethylenimine (PEI). In some embodiments, the vector comprises polyacridine PEG. In some embodiments, the vector disclosed herein comprises an outer PEG shell and a nanoparticle-based core.

Lipid-based nanoparticles successfully deliver therapeutic payloads to the liver. See, e.g., Witzigmann et al., Adv Drug Deliv Rev. 2020 Jul., doi: 10.1016/j.addr.2020.06.026. Liposomes can be made from several different types of lipids; however, phospholipids are most commonly used to generate lipid-based nanoparticles as drug carriers. Lipid particles for use in this invention may be prepared to include liposome-forming lipids and phospholipids, and membrane active sterols (e.g. cholesterol). Liposomes may include other lipids and phospholipids which are not liposome forming lipids.

Phospholipids may be selected, for example, from a lecithin (such as egg or soybean lecithin); a phosphatidylcholine (such as egg phosphatidylcholine); a hydrogenated phosphotidylcholine; a lysophosphatidyl choline; dipalmitoylphosphatidylcholine; distearoyl phosphatidylcholine; dimyristoyl phosphatidylcholine; dilauroylphosphatidylcholine; a glycerophospholipid (such as phosphatidylglycerol, phosphatidylserine, phosphatidylethanolamine, lysophosphatidylethanolamine, phosphatidylinositol, phosphatidylinositol phosphate, phosphatidylinositol bisphosphate and phosphatidylinositol triphosphate); sphingomyelin; cardiolipin; a phosphatidic acid; a plasmalogen; or a mixture thereof. Each possibility represents a separate embodiment of the invention. Examples of other lipids that can be used include a glycolipid (such as a glyceroglycolipid, e.g. a galactolipid and a sulfolipid, a glycosphingolipid, e.g., a cerebroside, a glucocerebroside and a galactocerebroside, and a glycosylphosphatidylinositol); a phosphosphingolipid (such as a ceramide phosphorylcholine, a ceramide phosphorylethanolamine and a ceramide phosphorylglycerol); or a mixture thereof. Each possibility represents a separate embodiment of the invention. Negatively or positively charged lipid nanoparticles can be obtained, for example, by using anionic or cationic phospholipids or lipids. Such anionic/cationic phospholipids or lipids typically have a lipophilic moiety, such as a sterol, an acyl or diacyl chain, and where the lipid has an overall net negative/positive charge.

In some embodiments, the nanoparticles disclosed herein include one, two, three or more biocompatible and/or biodegradable polymers. For example, a contemplated nanoparticle may include about 10 to about 99 weight percent of a one or more block co-polymers that include a biodegradable polymer and polyethylene glycol, and about 0 to about 50 weight percent of a biodegradable homopolymer. Polymers can include, for example, both biostable and biodegradable polymers, such as microcrystalline cellulose, hydroxypropyl cellulose, hydroxypropyl methylcellulose, polyalkylene oxides such as polyethylene oxide (PEG), polyanhydrides, poly(ester anhydrides), polyhydroxy acids such as polylactide (PLA), polyglycolide (PGA), poly(lactide-co-glycolide) (PLGA), poly-3-hydroxybutyrate (PHB) and copolymers thereof, poly-4-hydroxybutyrate (P4HB) and copolymers thereof, polycaprolactone and copolymers thereof, and combinations thereof.

In some embodiments, the nanoparticle has a diameter from about 1 nm to about 1000 nm. In some embodiments, the nanoparticle has a diameter less than, for example, about 1000 nm, about 950 nm, about 900 nm, about 850 nm, about 800 nm, about 750 nm, about 700 nm, about 650 nm, about 600 nm, about 550 nm, about 500 nm, about 450 nm, about 400 nm, about 350 nm, about 300 nm, about 290 nm, about 280 nm, about 270 nm, about 260 nm , about 250 nm, about 240 nm, about 230 nm, about 220 nm, about 210 nm, about 200 nm, about 190 nm, about 180 nm, about 170 nm, about 160 nm, about 150 nm, about 140 nm, about 130 nm, about 120 nm, about 110 nm, about 100 nm, about 90 nm, about 80 nm, about 70 nm, about 60 nm, about 50 nm, about 40 nm, about 30 nm, about 20 nm, or about 10 nm. In some embodiments, the nanoparticle has a diameter, for example, from about 20 nm to about 1000 nm, from about 20 nm to about 800 nm, from about 20 nm to about 700 nm, from about 30 nm to about 600 nm, from about 30 nm to about 500 nm, from about 40 nm to about 400 nm, from about 40 nm to about 300 nm, from about 40 nm to about 250 nm, from about 50 nm to about 250 nm, from about 50 nm to about 200 nm, from about 50 nm to about 150 nm, from about 60 nm to about 150 nm, from about 70 nm to about 150 nm, from about 80 nm to about 150 nm, from about 90 nm to about 150 nm, from about 100 nm to about 150 nm, from about 110 nm to about 150 nm, from about 120 nm to about 150 nm, from about 90 nm to about 140 nm, from about 90 nm to about 130 nm, from about 90 nm to about 120 nm, from 100 nm to about 140 nm, from about 100 nm to about 130 nm, from about 100 nm to about 120 nm, from about 100 nm to about 110 nm, from about 110 nm to about 120 nm, from about 110 nm to about 130 nm, from about 110 nm to about 140 nm, from about 90 nm to about 200 nm, from about 100 nm to about 195 nm, from about 110 nm to about 190 nm, from about 120 nm to about 185 nm, from about 130 nm to about 180 nm, from about 140 nm to about 175 nm, from 150 nm to 175nm, or from about 150 nm to about 170 nm. In some embodiments, the nanoparticle has a diameter from about 100 nm to about 250 nm. In some embodiments, the nanoparticle has a diameter from about 150 nm to about 175 nm. In some embodiments, the nanoparticle has a diameter from about 135 nm to about 175 nm. The particles can have any shape but are generally spherical in shape.

In some embodiments, the vector used herein is an exosome. The terms “microvesicle” and “exosomes,” as used herein, refer to a membranous particle having a diameter (or largest dimension where the particles is not spheroid) of between about 10 nm to about 5000 nm, more typically between 30 nm and 1000 nm, and most typically between about 50 nm and 750 nm, wherein at least part of the membrane of the exosomes is directly obtained from a cell. Most commonly, exosomes will have a size (average diameter) that is up to 5% of the size of the donor cell. Therefore, especially contemplated exosomes include those that are shed from a cell. Methods for making exosomes are known in the art. See, e.g., U.S. Publication No. 2018/0177727, incorporated by reference herein in its entirety. The exosomes and uses thereof for delivering polynucleotides and polypeptides are known in the art. See U.S. Pat. No. 10,577,630, incorporated by reference herein in its entirety.

Also included herein are compositions that increase expression or function of one or more transcription factors selected from the group consisting of PROX1, NR5A2, NR0B2, MTF1, SREBP1, EP300, and POM121C via RNA activation (RNAa). Accordingly, in some embodiments, the composition comprises short hairpin RNA (shRNA) that activates one or more of PROX1, NR5A2, NR0B2, MTF1, SREBP1, EP300, and POM121C.

“HNF4α” refers herein to a polypeptide that, in humans, is encoded by the HNF4A gene. In some embodiments, the HNF4α polypeptide is that identified in one or more publicly available databases as follows: HGNC: 5024, Entrez Gene: 3172, Ensembl ENSG00000101076, OMIM: 600281, UniProtKB: P41235. In some embodiments, the HNF4α polypeptide comprises the sequence of SEQ ID NO: 1 (HNF4α isoform 2), or a polypeptide sequence having at or greater than about 80%, about 85%, about 90%, about 95%, or about 98% homology with SEQ ID NO: 1, or a polypeptide comprising a portion of SEQ ID NO: 1. The HNF4α polypeptide of SEQ ID NO:1 may represent an immature or pre-processed form of mature HNF4α, and accordingly, included herein are mature or processed portions of the HNF4α polypeptide in SEQ ID NO: 1. In some embodiments, the HNF4α polypeptide is that described in U.S. Patent Application Publication US 2014/0249209, which is incorporated herein by reference for all purposes. In some embodiments, the HNF4α polynucleotide comprises the sequence of SEQ ID NO: 31, SEQ ID NO: 32, SEQ ID NO: 33, or SEQ ID NO: 34, or a polynucleotide sequence having at or greater than about 80%, about 85%, about 90%, about 95%, or about 98% homology with SEQ ID NO: 31, SEQ ID NO: 32, SEQ ID NO: 33, or SEQ ID NO: 34, or a polynucleotide comprising a portion of SEQ ID NO: 31, SEQ ID NO: 32, SEQ ID NO: 33, or SEQ ID NO: 34.

PROX1 (Prospero-related homeobox 1) is usually first expressed at embryonic day 8.5 (E8.5) in the endodermal cells of mice during liver organogenesis. In adult liver, PROX1's role can be controlling the energy metabolism of hepatocytes. More importantly, it has been reported that Prox1 may function as an activator of gene transcription by direct binding of its homeodomain to specific DNA elements. “PROX1” refers herein to a polypeptide that, in humans, is encoded by the PROX1 gene. In some embodiments, the PROX1 polypeptide is that identified in one or more publicly available databases as follows: HGNC: 9459, Entrez Gene: 5629, Ensembl: ENSG00000117707, OMIM: 601546, UniProtKB: Q92786. In some embodiments, the PROX1 polypeptide comprises the sequence of SEQ ID NO: 2, or a polypeptide sequence having at or greater than about 80%, about 85%, about 90%, about 95%, or about 98% homology with SEQ ID NO: 2, or a polypeptide comprising a portion of SEQ ID NO: 2. The PROX1 polypeptide of SEQ ID NO: 2 may represent an immature or pre-processed form of mature PROX1, and accordingly, included herein are mature or processed portions of the PROX1 polypeptide in SEQ ID NO: 2. In some embodiments, the PROX1 polynucleotide comprises the sequence of SEQ ID NO: 13, or a polynucleotide sequence having at or greater than about 80%, about 85%, about 90%, about 95%, or about 98% homology with SEQ ID NO: 13, or a polynucleotide comprising a portion of SEQ ID NO: 13.

NR5A2 (Nuclear receptor 5A2; Liver receptor homologue-1; LRH-1) is a nuclear receptor that binds as a monomer to a specific response element within the promoter and regulatory regions of its target genes. NR5A2 can also positively regulate genes encoding bile acid production enzymes, fatty acid metabolism and mitochondria function. “NR5A2” refers herein to a polypeptide that, in humans, is encoded by the NR5A2 gene. In some embodiments, the NR5A2 polypeptide is that identified in one or more publicly available databases as follows: HGNC: 7984, Entrez Gene: 2494, Ensembl: ENSG00000116833, OMIM: 604453, UniProtKB: O00482. In some embodiments, the NR5A2 polypeptide comprises the sequence of SEQ ID NO: 3, or a polypeptide sequence having at or greater than about 80%, about 85%, about 90%, about 95%, or about 98% homology with SEQ ID NO: 3, or a polypeptide comprising a portion of SEQ ID NO: 3. The NR5A2 polypeptide of SEQ ID NO: 3 may represent an immature or pre-processed form of mature NR5A2, and accordingly, included herein are mature or processed portions of the NR5A2 polypeptide in SEQ ID NO: 3. In some embodiments, the NR5A2 polynucleotide comprises the sequence of SEQ ID NO: 14, SEQ ID NO: 15, or SEQ ID NO: 16, or a polynucleotide sequence having at or greater than about 80%, about 85%, about 90%, about 95%, or about 98% homology with SEQ ID NO: 14, SEQ ID NO: 15, or SEQ ID NO: 16, or a polynucleotide comprising a portion of SEQ ID NO: 14, SEQ ID NO: 15, or SEQ ID NO: 16.

NR0B2 (Nuclear receptor small heterodimer partner; SHP) is usually highly expressed in normal hepatocytes and acts as an important transcriptional regulator for bile acid, glucose, and lipid metabolism. SUMOylation of NR0B2 can be required for nuclear transport and the gene repression function of SHP in feedback inhibition of Bile Acids biosynthesis that is critical for maintaining Bile Acids homoeostasis and protecting against liver toxicity. (Kim DH et al., 2016). “NR0B2” refers herein to a polypeptide that, in humans, is encoded by the NR0B2 gene. In some embodiments, the NR0B2 polypeptide is that identified in one or more publicly available databases as follows: HGNC: 7961, Entrez Gene: 8431, Ensembl ENSG00000131910, OMIM: 604630, UniProtKB: Q15466. In some embodiments, the NR0B2 polypeptide comprises the sequence of SEQ ID NO: 4, or a polypeptide sequence having at or greater than about 80%, about 85%, about 90%, about 95%, or about 98% homology with SEQ ID NO: 4, or a polypeptide comprising a portion of SEQ ID NO: 4. The NR0B2 polypeptide of SEQ ID NO: 4 may represent an immature or pre-processed form of mature NR0B2, and accordingly, included herein are mature or processed portions of the NR0B2 polypeptide in SEQ ID NO: 4. In some embodiments, the NR0B2 polynucleotide comprises the sequence of SEQ ID NO: 17, or a polynucleotide sequence having at or greater than about 80%, about 85%, about 90%, about 95%, or about 98% homology with SEQ ID NO: 17, or a polynucleotide comprising a portion of SEQ ID NO: 17.

MTF1 (Metal-responsive transcription factor 1) can mediate both basal and heavy metal-induced transcription of metallothionein genes and also can regulate other genes involved in the cell stress response and in metal homeostasis. MTF1 can also be involved in the transcriptional regulation of other metal-responsive genes, such as zinc transporter 1. MTF1 can regulates levels of zinc in hepatocytes“MTF1” refers herein to a polypeptide that is a self-ligand receptor of the signaling lymphocytic activation molecule family, and in humans, is encoded by the MTF1 gene. In some embodiments, the MTF1 polypeptide is that identified in one or more publicly available databases as follows: HGNC: 7428, Entrez Gene: 4520, Ensembl: ENSG00000188786, OMIM: 600172, UniProtKB: Q14872. In some embodiments, the MTF1 polypeptide comprises the sequence of SEQ ID NO: 5, or a polypeptide sequence having at or greater than about 80%, about 85%, about 90%, about 95%, or about 98% homology with SEQ ID NO: 5, or a polypeptide comprising a portion of SEQ ID NO: 5. The MTF1 polypeptide of SEQ ID NO: 5 may represent an immature or pre-processed form of mature MTF1, and accordingly, included herein are mature or processed portions of the MTF1 polypeptide in SEQ ID NO: 5. In some embodiments, the MTF1 polynucleotide comprises the sequence of SEQ ID NO: 18, or a polynucleotide sequence having at or greater than about 80%, about 85%, about 90%, about 95%, or about 98% homology with SEQ ID NO: 18, or a polynucleotide comprising a portion of SEQ ID NO: 18.

SREBP1 (Sterol regulator element binding proteins 1) is a transcription factor involved in the biogenesis of cholesterol, fatty acids and triglycerides. SREBP1 can control the expression and activity of the AKT/PI3K signaling pathway and vice-versa. (Shi Q et al., 2016; Porstmann T et al., 2008). “SREBF1” refers herein to a polypeptide that is a self-ligand receptor of the signaling lymphocytic activation molecule family, and in humans, is encoded by the SREBF1 gene. In some embodiments, the SREBF1 polypeptide is that identified in one or more publicly available databases as follows: HGNC: 11289, Entrez Gene: 6720, Ensembl: ENSG00000072310, OMIM: 184756, UniProtKB: P36956. In some embodiments, the SREBF1 polypeptide comprises the sequence of SEQ ID NO: 6, or a polypeptide sequence having at or greater than about 80%, about 85%, about 90%, about 95%, or about 98% homology with SEQ ID NO: 6, or a polypeptide comprising a portion of SEQ ID NO: 6. The MTF1 polypeptide of SEQ ID NO: 6 may represent an immature or pre-processed form of mature SREBF1, and accordingly, included herein are mature or processed portions of the SREBF1 polypeptide in SEQ ID NO: 6. In some embodiments, the SREBP1 polynucleotide comprises the sequence of SEQ ID NO: 19, SEQ ID NO: 20, or SEQ ID NO: 21, or a polynucleotide sequence having at or greater than about 80%, about 85%, about 90%, about 95%, or about 98% homology with SEQ ID NO: 19, SEQ ID NO: 20, or SEQ ID NO: 21, or a polynucleotide comprising a portion of SEQ ID NO: 19, SEQ ID NO: 20, or SEQ ID NO: 21.

EP300 (The histone acetyltransferase p300) EP300 can form complexes with C/EBP proteins and activate promoters of genes involved in triglyceride synthesis during the development of hepatic steatosis, glucose metabolism, and the regulation of several transcription factors, such as Foxo1 and farnesoid X receptor (FXR), which are highly expressed in the liver. “EP300” refers herein to a polypeptide that is a self-ligand receptor of the signaling lymphocytic activation molecule family, and in humans, is encoded by the EP300 gene. In some embodiments, the EP300 polypeptide is that identified in one or more publicly available databases as follows: HGNC: 3373, Entrez Gene: 2033, Ensembl: ENSG00000100393, OMIM: 602700, UniProtKB: Q09472. In some embodiments, the EP300 polypeptide comprises the sequence of SEQ ID NO: 7, or a polypeptide sequence having at or greater than about 80%, about 85%, about 90%, about 95%, or about 98% homology with SEQ ID NO: 7, or a polypeptide comprising a portion of SEQ ID NO: 7. The EP300 polypeptide of SEQ ID NO: 7 may represent an immature or pre-processed form of mature EP300, and accordingly, included herein are mature or processed portions of the EP300 polypeptide in SEQ ID NO: 7. In some embodiments, the EP300 polynucleotide comprises the sequence of SEQ ID NO: 22 or SEQ ID NO: 23, or a polynucleotide sequence having at or greater than about 80%, about 85%, about 90%, about 95%, or about 98% homology with SEQ ID NO: 22 or SEQ ID NO: 23, or a polynucleotide comprising a portion of SEQ ID NO: 22 or SEQ ID NO: 23.

POM121C (Nuclear envelope pore membrane protein POM 121) is a membrane protein that is a member of a group of proteins referred to as pore membrane proteins that are believed to participate in nuclear pore biogenesis. “POM121C” refers herein to a polypeptide that is a self-ligand receptor of the signaling lymphocytic activation molecule family, and in humans, is encoded by the POM121C gene. In some embodiments, the POM121C polypeptide is that identified in one or more publicly available databases as follows: HGNC: 34005, Entrez Gene: 100101267, Ensembl ENSG00000272391, OMIM: 615754, UniProtKB: A8CG34. In some embodiments, the POM121C polypeptide comprises the sequence of SEQ ID NO:8, or a polypeptide sequence having at or greater than about 80%, about 85%, about 90%, about 95%, or about 98% homology with SEQ ID NO: 8, or a polypeptide comprising a portion of SEQ ID NO: 8. The POM121C polypeptide of SEQ ID NO: 8 may represent an immature or pre-processed form of mature POM121C, and accordingly, included herein are mature or processed portions of the POM121C polypeptide in SEQ ID NO: 8. In some embodiments, the POM121C polynucleotide comprises the sequence of SEQ ID NO: 24, or a polynucleotide sequence having at or greater than about 80%, about 85%, about 90%, about 95%, or about 98% homology with SEQ ID NO: 24, or a polynucleotide comprising a portion of SEQ ID NO: 24.

Also disclosed herein is a composition that decreases an amount or suppresses a function of one or more transcription factors selected from the group consisting of DNAJB1/HSP40, ATF6, ATF4, and PERK. Accordingly, included herein are compositions that comprise small activating RNA (saRNA) such as small interfering RNA (siRNA) and microRNA (miRNA), or CRISPR RNA such as crisgRNA or tracr/mate RNAs, that correlate with and/or act on DNAJB1/HSP40, ATF6, ATF4, and PERK polynucleotides. The methods of using saRNA to decrease or suppresses a function of a protein is known in the art. See, e.g., International Publication. No: WO2019/048632, incorporated by reference herein in its entirety. Accordingly, included herein are methods of increasing expression of HNF4α using saRNA to decreases an amount or suppresses a function of one or more transcription factors selected from the group consisting of DNAJB1/HSP40, ATF6, ATF4, and PERK. In some embodiments, the composition comprises a nucleic acid that decreases an amount or suppresses function of DNAJB1/HSP40. In some embodiments, the composition comprises a nucleic acid that decreases an amount or suppresses function of ATF6. In some embodiments, the composition comprises a nucleic acid that decreases an amount or suppresses function of ATF4. In some embodiments, the composition comprises a nucleic acid that decreases an amount or suppresses function of PERK. In some embodiments, the composition further comprises a nucleic acid that encodes HNF4α.

DNAJB1/HSP40 (Heat shock protein 40) is a molecular chaperone protein that can play an essential role in gene expression and translational initiation, folding and unfolding as well as translocation and degradation of proteins. The activity of DNAJs/HSP40s is regulated by several post-translational modifications. In many cases, DNAJs/HSP40s are phosphoproteins (e.g. DnaJA1, DnaJB4, DnaJC1, DnaJC29) whose expressions and functions can be further modulated co- and post-translationally by acetylation (e.g. DnaJA1, DnaJB2, DnaJB12, DnaJC5, DnaJC8, DnaJC13), glycosylation (DnaJB11, DnaJC10, DnaJC16), palmitoylation (DnaJC5, DnaJC5B, DnaJC5G), methylation (DnaJA1-4), prenylation (DnaJA1, DnaJA2, DnaJA4), and formation of intramolecular disulfide bonds (DnaJB11, DnaJC3, DnaJC10), respectively. “DNAJB1/HSP40” refers herein to a polypeptide that is a self-ligand receptor of the signaling lymphocytic activation molecule family, and in humans, is encoded by the DNAJB1 gene. In some embodiments, the DNAJB1 polypeptide is that identified in one or more publicly available databases as follows: HGNC: 5270, Entrez Gene: 3337, Ensembl: ENSG00000132002, OMIM: 604572, UniProtKB: P25685. In some embodiments, the DNAJB1 polypeptide comprises the sequence of SEQ ID NO: 9, or a polypeptide sequence having at or greater than about 80%, about 85%, about 90%, about 95%, or about 98% homology with SEQ ID NO: 9, or a polypeptide comprising a portion of SEQ ID NO: 9. The DNAJB1 polypeptide of SEQ ID NO: 9 may represent an immature or pre-processed form of mature DNAJB1, and accordingly, included herein are mature or processed portions of the DNAJB1 polypeptide in SEQ ID NO: 9. In some embodiments, the DNAJB1 polynucleotide comprises the sequence of SEQ ID NO: 25 or SEQ ID NO: 26, or a polynucleotide sequence having at or greater than about 80%, about 85%, about 90%, about 95%, or about 98% homology with SEQ ID NO: 25 or SEQ ID NO: 26, or a polynucleotide comprising a portion of SEQ ID NO: 25 or SEQ ID NO: 26.

ATF6 can be a sensor of the unfolded protein response (UPR) and function to regulate transcriptional expression. It has been shown that in some instances, upon ER stress, ATF6 is trafficked from the ER to the Golgi, where it is proteolytically cleaved, releasing the N-terminal ATF6 segment, a transcription factor of genes involved in the folding and trafficking of proteins. “ATF6” refers herein to a polypeptide that is a self-ligand receptor of the signaling lymphocytic activation molecule family, and in humans, is encoded by the ATF6 gene. In some embodiments, the ATF6 polypeptide is that identified in one or more publicly available databases as follows: HGNC: 791, Entrez Gene: 22926, Ensembl ENSG00000118217, OMIM: 605537, UniProtKB: P18850. In some embodiments, the ATF6 polypeptide comprises the sequence of SEQ ID NO: 10, or a polypeptide sequence having at or greater than about 80%, about 85%, about 90%, about 95%, or about 98% homology with SEQ ID NO: 10, or a polypeptide comprising a portion of SEQ ID NO: 10. The ATF6 polypeptide of SEQ ID NO: 10 may represent an immature or pre-processed form of mature ATF6, and accordingly, included herein are mature or processed portions of the ATF6 polypeptide in SEQ ID NO: 10. In some embodiments, the ATF6 polynucleotide comprises the sequence of SEQ ID NO: 27, or a polynucleotide sequence having at or greater than about 80%, about 85%, about 90%, about 95%, or about 98% homology with SEQ ID NO: 27, or a polynucleotide comprising a portion of SEQ ID NO: 27.

ATF4 a transcriptional activator of UPR target genes that can serve to enhance transcriptional expression of genes involved in amino acid metabolism and resistance to oxidative stress. (Fusakio ME et al., 2016) “ATF4” refers herein to a polypeptide that is a self-ligand receptor of the signaling lymphocytic activation molecule family, and in humans, is encoded by the ATF4 gene. In some embodiments, the ATF4 polypeptide is that identified in one or more publicly available databases as follows: HGNC: 786, Entrez Gene: 468, Ensembl: ENSG00000128272, OMIM: 604064, UniProtKB: P18848. In some embodiments, the ATF4 polypeptide comprises the sequence of SEQ ID NO: 11, or a polypeptide sequence having at or greater than about 80%, about 85%, about 90%, about 95%, or about 98% homology with SEQ ID NO: 11, or a polypeptide comprising a portion of SEQ ID NO: 11. The ATF4 polypeptide of SEQ ID NO: 11 may represent an immature or pre-processed form of mature ATF4, and accordingly, included herein are mature or processed portions of the ATF4 polypeptide in SEQ ID NO: 11. In some embodiments, the ATF4 polynucleotide comprises the sequence of SEQ ID NO: 28, or a polynucleotide sequence having at or greater than about 80%, about 85%, about 90%, about 95%, or about 98% homology with SEQ ID NO: 28, or a polynucleotide comprising a portion of SEQ ID NO: 28.

PERK (protein kinase RNA-like endoplasmic reticulum kinase) is a type 1 transmembrane protein that is typically activated through recruitment of chaperones away from PERK leading to oligomerization and activation of the cytosolic kinase domain. A critical protein that mediates proapoptotic signaling downstream of PERK and ATF4 is CCAAT enhancer-binding protein (C/EBP) homologous protein (CHOP), which is implicated in the progression of liver disease. (Malhi H et al., 2011) “PERK”, also known as “EIF2AK3” refers herein to a polypeptide that is a self-ligand receptor of the signaling lymphocytic activation molecule family, and in humans, is encoded by the EIF2AK3 gene. In some embodiments, the PERK polypeptide is that identified in one or more publicly available databases as follows: NC: 3255, Entrez Gene: 9451, Ensembl ENSG00000172071, OMIM: 604032, UniProtKB: Q9NZJ5. In some embodiments, the PERK polypeptide comprises the sequence of SEQ ID NO: 12, or a polypeptide sequence having at or greater than about 80%, about 85%, about 90%, about 95%, or about 98% homology with SEQ ID NO: 12, or a polypeptide comprising a portion of SEQ ID NO: 12. The PERK polypeptide of SEQ ID NO: 12 may represent an immature or pre-processed form of mature PERK, and accordingly, included herein are mature or processed portions of the PERK polypeptide in SEQ ID NO: 12. In some embodiments, the PERK or EIF2AK3 polynucleotide comprises the sequence of SEQ ID NO: 29 or SEQ ID NO: 30, or a polynucleotide sequence having at or greater than about 80%, about 85%, about 90%, about 95%, or about 98% homology with SEQ ID NO: 29 or SEQ ID NO: 30, or a polynucleotide comprising a portion of SEQ ID NO: 29 or SEQ ID NO: 30.

The composition of any preceding aspects may further comprise a HNF4α agonist, wherein the HNF4α agonist is meant to refer to those compositions described more fully in U.S. Patent Application Publication US2014/0249209, incorporated herein by reference for all purposes.

In some embodiments, the composition and/or the vector of any preceding aspects can be formulated with a biologically acceptable carrier. In some embodiments, the biologically acceptable carrier is capable of transferring the composition and/or the vector into and/or between host cells. In some embodiments, the biologically acceptable carrier is capable of transferring the composition and/or the vector into and/or between target cells, such as a hepatocyte in the liver of a subject. Importantly, in some embodiments, the composition and/or the vector along with the biologically acceptable carrier is capable of transporting functional macromolecules such as DNA and RNA into a nucleus of a target cell (e.g., a hepatocyte).

### Methods of Treatment

Provided herein are methods of treating a liver disease by increasing the expression and/or transport and/or retention of HNF4α, a transcriptional factor, into a nucleus of a hepatocyte in the subject. In some embodiments, the method comprises upregulating expression or function of one or more transcription factors selected from the group consisting of PROX1, NR5A2, NR0B2, MTF1, SREBP1, EP300, and POM121C and/or downregulating expression or function of one or more transcription factors selected from the group consisting of DNAJB1/HSP40, ATF6, ATF4, and PERK. In some aspects, disclosed herein is a method of treating a liver disease in a subject in need thereof comprising administering to the subject a vector, wherein the vector comprises a nucleic acid that encodes one or more transcription factors selected from the group consisting of PROX1, NR5A2, NR0B2, MTF1, SREBP1, EP300, and/or POM121C, and functional fragments thereof. In some aspects, disclosed herein is a method of treating a liver disease in a subject in need thereof comprising administering to the subject a composition that downregulates expression or function of one or more transcription factors selected from the group consisting of DNAJB1/HSP40, ATF6, ATF4, and PERK. In some aspects, the composition comprises a siRNA, miRNA, sgRNA or tracr/mate RNA. In other embodiments, the method comprises increasing acetylation of HNF4α at Lys106, increasing expression of cMET, and/or increasing activation of AKT via phosphorylation at Thr308.

In some embodiments, disclosed herein is a method of treating a liver disease in a subject in need thereof comprising administering to the subject a vector, wherein the vector comprises a nucleic acid that encodes PROX1. In some embodiments, the method of treating a liver disease in a subject in need thereof that comprises administering to the subject a vector, wherein the vector comprises a nucleic acid that encodes SREBP1. In some embodiments, the vector further comprises a nucleic acid that encodes HNF4α. In some embodiments, the vector comprises one or more nucleic acids that encode HNF4α, PROX1, and SREBP1. In some embodiments, the method further comprises administering a vector that comprises a nucleic acid that encodes HNF4α.

As noted above, PROX1, NR5A2, NR0B2, MTF1, SREBP1, EP300, and POM121C are all transcription factors and/or regulators that regulate HNF4α nuclear transportation through direct or indirect mechanisms, including, for example, acetylation of HNF4α, cell metabolism pathways, or the formation of nuclear pore complex. Such effects on HNF4α can restore liver cell function in patients having liver diseases.

Accordingly, in some embodiments, the administration of the vector or vectors increases an amount of HNF4α in a nucleus of a hepatocyte in the subject. In some embodiments, the administration of the vector(s) does not increase a total amount of HNF4α in the hepatocyte. In some embodiments, the administration of the vector(s) increases a total amount of HNF4α in the hepatocyte. In some embodiments, the vector of any preceding aspects further comprises a nucleic acid that encodes HNF4α. In some embodiments, the vector disclosed herein further comprises a nucleic acid encoding an HNF4α isoform 2 polypeptide. In some embodiments, the HNF4α isoform 2 polypeptide comprises a sequence at least about 80%, about 85%, about 90%, about 95%, or about 98% identical to SEQ ID NO: 1 or a fragment thereof. In some embodiments, the nucleic acid at least about 80%, about 85%, about 90%, about 95%, or about 98% identical to SEQ ID NO: 31 or a fragment thereof.

Accordingly, in some aspects, disclosed herein is a method of treating a liver disease in a subject in need thereof comprising administering to the subject a vector that comprises a nucleic acid encoding HNF4α isoform 2. Also included herein is a use of a composition for the preparation of a medicament for treatment a liver disease in a subject in need thereof comprising administering to the subject the composition, wherein the composition comprises a nucleic acid encoding HNF4α isoform 2.

The vector used in the methods may be any as described herein including plasmid, bacteriophage, viral particle (isolated, attenuated, recombinant, etc.), exosome, extracellular vesicle and/or nanoparticle. In some embodiments, the vector is a plasmid. In some embodiments, the vector is a viral particle. In some embodiments, the vector is viral vector with a natural and/or an engineered capsid. In some embodiments, the vector is an exosome. In some embodiments the vector is a nanoparticle. In some embodiments, the vector is an mRNA lipid nanoparticle. In some embodiments, the nucleic acid is a DNA (e.g., closed-ended DNA (ceDNA)) or an RNA. ceDNAs and methods of making and using ceDNA are known in the art. For example, see International Publication Nos. W02019/169233 and WO2017152149, incorporated by reference herein in their entireties. With respect to general information on nanoparticles, components thereof and delivery of such components, including methods, materials, delivery nanoparticles and making and using thereof, including as to amounts and formulations, all useful in the practice of the instant invention, reference is made to Wu et al., J. Biol. Chem. 262, 4429, 1987, U.S. Patent Application Publication 2011/0274706, and WO2018/170405, which are incorporated herein by reference for all purposes.

In some aspects, disclosed herein is a method of treating a liver disease in a subject in need thereof comprising administering to the subject a composition, wherein the composition decreases an amount or suppresses function of one or more transcription factors selected from the group consisting of DNAJB1/HSP40, ATF6, ATF4, and PERK.

As noted above, DNAJB1/HSP40, ATF6, ATF4, and PERK are all transcriptional regulators of endoplasmic reticulum (ER) stress. The ER is a type of membranous organelle in eukaryotic cells that is important for the proper folding, modification, and transportation of proteins. ER stress, occurring when the capacity of an ER to fold proteins becomes saturated, may lead to responses such as cell death and/inflammation. These transcriptional regulators regulate HNF4α nuclear transportation through pathways related to ER stress. It is shown herein that decreases in an amount or suppression of function of one or more of these transcriptional regulators can restore liver cell function in patients having a liver disease. Accordingly, in some embodiments, administration of the composition increases an amount of HNF4α in a nucleus of a hepatocyte in the subject. In some embodiments, administration of the composition does not increase a total amount of HNF4α in the hepatocyte. In some embodiments, the administration of the composition increases a total amount of HNF4α in the hepatocyte. In some embodiments, the composition further comprises a nucleic acid that encodes HNF4α.

In some embodiments, the composition decreases the amount of one or more transcription factors selected from the group consisting of DNAJB1/HSP40, ATF6, ATF4, and/or PERK by knockdown of these genes. Knockdown of DNAJB1/HSP40, ATF6, ATF4, and/or PERK may be brought about by recognition of relevant mRNA, such as mRNA encoding DNAJB1/HSP40, ATF6, ATF4, and/or PERK or enzymes necessary for DNAJB1/HSP40, ATF6, ATF4, and/or PERK activity, by a complementary RNA molecule, and mediated by RNA interference. For example, molecules encoding interfering RNA (RNAi) may be introduced into a hepatocyte or hepatocyte precursor by a suitable vector, such as a lentiviral, retroviral vector, or a nanoparticle, through methods such as transfection or transduction.

In some embodiments, any one or plurality of CRISPR complex components for knocking out the one or more transcription factors selected from the group consisting of DNAJB1/HSP40, ATF6, ATF4, and/or PERK may be administered with or within the viral particles, virions, or viral vectors disclosed herein. In some embodiments, an sgRNA or tracr/mate RNAs may be packaged with one or more reprogramming factors. In some embodiments, sgRNA molecules encapsulated by the viral particles, virions, or viral vectors may be packaged with one or more reprogramming factors. With respect to general information on CRISPRCas Systems, components thereof and delivery of such components, including methods, materials, delivery vehicles, vectors, particles, AAV, and making and using thereof, including as to amounts and formulations, all useful in the practice of the instant invention, reference is made to U.S. Publication 2018/0057839, which is incorporated herein by reference for all purposes.

As noted above, “liver disease” as used herein refers generally to diseases, disorders, and conditions affecting the liver, and may have a wide range of severity encompassing, for example, simple accumulation of fat in the hepatocytes (steatosis), macrovescicular steatosis, periportal and lobular inflammation (steatohepatitis), cirrhosis, fibrosis, liver ischemia, liver cancer including hepatocellular carcinoma, a liver disease of an earlier disease stage, end-stage liver disease, and liver failure. Accordingly, each of steatosis, macrovescicular steatosis, steatohepatitis, cirrhosis, fibrosis, liver cancer, hepatocellular carcinoma, end-stage liver disease, chronic liver disease, and liver failure are included within the definition of “liver disease.” The degree or severity of “liver cirrhosis” can be designated by a Child-Pugh score wherein five clinical measures, levels of total bilirubin, serum albumin, prothrombin time prolongation, ascites, and hepatic encephalopathy, are scored using a point system of 1 point, 2 point, and 3 point values for varying levels of each clinical measure, with 3 point values being assigned to the most severe levels of each measure. The total points for all five measures are added to arrive at a Child-Pugh score and classification. Scores of 5-6 designate Child-Pugh Class A, scores of 7-9 designate Child-Pugh Class B, and scores of 10-15 designate Child-Pugh Class C. In general, Child-Pugh Class A indicates the least severe liver disease and Child-Pugh Class C indicates the most severe liver disease. In some embodiments, the method disclosed herein can be used to treat a subject having a Child-Pugh Class B or Child-Pugh Class B C liver disease. In some embodiments, the method disclosed here in can be used to treat a subject having a Child-Pugh Class A liver disease. In some embodiments, the liver disease is alcoholic hepatitis. In some embodiments, the method disclosed here in can be used to treat an ischemic donor liver having ex vivo perfusion. The present invention can be used to treat liver cancer before or after cancer treatment including before or after liver resection. It should be understood and herein contemplated that a liver disease of an earlier disease stage can be a nonalcoholic fatty liver disease (NAFLD), a nonalcoholic steatohepatitis (NASH), an alcohol-related liver disease, including but not limited to, fatty liver, alcoholic hepatitis, and alcohol-related cirrhosis. It should also be understood that the end-stage liver disease disclosed herein can be attributed to all causes known the art, including, for examples, viral, alcoholic, non-alcoholic, and cryptogenic.

Accordingly, the methods disclosed herein can be used to improve liver function in a subject having the liver disease of any preceding aspects. Such improvement of liver function can be indicated by, for example, an increase in serum albumin, a decrease in serum ammonia level, a decrease in total bilirubin, an increase in encephalopathy score, and/or a decrease in prothrombin time prolongation. Accordingly, the methods disclosed herein can be used to increase serum albumin levels, decrease serum ammonia levels, decrease total bilirubin levels, increase encephalopathy score, and/or decrease prothrombin time prolongation.

A liver disease may progress with multiple stages, including, inflammation, fibrosis, cirrhosis, end-stage liver disease, and liver cancer. It should be known that there are numerous causes of chronic liver disease, including chronic infection by hepatitis viruses, alcohol-mediated cirrhosis, and/or non-alcoholic steatohepatitis (NASH). As the timing of a liver disease can often not be predicted, it should be understood the disclosed methods of treating, preventing, reducing, and/or inhibiting a liver disease, can be used prior to or following the onset of inflammation, fibrosis, cirrhosis, end-stage liver disease, and/or liver cancer, even prior to or during hepatitis virus infection, alcohol-mediated cirrhosis, and/or non-alcoholic steatohepatitis, to treat, prevent, inhibit, and/or mitigate any stage of the liver disease. The disclosed methods can be performed any time prior to the onset of inflammation, fibrosis, cirrhosis, end-stage liver disease, and/or liver cancer. In one aspect, the disclosed methods can be employed 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 years; 12, 11, 10, 9, 8, 7, 6, 5, 4, 3, 2, or 1 months; 30, 29, 28, 27, 26, 25, 24, 23, 22, 21, 20, 19, 18, 17, 16, 15, 14, 13, 12, 11, 10, 9, 8, 7, 6, 5, 4, or 3 days; 60, 48, 36, 30, 24, 18, 15, 12, 10, 9, 8, 7, 6, 5, 4, 3, or 2 hours prior to the onset of inflammation, fibrosis, cirrhosis, end-stage liver disease, and/or liver cancer; or 1, 2, 3, 4, 5, 6, 7, 8, 9, 10, 15, 20, 25, 30, 35, 40, 45, 50, 55, 60, 75, 90, 105, 120 minutes; 3, 4, 5, 6, 7, 8, 9, 10, 11, 12, 15, 18, 24, 30, 36, 48, 60 hours; 3, 4, 5, 6, 7, 8, 9, 10, 11, 12, 13, 14, 15, 16, 17, 18, 19, 20, 21, 22, 23, 24, 25, 26, 27, 28, 29, 30, 45, 60, 90 or more days; 4, 5, 6, 7, 8, 9, 10, 11, 12 or more months; 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1 years after the onset of inflammation, fibrosis, cirrhosis, end-stage liver disease, and/or liver cancer.

Liver resection is the surgical removal of all or a portion of a liver of a subject having a liver disease (e.g., cirrhosis, end-stage liver disease, and/or liver cancer). The disclosed methods can be performed to the subject any time prior to or after liver resection. In one aspect, the disclosed methods can be employed 5, 4, 3, 2, or 1 years; 12, 11, 10, 9, 8, 7, 6, 5, 4, 3, 2, or 1 months; 30, 29, 28, 27, 26, 25, 24, 23, 22, 21, 20, 19, 18, 17, 16, 15, 14, 13, 12, 11, 10, 9, 8, 7, 6, 5, 4, or 3 days; 60, 48, 36, 30, 24, 18, 15, 12, 10, 9, 8, 7, 6, 5, 4, 3, or 2 hours prior to the operation of liver resection; or 1, 2, 3, 4, 5, 6, 7, 8, 9, 10, 15, 20, 25, 30, 35, 40, 45, 50, 55, 60, 75, 90, 105, 120 minutes; 3, 4, 5, 6, 7, 8, 9, 10, 11, 12, 15, 18, 24, 30, 36, 48, 60 hours; 3, 4, 5, 6, 7, 8, 9, 10, 11, 12, 13, 14, 15, 16, 17, 18, 19, 20, 21, 22, 23, 24, 25, 26, 27, 28, 29, 30, 45, 60, 90 or more days; 4, 5, 6, 7, 8, 9, 10, 11, 12 or more months; 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1 years after liver resection.

The vector or the composition described herein can be administered to the subject via any route including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques. In some embodiments, the administration of the vector or the composition is intravenous.

Another aspect of the disclosure relates to administering both the above-described composition that increases the amount or function of one or more transcription factors selected from the group consisting of PROX1, NR5A2, NR0B2, MTF1, SREBP1, EP300, and/or POM121C, and functional fragments thereof and the composition that decreases an amount or suppresses function of one or more transcription factors selected from the group consisting of DNAJB1/HSP40, ATF6, ATF4, and PERK. In some embodiments, both compositions are administered at the same time. In other embodiments, one composition is administered before the other. In some embodiments, the method of any preceding aspects further comprises upregulating expression or function of HNF4α. In some embodiments, the method of any preceding aspects comprises further administering a HNF4α agonist, which term refers to those compositions described more fully in U.S. Patent Application Publication US2014/0249209, which is incorporated herein by reference for all purposes.

Dosing frequency for the vector or the composition of any preceding aspects, includes, but is not limited to, at least once every year, once every two years, once every three years, once every four years, once every five years, once every six years, once every seven years, once every eight years, once every nine years, once every ten year, at least once every two months, once every three months, once every four months, once every five months, once every six months, once every seven months, once every eight months, once every nine months, once every ten months, once every eleven months, at least once every month, once every three weeks, once every two weeks, once a week, twice a week, three times a week, four times a week, five times a week, six times a week, or daily. Administration can also be continuous and adjusted to maintaining a level of the compound within any desired and specified range. The term “administration” or “administrating” used herein for treating a liver disease using the vector and/or the composition of any preceding aspect includes those forms of administration described more fully in U.S. Patent Application Publication 2018/0057839, which is incorporated herein by reference for all purposes.

### EXAMPLES

The following examples are set forth below to illustrate the compositions, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Methods and Materials

Human Samples and Hepatocyte Isolation. De-identified normal human liver tissue and/or cells were obtained through the Liver Tissue Cell Distribution System (Pittsburgh, Pa.) after obtaining written informed consent by a protocol approved by the Human Research Review Committee of the University of Pittsburgh, which was funded by NIH Contract #HSN276201200017C. Adult human liver tissue and/or cells were also obtained from Ira J Fox Laboratory at Children's Hospital of UPMC, by a protocol approved by the Human Research Review Committee and the Institutional Review Board (Exempt with identifiers removed; IRB #: PRO12090466) of the University of Pittsburgh (Table 1). Hepatocytes were isolated using a three-step collagenase digestion technique as previously described (Gramignoli R et al., 2012). Cell viability was assessed after isolation as previously described using trypan blue exclusion and only cell preparations with viability>80% were used for the analysis.

TABLE 1
CLINICAL PARAMETERS OF THE PATIENTS IN THIS STUDY

| | | | Total | | | | |
|---|---|---|---|---|---|---|---|
| | Age | Sex | Bilirubin | Albumin | | | |
| Etiology/Code, n | (years) | (M, F) | (mg/ [symbol] ) | ( [symbol] ) | [symbol] | Child-Pugh | |
| Healthy liver 4 | 64 ± 13 | 2.2 | — | — | — | — | |
| NASH 5 | 60 ± 10 | 2.3 | 1.75 ± 0.6 | 2.9 ± 0.1 | 12.4 ± 6.1 | 9.4 ± 0.9 | |
| NASH 1 | 71 | 0.1 | 1.7 | 3.0 | 8 | 9 | (8) |
| NASH 2 | 68 | 0.1 | 1.7 | 2.9 | 8 | 9 | (8) |
| NASH 3 | 53 | 1.0 | 1.1 | 2.8 | 8 | 9 | (8) |
| NASH 4 | 69 | 1.0 | 1.6 | 2.8 | 21 | 11 | ( [symbol] ) |
| NASH 5 | 48 | 0.1 | 2.7 | 3.0 | 17 | 9 | (8) |
| Alcohol 2 | 5 [symbol] ± 5 | 3.0 | 2.03 ± 1.0 | 2.53 ± 0.6 | 140 ± 2.6 | 10.0 ± 1.0 | |
| Alcohol 1 | 52 | 1.0 | 1. [symbol] | 1.9 | 16 | 11 | ( [symbol] ) |
| Alcohol 2 | 61 | 1.0 | 1.4 | 3.2 | 11 | 9 | (8) |

[symbol]

indicates data missing or illegible when filed

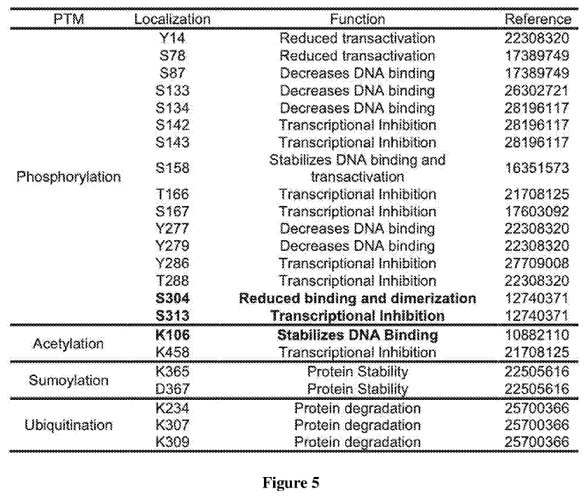

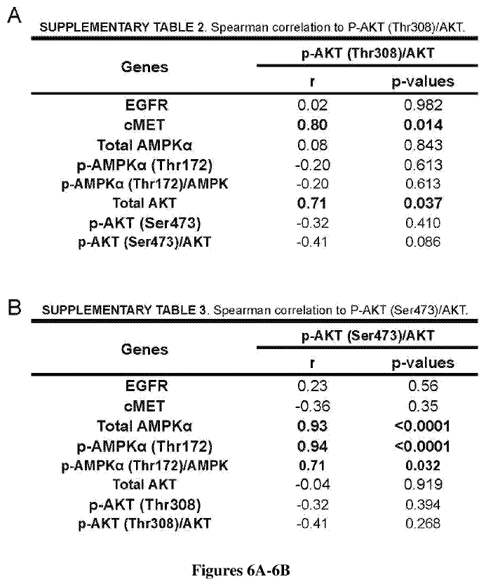

In silico HNF4α post-translational modifications (PTMs) analysis. To identify the PTMs that modulate HNF4α cellular localization, an in silico analysis was performed through computational searches in databases and publications (FIG. 5). The process was divided into three phases: identification, screening, and selection. Initially, 51 PTMs were identified. Next, during the screening phase, 23 PTMs were selected by the application of two elimination criteria (FIG. 5). Two phosphorylation and one acetylation modifications were identified in the selection phase as the most plausible PTMs related to HNF4α localization able to be evaluated.

Stable Isotope Analysis Using Gas Chromatography—Mass SpectrometryOne million human hepatocytes were cultured with Dulbecco's modified Eagle's medium F12 in the presence of 13-C6-labeled glucose and glutamine isotope tracers for 96 hours (Thermo Fisher Scientific, San Jose, Calif.). The medium was removed and cells were washed with ice-cold phosphate-buffered saline solution. Next, cells were quenched with 400 μL methanol and 400 μL water containing 1 μL norvaline, scraped, washed with 800 μL ice-cold chloroform, vortexed at 4° C. for 30 minutes, and centrifuged at 7,300 rpm for 10 minutes at 4° C. The upper aqueous phase was collected for metabolite analysis. Metabolite extracts were centrifuged at 14,000 g for 10 minutes to separate the polar phase, protein interphase, and chloroform phase. The water/methanol phase—containing polar metabolites were transferred to fresh microcentrifuge tubes and dried in a SpeedVac and stored at −80° C. until gas chromatography—mass spectrometry (GC-MS) analysis. Then, 30 μL of methoxyamine hydrochloride (Thermo Scientific) was added to dried samples and incubated at 30° C. for 2 hours with intermittent vortexing. A total of 45 μL of MBTSTFA+1% tert-butyldimethylchlorosilane was added to the samples and incubated at 55° C. for 1 hour. Derivatized samples were transferred to gas chromatography (GC) vials with glass inserts and added to the GC-MS autosampler. GC-MS analysis was performed using an Agilent 7890 GC (Santa Clara, Calif.) equipped with a 30-m HP-SMSUI capillary column connected to an Agilent 5977B mass spectrometer. For polar metabolites, the following heating cycle was used for the GC oven: 100° C. for 3 minutes, followed by a ramp of 5° C./minute to 300° C. and held at 300° C. for a total run time of 48 minutes. Data were acquired in scan mode. The relative abundance of metabolites was calculated from the integrated signal of all potentially labeled ions for each metabolite fragment. Mass isotopologue distributions were corrected for natural abundance using IsoCorrectoR prior to analysis with the model. Metabolite levels were normalized to internal standard Norvaline's signal. Fractional enrichment calculation represents the fractional contribution of 13C from a substrate to intermediate metabolite. It is calculated as follows:

$$MPE = ( {\sum }_{i = 0}^{{N}_{c}} i * {x}_{i} ) / {N}_{c}$$

where NC is the number of carbons that can be labeled as 13C, and xi is the fraction of (M+i)th isotopologue.

Immunohistochemistry and HNF4α quantification. Paraffin-embedded liver tissue was deparaffinized with xylenes and dehydrated with ethanol. Antigen unmasking was performed by boiling in citrate buffer, pH 6.0. The slides were then incubated in 3% hydrogen peroxide, blocked with normal animal serum, and subsequently left incubating overnight at 4° C. with primary antibodies. The primary antibodies used are listed in the Table 2. Tissue sections were then incubated with biotinylated secondary antibody corresponding to the animal species of the primary antibody (BA-1000; Vector Laboratories, Burlingame, Calif.) and exposed to 3,3′-diaminobenzidine (SK-4105; Vector Laboratories) to visualize the peroxidase activity. Counterstaining was performed with Richard-Allan Scientific Signature Series Hematoxylin (Thermo Scientific, Waltham, Mass.). For quantification, immunoreactivities of nuclear and cytoplasmic HNF4α were independently graded by two liver pathologists, with 1,000 hepatocytes in three high-power fields being counted per sample. Normal livers (n=2), Child-Pugh B (n=4), and Child-Pugh C (n=2) were included for these analyses. The Child-Pugh B and C are grouped as cirrhotic human liver and the results are expressed as percentage over the total number of cells counted.

TABLE 2

| | | Immunohisto- | Western | |
|---|---|---|---|---|
| | | chemistry | blot | Company/ |
| Antibody/Target | Specie | Dilution | Dilution | Catalog Number |
| HNF4α | Mouse | 1:200 | 1:1000 | Abcam/Ab41898 |
| Acetyl- | Rabbit | — | 1:500 | Cusabio/CSB- |
| HNF4α(Lys106) | | | | PA727840 |
| cMET | Rabbit | 1:200 | 1:1000 | Cell |
| | | | | Signaling/8198T |
| Total AKT | Rabbit | — | 1:1000 | Cell |
| | | | | Signaling/4691S |
| Phospho- | Rabbit | 1:50 | 1:1000 | Cell |
| AKT(Ser473) | | | | Signaling/4060S |
| Phospho- | Rabbit | 1:100 | 1:1000 | Cell |
| AKT(Thr308) | | | | Signaling/9275S |
| Total AMPKα | Rabbit | — | 1:1000 | Cell |
| | | | | Signaling/5831S |
| Phospho- | Rabbit | 1:100 | 1:1000 | Cell |
| AMPKα(Thr172) | | | | Signaling/2535S |
| Total EGFR | Rabbit | — | 1:1000 | Cell |
| | | | | Signaling/4267S |
| Phospho- | Rabbit | — | 1:1000 | Cell |
| EGFR(Y1086) | | | | Signaling/2220S |
| Phospho- | Rabbit | 1:200 | 1:1000 | Cell |
| H3(Ser10) | | | | Signaling/9701S |
| Caspase 3 active | Rabbit | 1:100 | 1:500 | Abcam/Ab32042 |
| HDAC1 | Rabbit | — | 1:100 | Cell |
| | | | | Signaling/2062 |
| Histone H3 | Mouse | — | 1:100 | Santa |
| | | | | Cruz/SC517576 |
| B-Actin | Rabbit | — | 1:2000 | Cell |
| | | | | Signaling/4970S |

Protein extraction and Western blotting. To perform the protein expression analysis, the isolated hepatocytes were divided in two fractions; one fraction was used for total protein extraction, according to standard procedures previously described (Bell A W et al., 2006) and the other fraction was used for nuclear protein isolation. For nuclear protein isolation, between 1×10⁷ and 5×10⁷ isolated hepatocytes per patient were washed and harvested in 40 mmol/L Tris (pH 7.6), 14 mmol/L NaCl, and 1 mmol/L EDTA, then centrifuged (5 min, 100g). Cell pellets were suspended in 2 mL of hypotonic buffer [10 mmol/L HEPES (pH 7.9), 10 mmol/L NaH₂PO4, 1.5 mmol/L MgCl₂, 1 mmol/L DTT, 0.5 mmol/L spermidine, and 1 mol/L NaF with protease and phosphatase inhibitor cocktails (Sigma, St. Louis, Mo.)]. Following 10 minutes of incubation on ice, samples were homogenized in a Dounce homogenizer and then centrifuged (5 min, 800 g). Cell lysis was monitored with trypan blue stain. Supernatants were saved as cytoplasmic extracts. The nuclei pellets were washed two additional times in the same buffer.

Nuclear proteins were extracted in 50-100 μL of hypertonic buffer [30 mmol/L HEPES (pH 7.9), 25% glycerol, 450 mmol/L NaCl, 12 mmol/L MgCl₂, 1 mmol/L DTT, and 0.1 mmol/L EDTA with protease and phosphatase inhibitor cocktails (Sigma, St. Louis, Mo.)] for 45 minutes at 4° C. with continuous agitation. Extracts were centrifuged at 30,000 g, and the supernatants were collected and dialyzed for 2 hours against the same solution but containing 150 mmol/L NaCl. Protein concentration was determined by the Bicinchoninic Acid assay (Sigma, St. Louis, Mo.).

Western blot analysis performed according to standard procedures (Natarajan A et al., 2007). The intensity of each protein band was quantitated using National Institutes of Health Image J software. The primary antibodies and its dilutions are listed in the above shown Table 2.

RNA-Sequencing and analysis. Whole-genome strand-specific RNA-seq was used to profile RNA expression levels from human isolated primary hepatocytes. RNA-Seq libraries were prepared as described previously (Hainer S J et al., Genes Dev. 2015) and in the literature (Kumar R et al., 2012). RNA was extracted from intestinal cells using TRIzol followed by column purification (Zymo RNA clean and concentrator column) following the manufacturers' instructions. Total RNA was depleted of rRNA using pooled antisense oligo hybridization and depletion through RNaseH digestion as previously described (Morlan J D et al., 2012; Adiconis X et al., 2013). Following purification over a Zymo RNA clean and concentrator column, first strand cDNA was synthesized. Subsequently, second strand cDNA was synthesized, purified, and fragmented. RNA-seq libraries were prepared using Illumina technology. Briefly, end repair, A-tailing, and barcoded adapter ligation followed by PCR amplification and size selection. The integrity of the libraries was confirmed by quBit quantification, fragment analyzer size distribution assessment, and Sanger sequencing of ˜10 fragments from each library. Libraries were sequenced using paired-end Illumina sequencing.

Paired-end reads were aligned to hg38 using QIAGEN' s CLC Genomics workbench and were assessed as transcript per million (TPM). To sort the data, K-means clustering was performed using Cluster 3.0 (De Hoon M J et al., 2004) and heatmaps were generated using Java TreeView (Saldanha A J, 2004). The default settings for mismatch (Murphy S L et al., 2015), insertion cost (Goldman L et al., 2016) were deletion cost (Goldman L et al., 2016) were used. Ingenuity pathway analysis (IPA) was used to identify differentially expressed genes, predict downstream effects, and identify targets (QIAGEN Bioinformatics; www.qiagen.com/ingenuity). Regulatory effects analysis within IPA was used to identify the relationships between upstream regulators and biological functions. The default setting was used in the analysis (i.e., the upstream regulators were limited to genes, RNA, and proteins). The RNA-seq data are available at Gene Expression Omnibus (accession number www.ncbi.nlm.nih.gov/geo/query/acc.cgi?acc=GSE134422).

AKT Inhibition in Normal Human Hepatocytes. Normal human hepatocytes (1 million cells/well) were cultured on collagen-coated wells. Cells were culture for 6 hours in the absence of growth factors or serum. Cells then were treated with 5 μM of MK-2206 (Cayman Chemical, Ann Arbor, Mich.), an AKT inhibitor, for 24 hours. Total, cytoplasmatic, and nuclear protein were extracted for western blotting as described previously.

Statistical analysis. Data were expressed as mean±SD. Results from western blot for two statistical groups were evaluated by Mann-Whitney non-parametric test and for three statistical groups by Kruskal Wallis non-parametric test. The comparisons among groups were performed by Dunn multiple comparisons test. The association among the analyzed proteins was evaluated using the Spearman's rank correlation test. Linear regression was used to describe the relation between protein expression and clinical status measured as Child-Pugh score and MELD score. Statistics was performed using Prism 4.0 (GraphPad Software Inc., San Diego, Calif., USA). Differences were considered significant when P<0.05.

To identify the direct dependence between the proteins analyzed by western blot, the path analysis (structural equation model) was used. The path analysis model has the objective to explain a possible causal association between the observed correlations among a dependent variable and multiple independent variables. The path model was tested and modified by adding and removing a path based on the research framework and the results of regression weights and model fit. The results are plotted as diagrams that show the direct and indirect effects of the variables on the study system. The degree of correlation and the linear relation between variables is determined by a P<0.05 and an arbitrary coefficient that shows the level of importance (larger number represent a larger relation). The path analysis was performed using InfoStat version 2013 (Grupo InfoStat, FCA, Universidad Nacional de Cordoba, Cordoba, Argentina).

Unsupervised Multivariate Principal Component Analysis (PCA) was applied to western blot data to reveal the group of proteins that distinguish among the sample's clinical status. Scatter plots of the principal components (PC) that explain most of the variance were drawn. The statistical software JMP version 14 (SAS Institute, Cary, N.C., USA) was used for PCA analysis.

### Example 2. HNF4α Nuclear Localization is Decreased in Human Livers with End-Stage Liver Failure While Cytoplasmic Localization is Increased

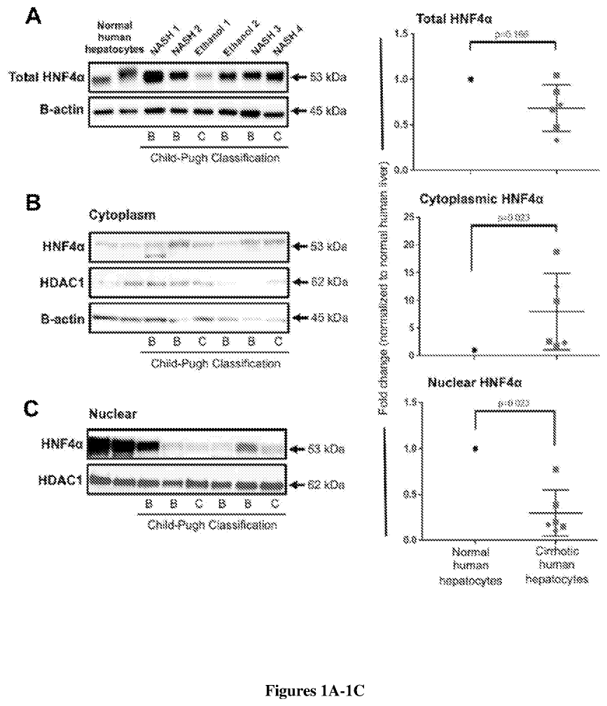

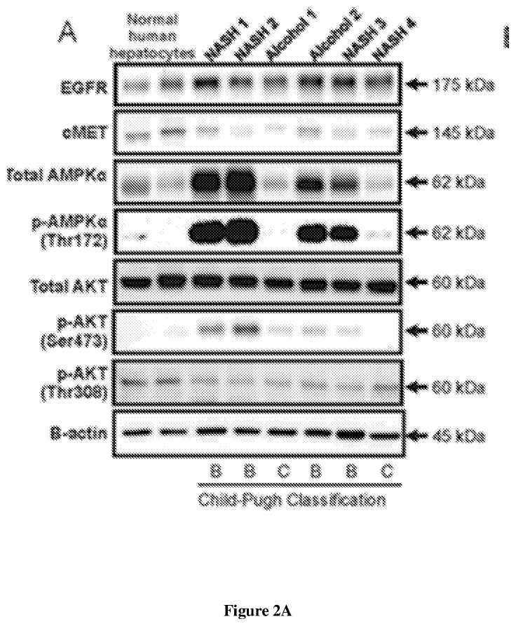

HNF4α functions as a transcription factor and nuclear localization is required for activity (Babeu J P et al., 2014; Chellappa K et al., 2012; Guo H, 2014; Hong Y H et al., 2003; Lu H et al., 2016, Song Y et al., 2015; Soutoglou E et al., 2000; Sun K et al., 2007; Yokoyama A et al., 2011; Zhou W et al., 2012; Bell A W et al., 2006; Kritis A A et al., 1996; Tanaka T et al., 2006; Walesky C et al., 2015). Therefore, immunohistochemistry and western blot on hepatocytes from diseased liver specimens were performed to determine the location of HNF4α and to correlate expression with hepatic decompensation. About 78% of hepatocytes from livers with terminal liver failure showed both only cytoplasm expression or cytoplasm and weak nuclear expression of HNF4α, whereas normal human livers displayed 75% of hepatocytes with strong nuclear localization of HNF4α. Total HNF4α protein expression from isolated hepatocytes, as assessed by western blot, did not show any statistical difference between end-stage livers and normal controls (P=0.166; FIG. 1A). This result was not surprising since the ability to discern a difference in HNF4α expression in livers from patients with degenerative disease and controls based on degree of functional decompensation required the study of a large cohort of patients (Guzman-Lepe J et al., 2018). However, in this study, a statistically significant difference was observed based on HNF4α location. HNF4α was detected at high levels in the cytoplasm (P=0.023; FIG. 1B), and at low levels in the nucleus (P=0.023; FIG. 1C) of hepatocytes isolated from functionally decompensated livers when compared to hepatocytes isolated from normal controls.

As HNF4α function and stability is regulated by a number of post-translational modifiers (Chellappa K et al., 2012; Guo H, 2014; Hong Y H et al., 2003; Lu H et al., 2016, Song Y et al., 2015; Soutoglou E et al., 2000; Sun K et al., 2007; Yokoyama A et al., 2011; Zhou W et al., 2012), and since its nuclear localization is critical to its activity, an in silico analysis was performed to evaluate which modifiers regulate HNF4α localization. It was found that AMPKα activation controls HNF4α transcription (Hong Y H et al., 2003). In addition, acetylation of HNF4α, which can be mediated by the AKT pathway, stabilizes the molecule, favoring its retention in the nucleus (Soutoglou E et al., 2000) (FIG. 5).

### Example 3: HNF4α is the Major Regulator of Human Hepatocyte Function in Advanced Liver Disease

Assessment was next performed to compare gene expression differences between human hepatocytes from normal controls (n=4) and those recovered from patients with cirrhosis and terminal liver failure (n=4) (Child-Pugh C), limiting the study to patients with NASH and alcohol-mediated Laennec's cirrhosis. Hierarchical clustering of RNA-seq data revealed three major dynamic patterns associated with cirrhosis and liver dysfunction, a calculated in the heatmap of a K-means clustering (loge FC over controls; K=3). Cluster I (3478 genes) and III (1669 genes) represented genes that were moderately to highly up-regulated in hepatocytes from patients with terminal liver failure relative to control human hepatocytes. Most genes in these clusters were related to autophagy and apoptotic signaling (data not shown).

Cluster II, however, consisted of 1669 genes that were significantly down-regulated in end-stage hepatocytes, and included genes encoding the serine-threonine protein kinase (AKT1), cytochrome P450s (cytochrome P450 [CYP]c8, CYP2c9, CYP2e1, CYP3A4), and hepatocyte nuclear factors (HNF4α, forkhead box a1 [FOXa1]). The top pathways represented in this cluster included farnesoid X receptor/retinoid X receptor (RXR) and liver X receptor/RXR activation, mitochondrial dysfunction, oxidative phosphorylation, and inhibition of RXR function. Pathway analysis on these down-regulated genes showed that HNF4α was a central upstream regulator, and the heatmap confirmed many similarities in the gene expression profile of hepatocytes from patients with NASH and alcohol-mediated Laennec's cirrhosis(data not shown). These results were nearly identical to the gene expression profile of rat hepatocytes that were recovered from cirrhotic livers with terminal failure (Liu L et al., 2012).

### Example 4: cMET and AKT Phosphorylation Correlates with HNF4α Nuclear Localization in Human Hepatocytes from Patients with End-Stage Liver Failure

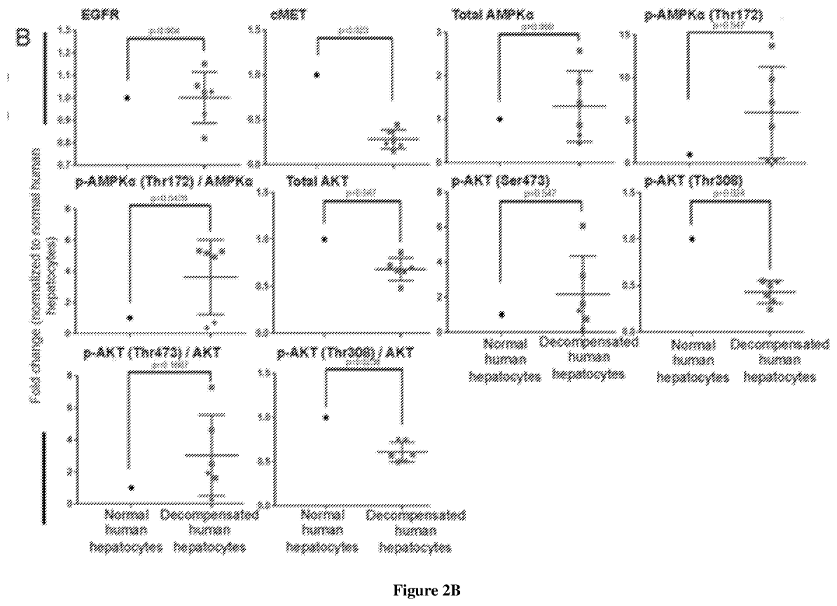

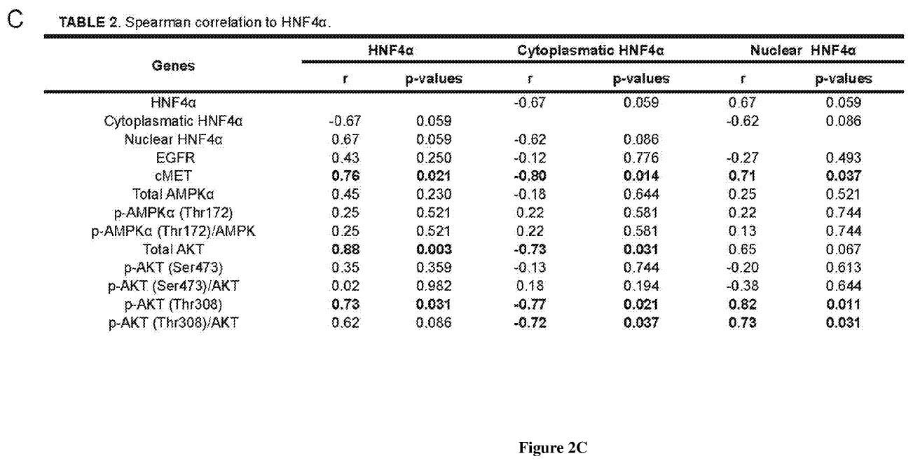

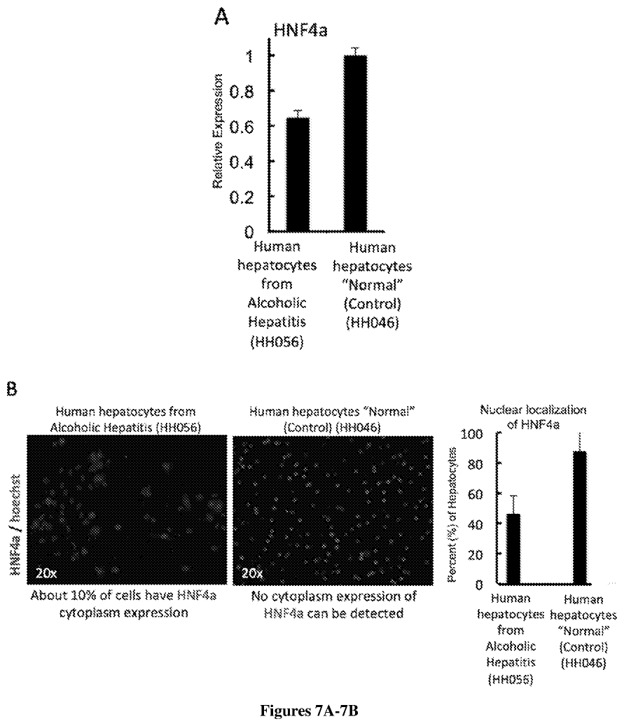

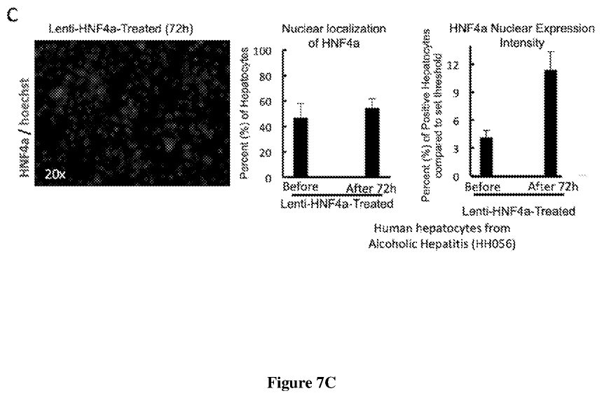

As EGFR and cMET have been shown to regulate the AMPK and AKT pathways (Komposch K et al., 2015; Paranjpe S et at, 2016; Tsagianni A et al, 2018) that were identified in the in silico and RNAseq analysis as important modulators of HNF4α, antibody-based assays for these molecules in liver specimens were performed. cMET expression in decompensated liver specimens was markedly reduced when measured by both immunohistochemistry and western blot (P=0.023; FIGS. 2A and 2B) when compared to isolated control human hepatocytes. There was no significant difference in the EGFR expression between normal and diseased liver specimens (FIGS. 2A and 2B). However, the active form of EGFR, phospho-EGFR(Y1086), was highly expressed in hepatocytes derived from diseased livers from patients with decompensated disease when compared to normal human hepatocytes (FIGS. 6A and 6B). In addition, as continuous cycles of cell death and hepatocyte replication are hallmarks of cirrhosis (Tsochatzis E A et al., 2014), this observation in hepatocytes from patients was corroborated with terminal liver failure, demonstrating expression of replicative [phospho-H3(Ser10)] and cell death (active caspase 3) markers in end-stage hepatocytes using immunohistochemistry and western blot.

Since cMET can control AMPKa and AKT and cMET significantly downregulated in hepatocytes from functionally decompensated livers, the activation processes for AMPKα and AKT was analyzed. Total AMPKα, activated AMPKα(Thr172) and its ratio did not differ statistically between hepatocytes with decompensated function and normal human hepatocytes (FIGS. 2A and 2B). However, total AKT, activated AKT(Thr308), and its ratio decreased significantly in liver specimens and isolated hepatocytes from patients with decompensated hepatic function (FIGS. 2A and 2B). Another AKT phosphorylation site (Ser473) was unchanged in liver specimens or isolated hepatocytes from normal controls or decompensated specimens (FIGS. 2A and B).

To further analyze the relationship between HNF4α, it's nuclear localization, and post-translation modulation, a Spearman's rank correlation test was performed. cMET expression showed a positive, and statistically significant correlation with total HNF4α (r=0.76; P=0.021; FIG. 2C) and nuclear HNF4α (r=0.71; P=0.037; FIG. 2C). Activated AKT (Thr308) also showed a positive, and statistically significant correlation with total HNF4α (r=0.73; P=0.031; FIG. 2C) and nuclear HNF4α (r=0.82; P=0.011; FIG. 2C), whereas cytoplasmic HNF4α correlated negatively with cMET (r=−0.80; P=0.014; FIG. 2C) and activated AKT(Thr308) (r=−0.77; P=0.021; FIG. 2C). In addition, the ratio of phospho-AKT(Thr308)/total AKT correlated positively with cMET (r=0.80; P=0.014; FIG. 6A) and total AKT (r=0.71; P=0.037; FIG. 6A). Thus, reduced cMET was associated with reduced activation of the AKT pathway, reduced HNF4α in the nucleus, and more expression of HNF4α in the cytoplasm.

### Example 5: Nuclear Localization of HNF4α is Affected by the cMET/AKT Axis and Aorrelates with Extent of Liver Dysfunction

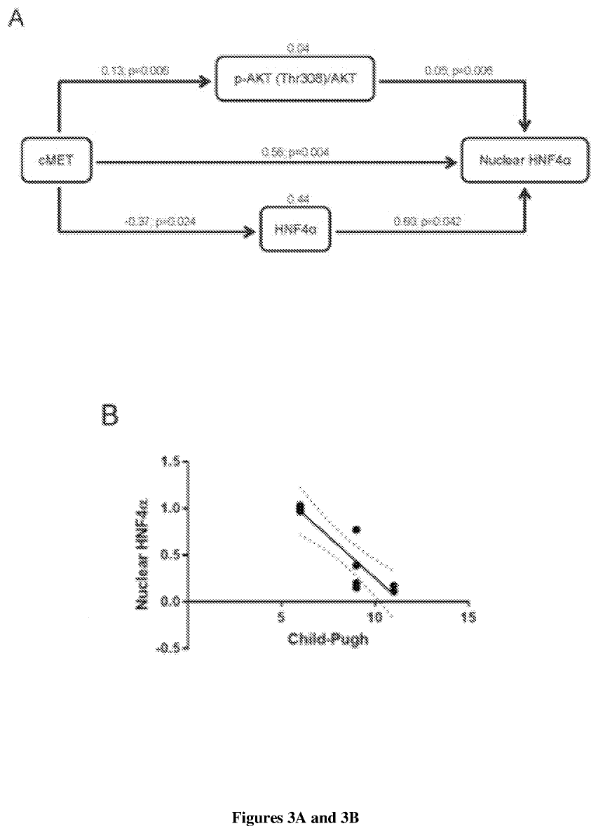

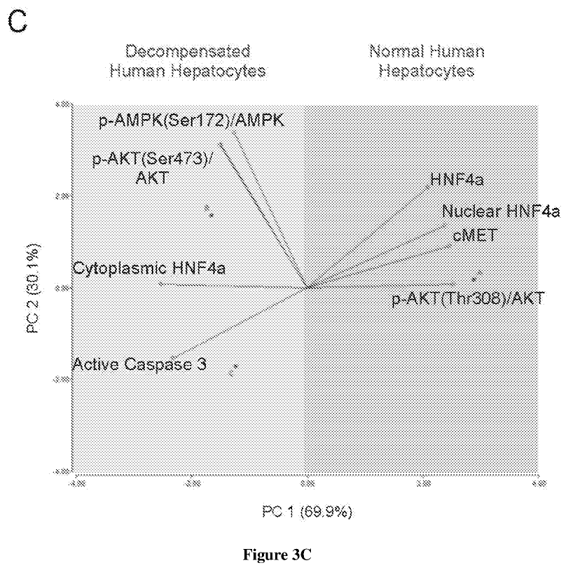

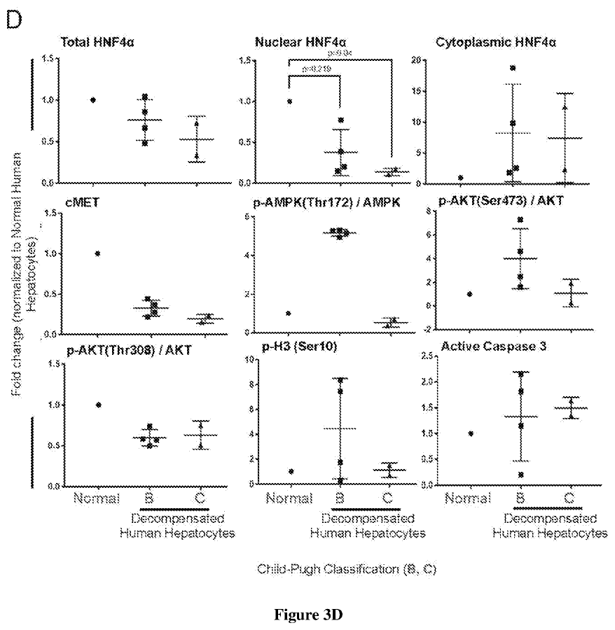

As seen in FIG. 3A, pathway analysis revealed a significant causal relationship between cMET expression and nuclear HNF4α expression (0.56; P=0.004) and a direct relationship between levels of nuclear HNF4α and the activated AKT(Thr308)/total AKT ratio (0.05; P=0.006). Modeling also demonstrated that total HNF4α expression levels contribute to nuclear localization (0.60; P=0.042). However, cMET expression was negatively associated with total HNF4α expression (−0.37; P=0.024) (FIG. 3A) indicating that cMET expression does not directly affect total HNF4α expression but only its nuclear localization. To assess whether the levels of nuclear expression correlate with extent of liver dysfunction (Child-Pugh Score), a linear regression analysis was performed, which showed that nuclear HNF4α expression levels have a significant inverse relationship with the Child-Pugh score (R²=0.80; P=0.007) (FIG. 3B). Together, these pathway and linear regression statistical analyses of protein expression show that HNF4α localization is associated with hepatic disease progression and that cMET expression and AKT phosphorylation play a central role in maintaining hepatocyte HNF4α nuclear localization and function.

Next, principal component analysis (PCA) was performed to assess HNF4α post-translation modifier-related molecules, and to delineate if a pattern of molecules correlates with hepatic function in end-stage hepatocytes (Child-Pugh score) (FIGS. 3C and 3D). As shown in FIG. 3C, PC1 (69.9%) and PC2 (30.1%) discriminate 100% of the variability in level of hepatic function (Child-Pugh score) in the isolated hepatocytes studied. The vectors that characterized normal human hepatocytes were cMET and activated AKT(Thr308)/total AKT ratio, total HNF4α, and nuclear HNF4α whereas the negative characteristics, which characterized failing human hepatocytes were cytoplasmic HNF4α and active caspase 3 expression (FIGS. 3C and 3D). Together, this statistical analysis corroborates the molecular profiling of human hepatocytes with terminal liver failure and establishes a causal connection between the expression of cMET, activated AKT(Thr308), and total and nuclear HNF4α.

### Example 6: Retention of HNF4α in the Nucleus is Reduced in Patients with End-Stage Liver Failure Through Decreased Acetylation

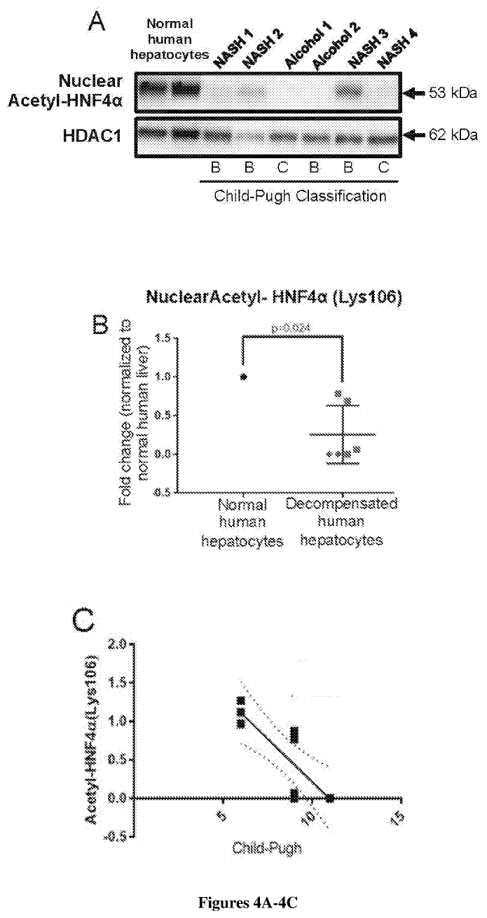

One of the targets of AKT is activation of CREB binding protein (Dekker F J et al., 2009). It is well known that CREB-binding protein has an intrinsic acetylation activity on nucleosomal histones, which increase the access of transcription factors to nucleosomal DNA, and thus, activate transcription and retention of transcription factors in nuclei. Thus, this axis can be related to HNF4α nuclear retention (Soutoglou E et al., 2000). As genome wide transcriptome analysis and the in silico analysis indicated that the cMET/AKT kinase axis pathway can control HNF4α localization and stability through the activation of CREB binding protein (Kumar R et al., 2012), nuclear expression of acetylatedHNF4α was measured (FIGS. 4A-4C), and finding that HNF4α acetylation in the nucleus was significantly reduced in hepatocytes from patients with terminal liver failure when compared to normal controls (P=0.024; FIG. 4A). And to confirm that acetylation of HNF4α relates to the extent of liver dysfunction (Child-Pugh Score), a linear regression analysis was performed, and showed that a decrease in acetylated HNF4α correlates directly and significantly with liver dysfunction (R²=0.71; P=0.004; FIG. 4C). As a proof of principle, preliminary experiments were conducted in freshly isolated normal human hepatocytes by inhibiting AKT signaling to corroborate the role of activated-AKT(Thr308) in HNF4α nuclear localization. Freshly isolated normal human hepatocytes were treated with MK-2206, a potent allosteric pan-AKT inhibitor. After 24 hours of AKT-inhibitory treatment, 80% of activated AKT(Thr308), 25% of nuclear HNF4α, and 14% of acetylated nuclear HNF4α expression were reduced when compared with the nontreated controls.

Example 7: Retention of HNF4α in the Nucleus is Regulated by Multiple Signaling Molecules, Showing Significantly Negative Association with End-Stage Liver Failure

HNF4α is the master regulator of liver functions (Babeu J P et al., 2014; Chellappa K et al., 2012; Guo H et al., 2014; Lu H et al., 2016, Song Y et al., 2015; Soutoglou E et al., 2000; Sun K et al., 2007; Xu Z et al., 2007; Zhou W et al., 2012; Bell A W et al., 2006). Alterations in HNF4α expression has related to liver diseases with multiple etiologies such as cancer, hepatitis B and C, alcohol-mediated cirrhosis, and NASH (Babeu J P et al., 2014; Chellappa K et al., 2012; Guo H et al., 2014; Lu H et al., 2016, Song Y et al., 2015; Soutoglou E et al., 2000; Sun K et al., 2007; Xu Z et al., 2007; Zhou W et al., 2012; Bell A W et al., 2006). In animal models with TLF a strong reduction in HNF4α expression was identified and by restoring production of HNF4α using gene therapy reboots the liver cells to normal function (Nishikawa T et al., 2014). To assess whether this observation applies to human, a study was done on liver-enriched transcription factor expression in the livers of a large cohort of patients with decompensated liver function (Guzman-Lepe J et al., 2018). It was found that HNF4α mRNA levels were down regulated and correlated with the extent of liver dysfunction based upon Child-Pugh classification. Nuclear localization of HNF4α in those studies was not uniform (Guzman-Lepe J et al., 2018).

Human hepatocytes were isolated from the explanted livers of patients with cirrhosis and end-stage (Child-Pugh B, C) liver failure caused by NASH and alcohol-mediated Laennec's cirrhosis. In these isolated hepatocytes there was an increase in HNF4α located in the cytoplasm and a decrease in HNF4α in the nucleus compared to that seen in control normal hepatocytes. In addition, localization of HNF4α to the cytoplasm or nucleus in human failing cirrhotic hepatocytes correlated with degree of hepatocyte dysfunction. These data indicate that pathways regulating HNF4α nuclear transport or retention can be targets for the treatment of terminal liver failure.

AMPK and AKT kinases are the main components that can regulate HNF4α localization (Hong Y H et al., 2003; Song Y et al., 2015; Soutoglou E et al., 2000). AMPK plays a central role in maintaining energy homeostasis, promoting adenosine triphosphate (ATP) production pathways and reducing ATP consumption (Woods A et al., 2017), while AKT activation promotes cell proliferation, survival, and growth (Manning B D et al., 2017; Morales-Ruiz M et al., 2017). AKT activation is mediated by phosphorylation at threonine 308 and/or serine 473(Praveen P et al., 2016; Inoue J et al., 2017). What is disclosed herein shows a significant correlation between activated AKT(Thr308) and HNF4α localization since activated AKT(Thr308) levels were significantly decreased in end-stage human hepatocytes. These findings indicate that AKT phosphorylation at Thr308 can play an important role in hepatocyte failure in terminal stages of liver disease.

Analysis was done on cMET and EGFR, the two central receptors that are upstream regulators of AKT and AMPK and are related to liver function and regeneration (Natarajan A et al., 2007; Komposch K et al., 2015; Paranjpe S et al., 2016; Tsagianni A et at, 2018; Alam A et al., 2017). Combined disruption of cMET and EGFR in mice alters liver homeostasis and leads to terminal liver failure (Tsagianni A et al., 2018). In human failing cirrhotic hepatocytes, there was reduced expression of cMET and decreased expression correlated directly with HNF4α localization. In contrast, there was no difference in total EGFR expression in either human failing cirrhotic hepatocytes or control human hepatocytes. Thus, in human liver, cMET can play an important role in regulating AKT pathway activation and HNF4α localization and function. Moreover, using different statistical analyses (Spearman's rank correlation test, pathway analysis, linear regression analysis and principal component analysis) enables the establishment of the association between the post-translational modifiers analyzed in human failing cirrhotic and normal hepatocytes. These statistical analyses revealed that cMET and activated AKT(Thr308) were directly related to the expression levels of nuclear HNF4α. The negative association found between cMET protein expression and total HNF4α indicates that cMET expression does not directly affect total HNF4α expression but only its nuclear localization.

The acetylation of HNF4α can be affected in the cirrhotic failing hepatocytes, based on the low expression levels of activated AKT(Thr308) in human failing cirrhotic hepatocytes and the direct effect of AKT on CREB binding protein, a molecule which has an intrinsic acetylation activity that increases transcription factor binding to nucleosomal DNA. Indeed, nuclear HNF4α acetylation was strikingly reduced in human hepatocytes from cirrhotic livers and its level was associated with degree of hepatic dysfunction. These observations indicate that AKT phosphorylation at threonine 308 mediates the HNF4α nuclear retention through acetylation by controlling CREB binding protein (Soutoglou E et al., 2000).

In summary, localization of HNF4α in the cytoplasm results from alterations of the molecular pathways, which maintain HNF4α in the nucleus during advanced stages of liver disease. cMET and activated AKT(Thr308) are downregulated and affect acetylation and nuclear retention of HNF4α. These data shows the restoration of hepatocyte function in chronic liver diseases through localizing HNF4α to the nucleus.

### Example 8: Transduction of Primary Human Hepatocytes with Transcription Factor Lentivirus (LV) Constructs.

Transduction of primary human hepatocytes with transcription factor LV. The hepatocytes are cultured in a double collagen (thick layers) system to prevent dedifferentiation of hepatocytes. Collagen sandwich protocol is used subsequently. Prepare the following: WARM: dPBS, HMM (Basal+SingleQuots), HCM (HBM Basal+HCM SingleQuots); ON ICE: Green Fluorescent Protein (GFP) LV*, Transcription Factor (TF) LV*, Max Enhancer, TransDux; OTHERS: 1.5 mL tubes, 50 mL tubes, tips, pipettes.

1. Plate 5e5 hepatocytes per well onto thick layer collagen and let cells attach for 4 hours.

2. Wash wells with warm dPBS 2× to remove dead cells and replace media with 500 uL HMM (no FBS).

3. Prepare 5 tubes and label as: a. GFPLV-2; b. GFPLV-10; c. TFLV-0; d. TFLV-2; e. TFLV-10.

4. Prepare HMM/Max Enhancer/TransDux (HMT) solution in a 50 mL tube: 12.323 mL HMM+3.100 mL Max Enhancer+77.5 uL TransDux.

5. Prepare LV solutions in pre-labeled tubes: a. GFPLV-2: 618.3 uL HMM+1.7 uL GFP LV; b. GFPLV-10: 611.6 uL HMM+8.4 uL GFP LV; c. TFLV-0: 620 uL HMM only; d. TFLV-2: 618.6 uL HMM+1.44 uL TF4 LV; e. TFLV-10: 612.82 uL HMM+7.18 uL TF4 LV.

6. Replace HMM media in wells with 500 μL HMT solution.

7. Dispense 100 uL of each LV solution into wells. Swirl plates to mix.

8. The next day, wash wells with warm dPBS 2× to remove dead cells and remaining LV solution.

9. Overlay the cells with thick collagen and let the collagen gelify for 2 hours.

10. Add 500 uL HCM to the wells and replace with fresh HCM every day.

11. Obtain samples at 72 and 96 hours post transduction (refer to protocols at 72 hours).

12. For samples to be obtained at 96 hours, wash cells with warm dPBS and replace media with 500 uL HCM (no FBS).

Notes: *Quickly thaw LV in water bath at 37° C. Transfer to the hood, mix by rotation, inversion, or gentle vortexing and keep on ice. Unused LV can be aliquoted and refrozen at −80° C. with 10-20% loss of viral activity with each re-freeze.

Transcription Factor (TF) LV Constructs.

The TF LV Constructs are lentiviral vectors (Systems Bioscience, Cat#CS970S-1) containing a polynucleotide encoding PROX1, NR5A2, NR0B2, MTF1, SREBP1, EP300, POM121C or HNF4α or an RNAi corresponding to DNAJB1/HSP40, ATF6, ATF4 or PERK.

Collect conditioned medium for ELISA (72 and 96 hours). Collect 1200 uL of conditioned medium from 2 wells for each group and transfer into a 1.5 mL tube. Replace collected medium with warm HMM. Centrifuge conditioned medium at 20,000×g for 2 mins. Transfer supernatant into a new tube and store at −20° C.

Take photos of GFP expression and Brightfield to determine transfection efficiency (72 and 96 hours). Wash floating cells with warm dPBS. Replace with warm HCM. Take photos using the filter 2 (green excitation) for GFP expression and filter 6 for BrightField.

Collect cell lysates in Qiazol for RNA extraction (72 and 96 hours). Wash cells with warm dPBS 2×. Coat wells with 600 uL Qiazol and incubate for 1 minute. Scratch cells off the plate using a P1000 and transfer to a 1.5 mL tube. Store at −20 ° C. until RNA isolation.

Fix wells for IF (72 Hours). Wash cells with warm dPBS 2×. Coat wells with 750 uL 4% PFA solution and incubate for 40 mins. Wash wells with 1mL dPBS 3×, 10 mins per wash. Add 1 mL of dPBS. Store at 4C until staining for HNF4A.

Collect cell lysates for Western Blot (72 and 96 hours). Prepare an ice-cold lysis solution containing:

| | 1 mL | 2 mL | 3 mL |
|---|---|---|---|
| a. NP40 cell lysis buffer—90% | 900 uL | 1800 uL | 2700 uL |
| b. Complete (1 tab/1.5 mL)—6% | 60 uL | 120 uL | 180 uL |
| c. Halt protease inhibitor—2% | 20 uL | 40 uL | 60 uL |
| d. 0.1M PMSF in ethanol—2% | 20 uL | 40 uL | 60 uL |

Wash cells with warm dPBS 2×. Coat wells with 200 uL of the ice-cold lysis solution and incubate for 30 mins with rocking in the walk-in fridge. Detach cells using a rubber policeman and collect and transfer lysates including precipitates and solids into pre-labeled tubes. Spin at 20,000×g for 10 mins at 4° C. Transfer supernatant into a clean pre-labeled 1.5 mL tube. Store lysates in the −80° C. freezer.

TF Immunofluorescent Co-Staining (For 12-well plates with thick collagen sandwich layers). Aspirate samples gently to prevent detachment of cells. Do not let the samples dry. *Spin down at max speed for 5 min.* Secondary antibody may be replaced with other suitable antibodies depending on the experiment.

Fixation (If cells are already fixed, moved to Blocking and Permeabilization step). Fix the samples with 4% paraformaldehyde in PBS pH 7.4 for 40 min at room temperature. Wash samples 3× with ice cold PBS, 10 min each wash. Proceed with staining or store at 4° C. until staining (maximum 2 weeks).

Blocking and Permeabilization. Wash samples 2× with 1 mL PBS. Wash samples 3× with 1 mL Wash Buffer (PBS, 0.1% BSA, and 0.1% Tween), 10 min each wash. Block and permeabilize by incubating samples for 2 hours with 1 mL Blocking Buffer (PBS, 10% normal donkey serum, 1% BSA, 0.1% Tween, and 0.1% Triton X-100).

Antibody Incubation: Vortex and spin down* mouse anti-TF stock 1° Ab. Prepare a 1:500 mouse anti-TF 1° Ab dilution in Blocking Buffer. Coat samples with 600 uL diluted 1° Ab and incubate in a humidified chamber for 6 hours at room temperature or overnight at 4° C. Aspirate the 1° Ab solution and wash the cells 3× with Wash Buffer, 10 minutes each wash. Vortex and spin down* donkey anti-mouselgG-AF594 (Invitrogen A21203). Prepare a 1:250 diluted 2° Ab in Blocking Buffer. Coat samples with 600 μL diluted 2° Ab and incubate in a humidified chamber for 2 hours at room temperature. Aspirate the 2° Ab solution and wash the cells 3× with Wash Buffer, 10 minutes each wash.

Counter staining and Mounting: Wash samples with PBS 3×. Incubate cells for 2 min in 1 mL of 1 ug/ml Hoechst 33342 in PBS in the dark. Wash samples 3× with PBS. The sample may be stored in the dark at 4° C. Test TF using RED channel.

### Example 9: Transfection of Primary Human Hepatocytes with Transcription Factor (TF) mRNA (50, 100, 500 ng)

Transfection of primary human hepatocytes. Prepare the following: WARM: DPBS, HMM (HMM Basal Medium+HMM SingleQuots), Opti-MEM→RT; ON ICE: Lipofectamine Messenger Max→RT, mRNA→RT; OTHERS: 1.5 mL tubes, 50 mL tubes, tips, pipettes.

1. Wash cells with warm DPBS and replace media with 500 uL HMM (no FBS).

2. Prepare two sets of tubes labeled as: a. GFP-50. b. GFP-100. c. GFP-500. d. TF-0. e. TF-50. f. TF-100. g. TF-500. TF is selected from PROX1, NR5A2, NR0B2, MTF1, SREBP1, EP300, POM121C and HNF4α.

3. Prepare second diluted mRNA in Opti-MEM: a. DilGFP: 32.5 uL OptiMEM +3.61 uL GFP mRNA. b. DilHNF: 40.3 uL OptiMEM+4.47 uL TF mRNA.

4. Prepare mRNA-OptiMEM Mix in the first set of prelabeled tubes: a. GFP-50: 312.2 uL Opti-MEM+2.8 uL DilGFP. b. GFP-100: 309.4 uL Opti-MEM+5.6 uL DilGFP. c. GFP-500: 287.3 uL Opti-MEM+27.7 uL DilGFP. d. HNF-0: 315 uL Opti-MEM. e. TF-50: 311.5 uL Opti-MEM +3.5 uL DilTF. f. TF-100: 308.1 uL Opti-MEM+6.9 uL DilTF. g. TF-500: 280.7 uL Opti-MEM+34.3 uL DilTF.

5. Prepare diluted Lipo Mix: a. 2,220.75 uL Opti-MEM+141.75 uL Lipo.

6. Vortex diluted Lipo mix for 2-3 seconds and dispense 310 uL into the second set of pre-labeled tubes. Incubate at RT for 10 mins.

7. Transfer 310 uL of each mRNA-OptiMEM Mix into the tubes contain diluted Lipo Mix. Incubate at RT for 5 mins.

8. Dispense 100 uL of each mix into each well.

9. Incubate and on the next morning, wash with warm dPBS and replace with 500 uL HMM (no FBS).

10. Obtain samples at 24* and 48* hours post transfection (depends on when the hepatocytes are plated).

Collect conditioned medium for ELISA (24 and 48 hours). Collect 1200 uL of conditioned medium from 2 wells for each group and transfer into a 1.5 mL tube. Replace collected medium with warm HMM. Centrifuge conditioned medium at 20,000×g for 2 mins. Transfer supernatant into a new tube and store at −20° C.

Take photos of GFP expression and BrightField to determine transfection efficiency (24 and 48 hours). Wash floating cells with warm dPBS. Replace with warm HMM. Take photos using the filter 2 (green excitation) for GFP expression and filter 6 for BrightField.

Collect cell lysates in Qiazol for RNA extraction (24 and 48 hours). Wash cells with warm dPBS 2×. Coat wells with 600 uL Qiazol and incubate for 1 minute. Scratch cells off the plate using a P1000 and transfer to a 1.5 mL tube. Store at −20° C. until RNA isolation.

Fix wells for IF (24 hours). Wash cells with warm dPBS 2×. Coat wells with 750 uL 4% PFA solution and incubate for 20 mins. Wash wells with 1 mL dPBS 3×, 5 mins per wash. Add 1 mL of dPBS. Store at 4C until staining for TF.

Collect cell lysates for Western Blot (24 and 48 hours). Prepare an ice-cold lysis solution containing:

| | 1 mL | 2 mL | 3 mL |
|---|---|---|---|
| a. NP40 cell lysis buffer—90% | 900 uL | 1800 uL | 2700 uL |
| b. Complete (1 tab/1.5 mL)—6% | 60 uL | 120 uL | 180 uL |
| c. Halt protease inhibitor—2% | 20 uL | 40 uL | 60 uL |
| d. 0.1M PMSF in ethanol—2% | 20 uL | 40 uL | 60 uL |

Wash cells with warm dPBS 2×. Coat wells with 200 uL of the ice-cold lysis solution and incubate for 30 mins with rocking in the walk-in fridge. Detach cells using a rubber policeman and collect and transfer lysates including precipitates and solids into pre-labeled tubes. Spin at 20,000×g for 10 mins at 4° C. Transfer supernatant into a clean pre-labeled 1.5 mL tube. Store lysates in the −80° C. freezer.

Immunofluorescent co-staining (staining, fixation, blocking and permeabilization, antibody incubation, and counter staining and mounting) is the same as stated above in Example 9.

### Example 10. Transcriptional Factors and Regulators PROX1, NR5A2, NR0B2, MTF1, SREBP1, EP300 and POM121C Improve Nuclear Expression of HNF4α Cirrhotic Hepatocytes with Terminal Liver Failure

It was found that liver-enriched transcription factors are stably down regulated in hepatocytes from rats with end-stage cirrhosis, and that forced re-expression of one of them, hepatocyte nuclear factor 4 alpha (HNF4α), reprograms dysfunctional hepatocytes to regain function, both in culture and in vivo. It has been shown in a large cohort of patients with advanced liver disease that the level of HNF4α mRNA expression in the diseased liver correlated with extent of hepatic dysfunction (Child-Pugh classification) and that it's expression was not localized in the nucleus, as was the case in the rats studies. In the livers of patients with advanced cirrhosis, HNF4α RNA expression levels decrease as hepatic function deteriorates, and protein expression is found in the cytoplasm. These findings can explain impaired hepatic function in patients with degenerative liver disease. Moreover, RNA-seq analysis revealed that HNF4α and other Transcriptional Factors/regulators-related pathways that are involved in nucleus protein translocation are down-regulated in cirrhotic hepatocytes from patients with terminal failure, where, nuclear levels of HNF4α were significantly reduced, and, cytoplasmic expression of HNF4α was found to be increased. Additionally, it was found that four key Transcriptional regulators of the endoplasmic reticulum (ER) stress were significantly upregulated. This study indicates that manipulation of HNF4α and pathways involved with post-translational modifications can restore hepatocyte function in patients with terminal liver failure.

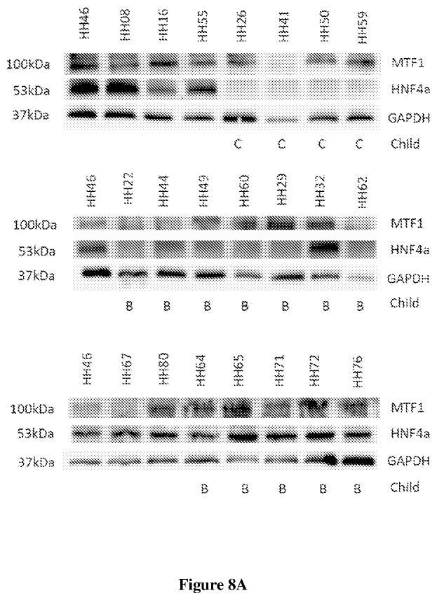

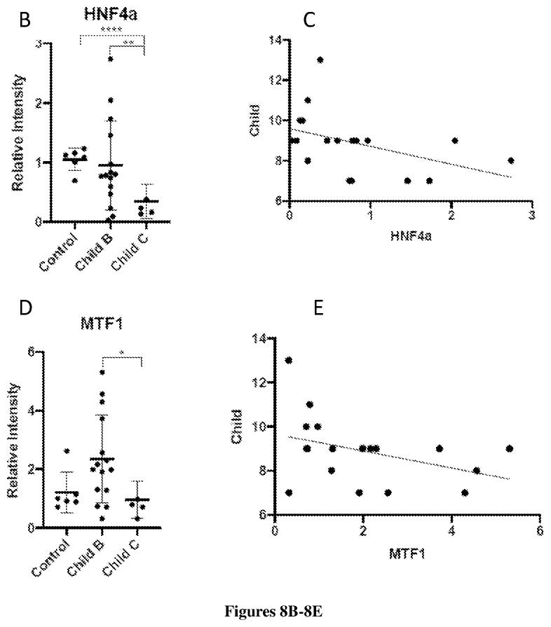

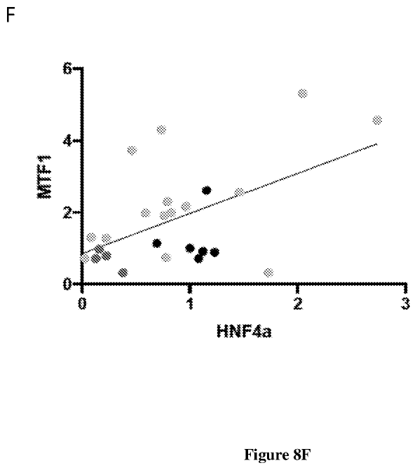

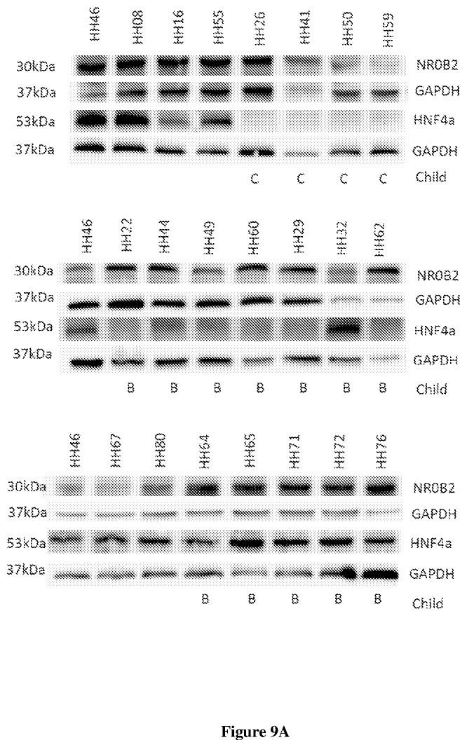

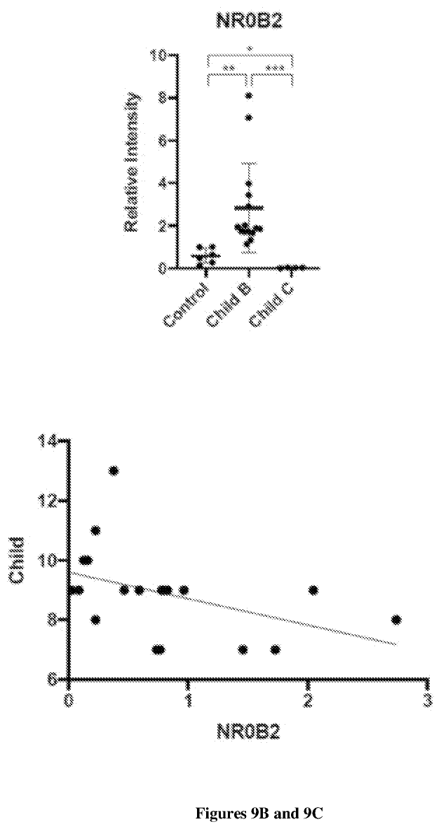

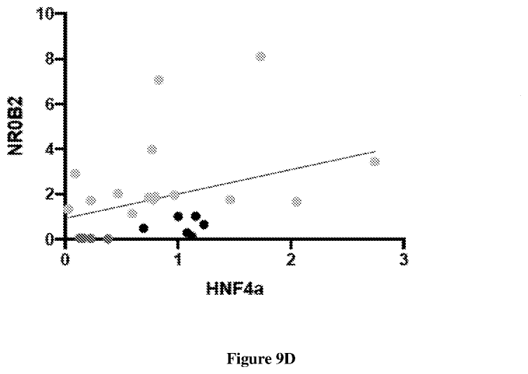

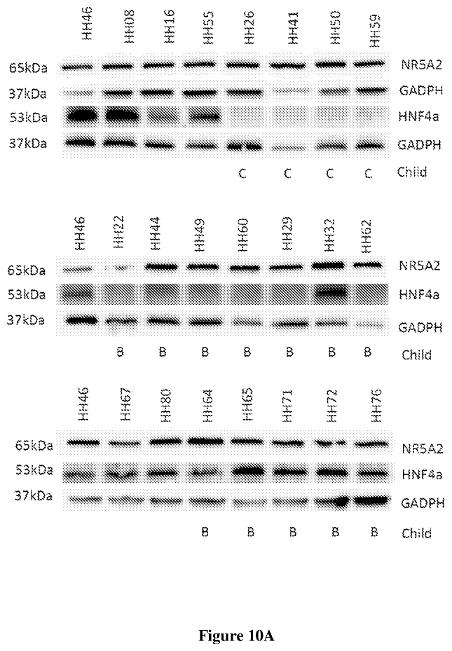

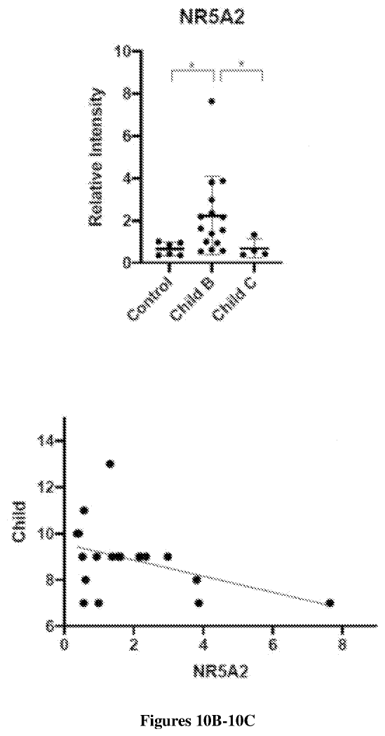

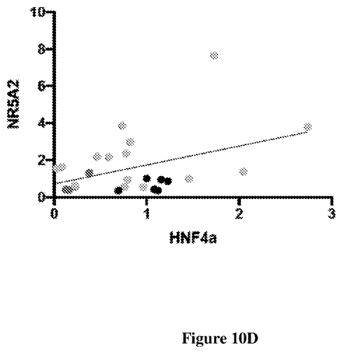

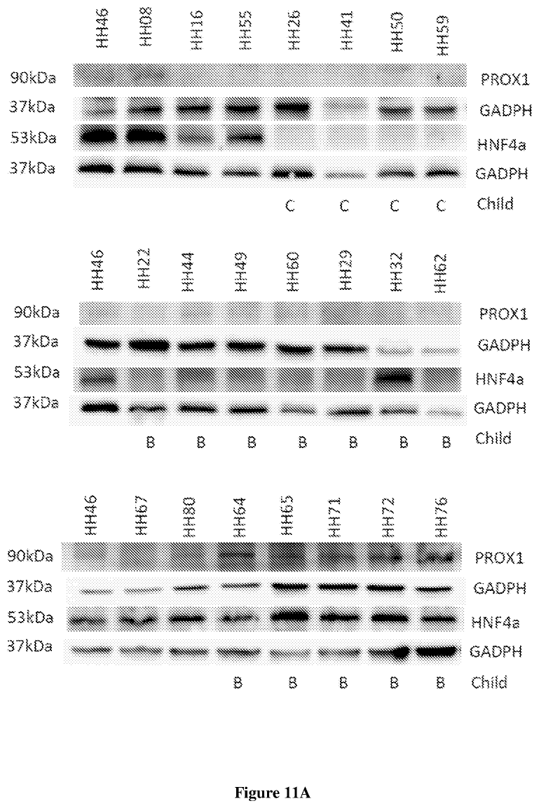

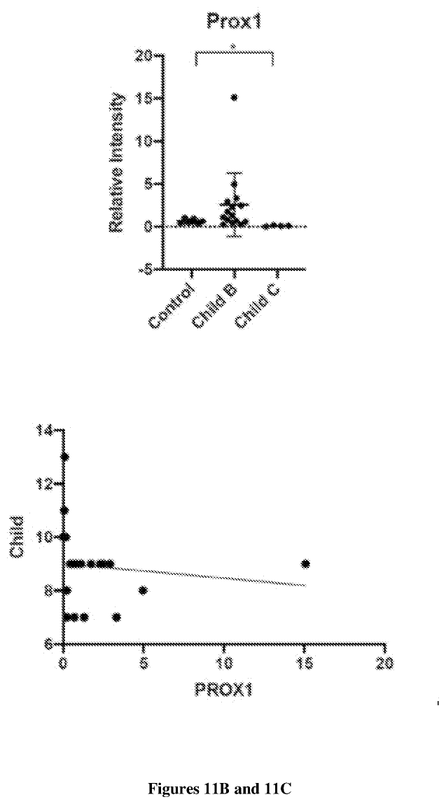

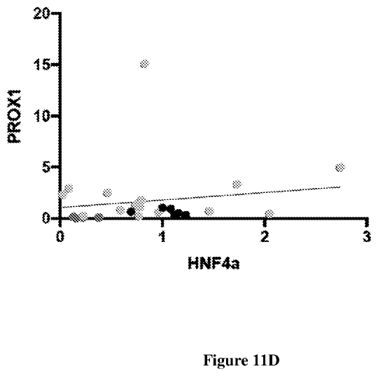

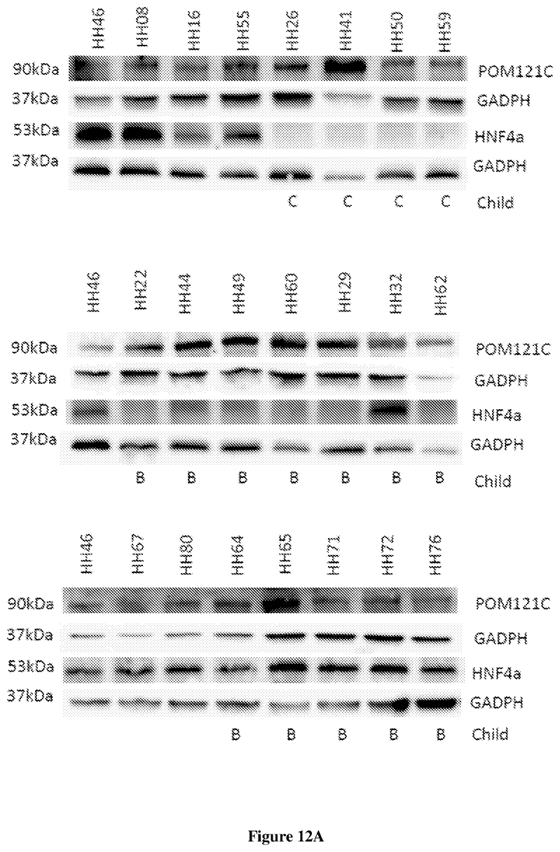

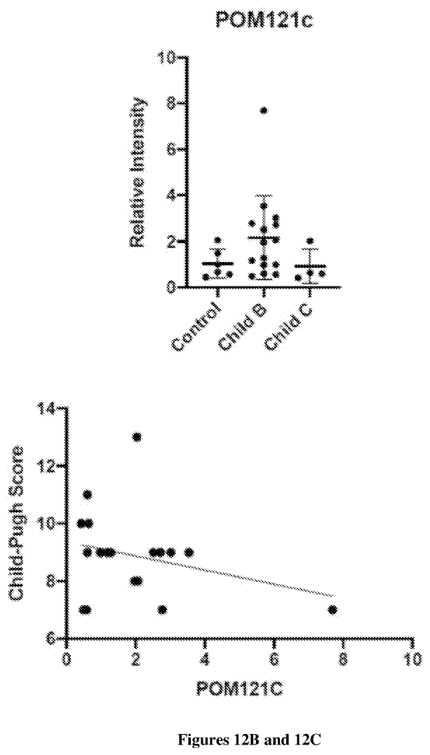

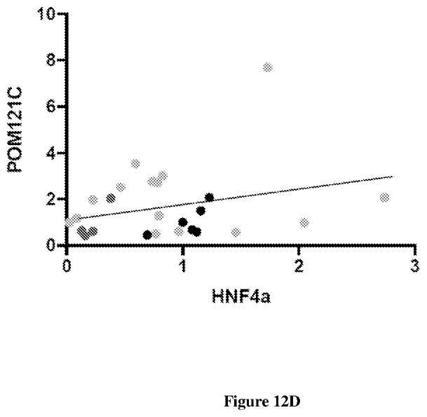

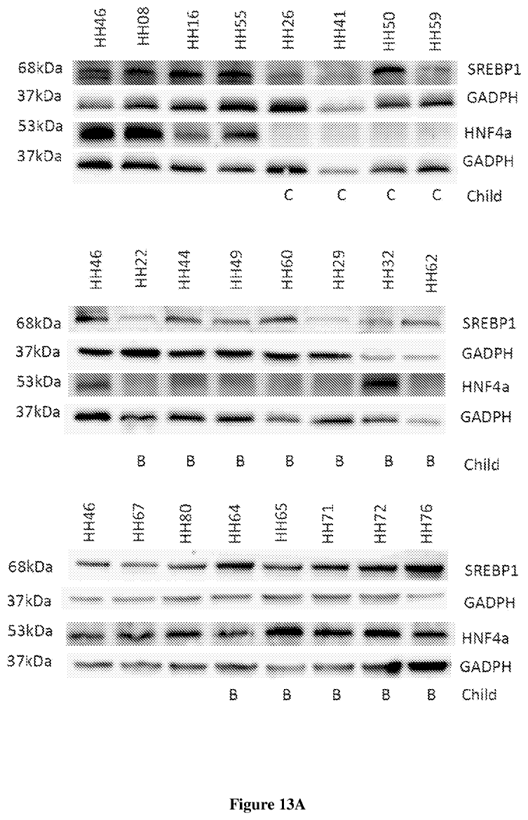

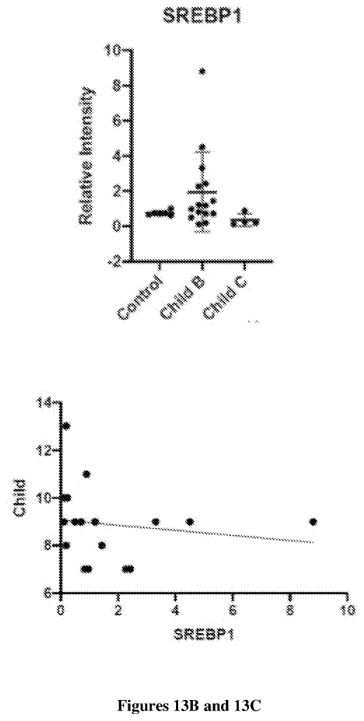

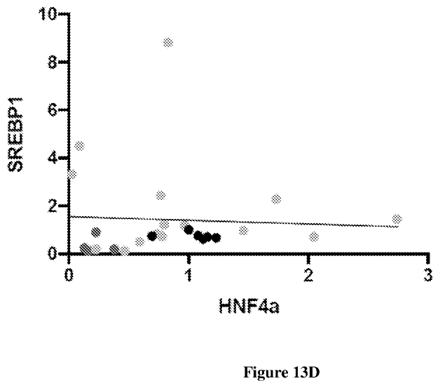

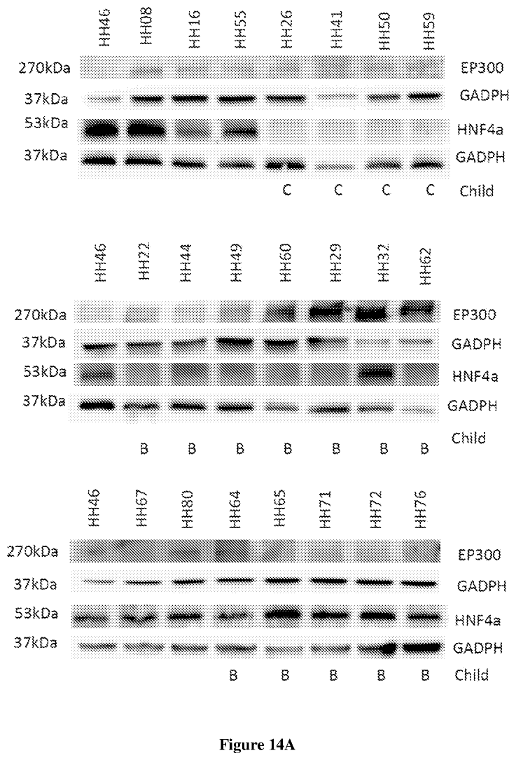

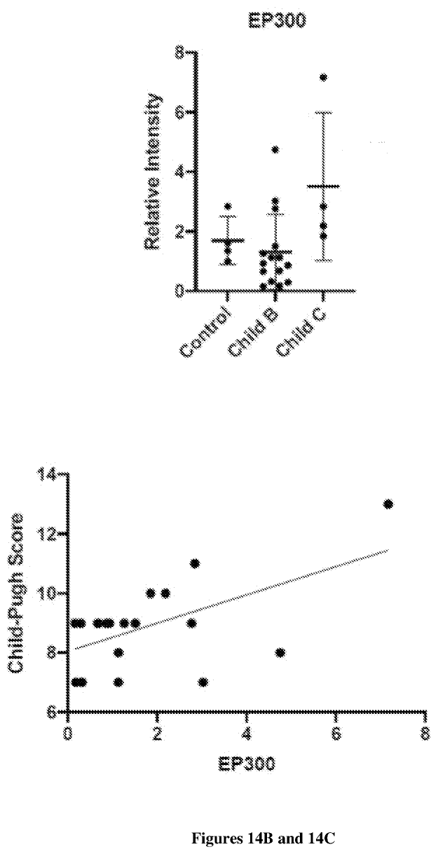

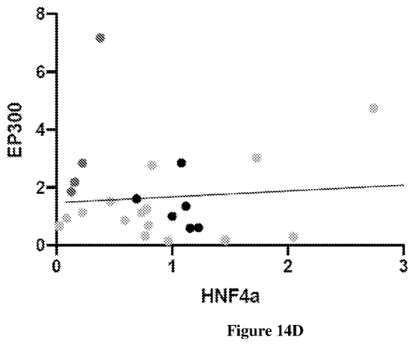

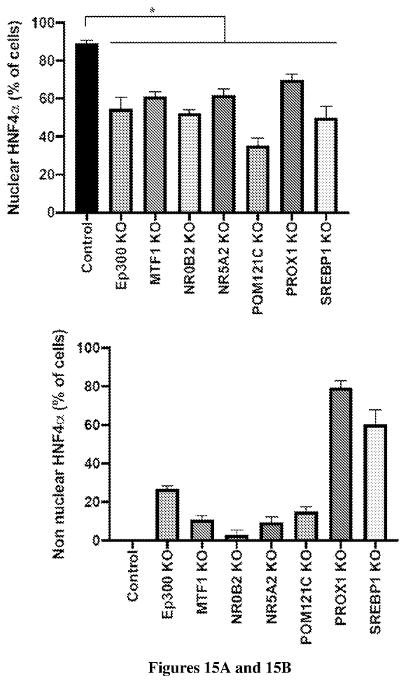

Protein expression of PROX1, NR5A2, NR0B2, MTF1, SREBP1, EP300 and POM121C correlate with extent of liver dysfunction in patients with end-stage liver failure. HNF4α must be expressed in the nucleus to function properly; therefore, the signaling pathways involved in nuclear localization of HNF4α were analyzed in hepatocytes isolated from explanted human livers with decompensated function. Because Transcriptional Factors and Regulators PROX1, NR5A2, NR0B2, MTF1, SREBP1, EP300 and POM121C were identified in the RNA-seq analysis as important modulators of HNF4α, antibody-based assays were performed for these molecules in primary human hepatocytes isolated from livers of patients undergoing liver transplantation for NASH or Alcohol-induced liver cirrhosis (Child Pugh “B” and “C”) or control normal hepatocytes. HNF4α expression was markedly reduced in decompensated liver specimens as when measured by western blot (FIG. 8A) when compared with isolated control human hepatocytes. There was also significant difference in the MTF1 expression as liver failure progressed. Moreover, using simple linear regression, Child-Pugh Scored human hepatocytes were correlated with protein expression of HNF4α and MTF1, finding that both HNF4α and MTF1 significantly correlate to the degree of liver failure (p=0.007) (FIG. 8B-8F). Additionally, it was found that protein expression of NR0B2 (FIG. 9A-D), NR5A2 (FIG. 10A-D), PROX1 (FIG. 11A-D), were significantly lower in Child Pugh C hepatocytes and their expression was correlated to the degree of hepatocyte dysfunction.

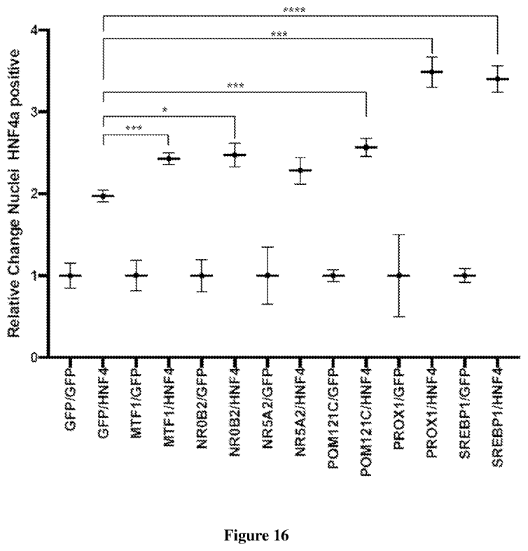

In order to understand further the role of these identified Transcriptional Factors and Regulators on HNF4α nuclear expression and location, human hepatocyte cell lines were gene edited using CRISPR/Cas9 to knockout (KO) the expression of either PROX1 or NR5A2 or NR0B2 or MTF1 or SREBP1 or EP300 and POM121C (FIG. 15A-B). It was found that by KO of PROX1, NR5A2, NR0B2, MTF1, SREBP1, EP300 or POM121C caused a significant reduction of HNF4α nuclear expression (FIG. 15A). Especially high non-nuclear expression of HNF4α was observed when PROX1 or SREBP1 was KO. Expression of HNF4α in the cytoplasm was similarly identified in previous studies on human hepatocytes with terminal liver failure. Moreover, in order to test the effect of HNF4α alone or in combination with either PROX1 or NR5A2 or NR0B2 or MTF1 or SREBP1 or POM121C to induced nuclear expression of HNF4α, treatment was performed on human hepatocytes isolated from an explanted liver of a patient with terminal liver failure due to NASH undergoing liver transplantation (FIG. 16). Ninety six hours after treatment with either HNF4α-AAV alone, about 1-fold increase was found in nuclear expression of HNF4α compared to control (GFP-AAV). However, when HNF4α -AAV treatment was combined with either PROX1-AAV or NR5A2-AAV or NR0B2-AAV or MTF1-AAV or SREBP1-AAV or POM121C-AAV, all combinations induced significantly nuclear expression of HNF4α (FIG. 16), especially when the combination involved HNF4α plus PROX1 or SREBP1.

Thus, this study demonstrates that Transcriptional Factors and Regulators PROX1, NR5A2, NR0B2, MTF1, SREBP1, EP300 and POM121C improve nuclear expression of HNF4α in cirrhotic hepatocytes with terminal liver failure. Moreover, this result shows that every combination that involves HNF4α with one or more than one Transcriptional Factors and Regulators (PROX1 or NR5A2 or NR0B2 or MTF1 or SREBP1 or EP300 and POM121C) enhances the nuclear expression of HNF4α and its reprogramming abilities to treat terminal liver failure.

### REFERENCES

Tsochatzis E A, Bosch J, Burroughs A K. Liver cirrhosis. Lancet 2014; 383:1749-1761.

Murphy S L, Xu J, Kochanek K D, Curtin S C, Arias E. Deaths: Final Data for 2015. In: Prevention CfDCa, editor.: National Vital Statistics Reports; 2017.

Goldman L, Schafer AI. Goldman-Cecil medicine. 25th edition. ed. Philadelphia, Pa.: Elsevier/Saunders, 2016: 2 volumes (xl, 2722, 12108 pages).

Lopez PM, Martin P. Update on liver transplantation: indications, organ allocation, and long-term care. Mt Sinai J Med 2006; 73:1056-1066.

Archambeaud I, Auble H, Nahon P, Planche L, Fallot G, Faroux R, Gournay J, et al. Risk factors for hepatocellular carcinoma in Caucasian patients with non-viral cirrhosis: the importance of prior obesity. Liver Int 2015; 35:1872-1876.

Donato F, Gelatti U, Limina R M, Fattovich G. Southern Europe as an example of interaction between various environmental factors: a systematic review of the epidemiologic evidence, Oncogene 2006; 25:3756-3770.

Gelatti U, Covolo L, Talamini R, Tagger A, Barbone F, Martelli C, Cremaschini F, et al. N-Acetyltransferase-2, glutathione S-transferase M1 and T1 genetic polymorphisms, cigarette smoking and hepatocellular carcinoma: a case-control study. Int J Cancer 2005; 115:301-306.

Kuper H, Tzonou A, Kaklamani E, Hsieh C C, Lagiou P, Adami H O, Trichopoulos D, et al. Tobacco smoking, alcohol consumption and their interaction in the causation of hepatocellular carcinoma. Int J Cancer 2000; 85:498-502.

Guzman-Lepe J, Cervantes-Alvarez E, Collin de l'Hortet A, Wang Y, Mars W M, Oda Y, Bekki Y, et al. Liver-enriched transcription factor expression relates to chronic hepatic failure in humans. Hepatol Commun 2018; 2:582-594.

Hernaez R, Sola E, Moreau R, Gines P. Acute-on-chronic liver failure: an update. Gut 2017; 66:541-553.

Lee Y A, Wallace MC, Friedman S L. Pathobiology of liver fibrosis: a translational success story. Gut 2015; 64:830-841.

Pessayre D, Lebrec D, Descatoire V, Peignoux M, Benhamou J P. Mechanism for reduced drug clearance in patients with cirrhosis. Gastroenterology 1978; 74:566-571.

Cichoz-Lach H, Michalak A. Oxidative stress as a crucial factor in liver diseases. World J Gastroenterol 2014; 20:8082-8091.

Simoes I C M, Fontes A, Pinton P, Zischka H, Wieckowski M R. Mitochondria in non-alcoholic fatty liver disease. Int J Biochem Cell Biol 2018; 95:93-99.

Malhi H, Kaufman R J. Endoplasmic reticulum stress in liver disease. J Hepatol 2011; 54:795-809.

Zhang X Q, Xu C F, Yu C H, Chen W X, Li Y M. Role of endoplasmic reticulum stress in the pathogenesis of nonalcoholic fatty liver disease. World J Gastroenterol 2014; 20:1768-1776.

Wang K. Molecular mechanisms of hepatic apoptosis. Cell Death Dis 2014; 5:e996.

Seki E, Schwabe R F. Hepatic inflammation and fibrosis: functional links and key pathways. Hepatology 2015; 61:1066-1079.

Zhang B H, Weltman M, Farrell G C. Does steatohepatitis impair liver regeneration? A study in a dietary model of non-alcoholic steatohepatitis in rats. J Gastroenterol Hepatol 1999; 14:133-137.

Michalopoulos G K, Khan Z. Liver Stem Cells: Experimental Findings and Implications for Human Liver Disease. Gastroenterology 2015; 149:876-882.

Dubuquoy L, Louvet A, Lassailly G, Truant S, Boleslawski E, Artru F, Maggiotto F, et al. Progenitor cell expansion and impaired hepatocyte regeneration in explanted livers from alcoholic hepatitis. Gut 2015; 64:1949-1960.

Nishikawa T, Bell A, Brooks J M, Setoyama K, Melis M, Han B, Fukumitsu K, et al. Resetting the transcription factor network reverses terminal chronic hepatic failure. J Clin Invest 2015; 125:1533-1544.

Babeu J P, Boudreau F. Hepatocyte nuclear factor 4-alpha involvement in liver and intestinal inflammatory networks. World J Gastroenterol 2014; 20:22-30.

Chellappa K, Jankova L, Schnabl J M, Pan S, Brelivet Y, Fung C L, Chan C, et al. Src tyrosine kinase phosphorylation of nuclear receptor HNF4alpha correlates with isoform-specific loss of HNF4alpha in human colon cancer. Proc Natl Acad Sci USA 2012; 109:2302-2307.

Guo H, Gao C, Mi Z, Wai P Y, Kuo P C. Phosphorylation of Ser158 regulates inflammatory redox-dependent hepatocyte nuclear factor-4a transcriptional activity. Biochem J 2014; 461:347.

Hong Y H, Varanasi U S, Yang W, Leff T. AMP-activated protein kinase regulates HNF4alpha transcriptional activity by inhibiting dimer formation and decreasing protein stability. J Biol Chem 2003; 278:27495-27501.

Lu H. Crosstalk of HNF4alpha with extracellular and intracellular signaling pathways in the regulation of hepatic metabolism of drugs and lipids. Acta Pharm Sin B 2016; 6:393-408.

Song Y, Zheng D, Zhao M, Qin Y, Wang T, Xing W, Gao L, et al. Thyroid-Stimulating Hormone Increases HNF-4alpha Phosphorylation via cAMP/PKA Pathway in the Liver. Sci Rep 2015; 5:13409.

Soutoglou E, Katrakili N, Talianidis I. Acetylation regulates transcription factor activity at multiple levels. Mol Cell 2000; 5:745-751.

Sun K, Montana V, Chellappa K, Brelivet Y, Moras D, Maeda Y, Parpura V, et al. Phosphorylation of a conserved serine in the deoxyribonucleic acid binding domain of nuclear receptors alters intracellular localization. Mol Endocrinol 2007; 21:1297-1311.

Xu Z, Tavares-Sanchez O L, Li Q, Fernando J, Rodriguez C M, Studer E J, Pandak W M, et al. Activation of bile acid biosynthesis by the p38 mitogen-activated protein kinase (MAPK): hepatocyte nuclear factor-4alpha phosphorylation by the p38 MAPK is required for cholesterol 7alpha-hydroxylase expression. J Biol Chem 2007; 282:24607-24614.

Yokoyama A, Katsura S, Ito R, Hashiba W, Sekine H, Fujiki R, Kato S. Multiple post-translational modifications in hepatocyte nuclear factor 4alpha. Biochem Biophys Res Commun 2011; 410:749-753.

Zhou W, Hannoun Z, Jaffray E, Medine C N, Black J R, Greenhough S, Zhu L, et al. SUMOylation of HNF4alpha regulates protein stability and hepatocyte function. J Cell Sci 2012; 125:3630-3635.

Gramignoli R, Green M L, Tahan V, Dorko K, Skvorak K J, Marongiu F, Zao W, et al. Development and application of purified tissue dissociation enzyme mixtures for human hepatocyte isolation. Cell Transplant 2012; 21:1245-1260.

Bell A W, Michalopoulos G K. Phenobarbital regulates nuclear expression of HNF-4alpha in mouse and rat hepatocytes independent of CAR and PXR. Hepatology 2006; 44:186-194.

Natarajan A, Wagner B, Sibilia M. The EGF receptor is required for efficient liver regeneration. Proc Natl Acad Sci USA 2007; 104:17081-17086.

Rasband W S. ImageJ, U.S. National Institutes of Health, Bethesda, Md., USA,. In.

Hainer S J, Gu W, Carone B R, Landry B D, Rando O J, Mello C C, Fazzio T G. Suppression of pervasive noncoding transcription in embryonic stem cells by esBAF. Genes Dev 2015; 29:362-378.

Kumar R, Ichihashi Y, Kimura S, Chitwood D H, Headland L R, Peng J, Maloof J N, et al. A High-Throughput Method for Illumina RNA-Seq Library Preparation. Front Plant Sci 2012; 3:202.

Morlan J D, Qu K, Sinicropi D V. Selective depletion of rRNA enables whole transcriptome profiling of archival fixed tissue. PLoS One 2012; 7:e42882.

Adiconis X, Borges-Rivera D, Satija R, DeLuca D S, Busby M A, Berlin A M, Sivachenko A, et al. Comparative analysis of RNA sequencing methods for degraded or low-input samples. Nat Methods 2013; 10:623-629.

de Hoon M J, Imoto S, Nolan J, Miyano S. Open source clustering software. Bioinformatics 2004; 20:1453-1454.

Saldanha A J. Java Treeview—extensible visualization of microarray data. Bioinformatics 2004; 20:3246-3248.

Kritis A A, Argyrokastritis A, Moschonas N K, Power S, Katrakili N, Zannis V I, Cereghini S, et al. Isolation and characterization of a third isoform of human hepatocyte nuclear factor4. Gene 1996; 173:275-280.

Tanaka T, Jiang S, Hotta H, Takano K, Iwanari H, Sumi K, Daigo K, et al. Dysregulated expression of P1 and P2 promoter-driven hepatocyte nuclear factor-4alpha in the pathogenesis of human cancer. J Pathol 2006; 208:662-672.

Walesky C, Apte U. Role of hepatocyte nuclear factor 4alpha (HNF4alpha) in cell proliferation and cancer. Gene Expr 2015; 16:101-108.

Liu L, Yannam G R, Nishikawa T, Yamamoto T, Basma H, Ito R, Nagaya M, et al. The microenvironment in hepatocyte regeneration and function in rats with advanced cirrhosis. Hepatology 2012; 55:1529-1539.

Komposch K, Sibilia M. EGFR Signaling in Liver Diseases. Int J Mol Sci 2015; 17.

Paranjpe S, Bowen W C, Mars W M, Orr A, Haynes M M, DeFrances M C, Liu S, et al. Combined systemic elimination of MET and epidermal growth factor receptor signaling completely abolishes liver regeneration and leads to liver decompensation. Hepatology 2016; 64:1711-1724.

Tsagianni A, Mars W M, Bhushan B, Bowen W C, On A, Stoops J, Paranjpe S, et al. Combined Systemic Disruption of MET and Epidermal Growth Factor Receptor Signaling Causes Liver Failure in Normal Mice. Am J Pathol 2018; 188:2223-2235.

Dekker F J, Haisma H J. Histone acetyl transferases as emerging drug targets. Drug Discov Today 2009; 14:942-948.

Argemi J, Latasa M U, Atkinson S R, Blokhin I O, Massey V, Gue J P, Cabezas J, et al. Defective HNF4alpha-dependent gene expression as a driver of hepatocellular failure in alcoholic hepatitis. Nat Commun 2019; 10:3126.

Woods A, Williams J R, Muckett P J, Mayer F V, Liljevald M, Bohlooly Y M, Carling D. Liver-Specific Activation of AMPK Prevents Steatosis on a High-Fructose Diet. Cell Rep 2017; 18:3043-3051.

Manning B D, Toker A. AKT/PKB Signaling: Navigating the Network. Cell 2017; 169:381-405.

Morales-Ruiz M, Santel A, Ribera J, Jimenez W. The Role of Akt in Chronic Liver Disease and Liver Regeneration. Semin Liver Dis 2017; 37:11-16.

Alam A, Chun Suen K, Ma D. Acute-on-chronic liver failure: recent update. J Biomed Res 2017; 31:283-300.

B. Sosa-Pineda, J. T. Wigle, G. Oliver Hepatocyte migration during liver development requires Proxl Nat. Genet., 25 (2000), pp. 254-255.

Song K H, Li T and Chiang J Y (2006) A prospero-related homeodomain protein is a novel co-regulator of hepatocyte nuclear factor 4alpha that regulates the cholesterol 7alpha-hydroxylase gene. J Biol Chem 281, 10081-10088.

Xu S Z. Proxl Facilitates Transfected CHO Cell Proliferation through Activation of the AKT Signaling Pathway. Int J Biomed Sci. 2010 Mar. 6(1):49-59.

Rausa F M, Galarneau L, Belanger L, Costa R H. 1999. The nuclear receptor fetoprotein transcription factor is coexpressed with its target HNF-3b in the developing murine liver intestine and pancreas. Mech Dev 89: 185-188.

Labelle-Dumais C, Jacob-Wagner M, Paré J F, Bélanger L, Dufort D. Nuclear receptor NR5A2 is required for proper primitive streak morphogenesis. Dev Dyn. 2006 December 235(12):3359-69.

Kim D H, Kwon S, Byun S, Xiao Z, Park S, Wu S Y, Chiang C M, Kemper B, Kemper J K. Critical role of RanBP2-mediated SUMOylation of Small Heterodimer Partner in maintaining bile acid homeostasis. Nat Commun. 2016 Jul. 14 7:12179.

Lindert U, Cramer M, Meuli M, Georgiev O, Schaffner W. Metal-responsive transcription factor 1 (MTF-1) activity is regulated by a nonconventional nuclear localization signal and a metal-responsive transactivation domain Mol Cell Biol. 2009 Dec. 29(23):6283-93. doi: 10.1128/MCB.00847-09.

Rutherford J C, Bird A J. Metal-responsive transcription factors that regulate iron, zinc, and copper homeostasis in eukaryotic cells. Eukaryot Cell. 2004 Feb. 3(1):1-13.

Shi Q, Hoffman B, Liu Q, PI3K-Akt signaling pathway upregulates hepatitis C virus RNA translation through the activation of SREBPs. Virology. 2016 Mar. 4900:99-108.

Porstmann T, Santos C R, Griffiths B, Cully M, Wu M, Leevers S, Griffiths J R, Chung Y L, Schulze A. SREBP activity is regulated by mTORC1 and contributes to Akt-dependent cell growth. Cell Metab. 2008 Sep. 8(3):224-36.

Breaux M, Lewis K, Valanejad L, Iakova P, Chen F, Mo Q, Medrano E, Timchenko L, Timchenko N. p300 Regulates Liver Functions by Controlling p53 and C/EBP Family Proteins through Multiple Signaling Pathways. Mol Cell Biol. 2015 Sep. 1; 35(17):3005-16.

He L, Cao J, Meng S, Ma A, Radovick S, Wondisford F E, Activation of basal gluconeogenesis by coactivator p300 maintains hepatic glycogen storage. Mol Endocrinol. 2013 Aug. 27(8):1322-32.

Stavru F, Nautrup-Pedersen G, Cordes V C, Gorlich D. Nuclear pore complex assembly and maintenance in POM121- and gp210-deficient cells. J Cell Biol. 2006 May 22; 173(4):477-83.

Fusakio M E, Willy J A, Wang Y, Mirek E T, Al Baghdadi R J, Adams C M, Anthony T G, Wek R C. Transcription factor ATF4 directs basal and stress-induced gene expression in the unfolded protein response and cholesterol metabolism in the liver. Mol Biol Cell. 2016 May 1; 27(9):1536-51.

Malhi H, Kaufman R J. Endoplasmic reticulum stress in liver disease. J Hepatol. 2011 Apr. 54(4):795-809.

Florentino R M, Fraunhoffer N A, Morita K, Takeishi K, Ostrowska A, Achreja A, Animasahun O, Haep N, Arazov S, Agarwal N, Collin de l'Hortet A, Guzman-Lepe J, Tafaleng E N, Mukherjee A, Troy K, Banerjee S, Paranjpe S, Michalopoulos G K, Bell A, Nagrath D, Hainer S J, Fox I J, Soto-Gutierrez A. Cellular Location of HNF4α is Linked With Terminal Liver Failure in Humans. Hepatol Commun. 2020 Apr. 21; 4(6):859-875.

Guzman-Lepe J, Cervantes-Alvarez E, Collin de l'Hortet A, Wang Y, Mars W M, Oda Y, Bekki Y, Shimokawa M, Wang H, Yoshizumi T, Maehara Y, Bell A, Fox I J, Takeishi K, Soto-Gutierrez A. Hepatol Commun. Liver-enriched transcription factor expression relates to chronic hepatic failure in humans. 2018 Mar. 23; 2(5):582-594.

Nishikawa T, Bellance N, Damm A, Bing H, Zhu Z, Handa K, Yovchev M I, Sehgal V, Moss T J, Oertel M, Ram P T, Pipinos II, Soto-Gutierrez A, Fox I J, Nagrath D. A switch in the source of ATP production and a loss in capacity to perform glycolysis are hallmarks of hepatocyte failure in advance liver disease. J Hepatol. 2014 Jun.; 60(6):1203-11.

Nishikawa T, Bell A, Brooks J M, Setoyama K, Melis M, Han B, Fukumitsu K, Handa K, Tian J, Kaestner K H, Vodovotz Y, Locker J, Soto-Gutierrez A, Fox I J. Resetting the transcription factor network reverses terminal chronic hepatic failure. J Clin Invest. 2015 Apr.; 125(4):1533-44.

1. A method of treating a liver disease in a subject in need thereof.comprising administering to the subject a composition, wherein the composition increases an amount or function of one or more transcription factors selected from the group consisting of PROX1, NR5A2, NR0B2, MTF1, SREBP1, EP300, and POM121C.

2. The method of claim 1, wherein the composition is a vector, and wherein the vector comprises one or more nucleic acids that encode the one or more of PROX1, NR5A2, NR0B2, MTF1, SREBP1, EP300, and POM121C.

3. The method of claim 1, wherein the composition is a vector, and wherein the vector comprises one or more nucleic acids that encode the PROX1 and/or SREBP1.

4. The method of claim 1, wherein the one or more nucleic acids are a DNA or a mRNA.

5. The method of claim 1, wherein the administration of the composition increases an amount of HNF4α in a nucleus of a hepatocyte in the subject.

6. The method of claim 1, wherein the administration of the composition does not increase a total amount of HNF4α in the hepatocyte.

7. The method of claim 1, wherein the administration of the composition increases a total amount of HNF4α in the hepatocyte.

8. The method of claim 1, wherein the vector further comprises a nucleic acid that encodes HNF4α.

9. The method of claim 1, further comprising administering to the subject a vector that comprises a nucleic acid that encodes HNF4α.

10. The method of claim 8 , wherein the nucleic acid encodes HNF4α isoform 2 (P1).

11. The method of claim 8, wherein the nucleic acid that encodes HNF4α comprises SEQ ID NO: 1.

12. The method of claim 1, wherein the liver disease is liver fibrosis, liver cirrhosis, liver cancer, or end-stage liver disease.

13. The method of claim 1, wherein the liver disease is liver cirrhosis.

14. The method of claim 1, wherein the subject is a human.

15. A method of treating a liver disease in a subject in need thereof comprising administering to the subject a composition, wherein the composition decreases an amount or suppresses a function of one or more transcription factors selected from the group consisting of DNAJB1/HSP40, ATF6, ATF4, and PERK.

16. The method of claim 15, wherein the composition is a nucleic acid.

17. The method of claim 16, wherein the nucleic acid is a DNA or a RNA.

18. The method of claim 15, wherein the administration of the nucleic acid increases an amount of HNF4α in a nucleus of a hepatocyte in the subject.

19. The method of claim 15, wherein the administration of the composition does not increase a total amount of HNF4α in the hepatocyte.

20. The method of claim 15, wherein the administration of the composition increases a total amount of HNF4α in the hepatocyte.

21. The method of claim 15, wherein the composition further comprises a nucleic acid that encodes HNF4α.

22. The method of claim 15, further comprising administering to the subject a vector that comprises a nucleic acid that encodes HNF4α.

23. The method of claim 21, wherein the nucleic acid encodes HNF4α isoform 2.

24. The method of claim 21, wherein the nucleic acid that encodes HNF4α comprises SEQ ID NO: 1.

25. The method of claim 15, wherein the liver disease comprises liver fibrosis, liver cirrhosis, liver cancer, or an end-stage liver disease.

26. The method of claim 15, wherein the subject is a human.

27. A composition comprising a vector, wherein the vector comprises one or more nucleic acids that encode one or more transcription factors selected from the group consisting of PROX1, NR5A2, NR0B2, MTF1, SREBP1, EP300, and POM121C, and functional fragments thereof.

28. The composition of claim 27, further comprising another vector comprising a nucleic acid that encodes HNF4α.

29. The composition of claim 28, wherein the nucleic acid that encodes HNF4α comprises SEQ ID NO:1.

30. A method of treating a liver disease in a subject in need thereof comprising administering to the subject a vector that comprises nucleic acid encodes HNF4α isoform 2.

31. The method of claim 30, wherein the nucleic acid comprises SEQ ID NO:1.

32. The method of claim 30, wherein the liver disease is liver fibrosis, liver cirrhosis, liver cancer, or an end-stage liver disease.

33. The method of claim 32, wherein the liver disease is liver cirrhosis.

34. The method of claim 30, wherein the subject is a human.


SEQUENCE LISTING (34 sequences)

SEQ ID NO: 1 - protein, 474 aa, Homo sapiens
MRLSKTLVDMDMADYSAALDPAYTTLEFENVQVLTMGNDTSPSEGTNLNAPNSLGVSALCAICGDRATGKHYGASSCDGCKGFFRRSVRKNHMYSCRFSRQCVVDKDKRNQCRYCRLKKCFRAGMKKEAVQNERDRISTRRSSYEDSSLPSINALLQAEVLSRQITSPVSGINGDIRAKKIASIADVCESMKEQLLVLVEWAKYIPAFCELPLDDQVALLRAHAGEHLLLGATKRSMVFKDVLLLGNDYIVPRHCPELAEMSRVSIRILDELVLPFQELQIDDNEYAYLKAIIFFDPDAKGLSDPGKIKRLRSQVQVSLEDYINDRQYDSRGRFGELLLLLPTLQSITWQMIEQIQFIKLFGMAKIDNLLQEMLLGGSPSDAPHAHHPLHPHLMQEHMGTNVIVANTMPTHLSNGQMCEWPRPRGQAATPETPQPSPPGGSGSEPYKLLPGAVATIVKPLSAIPQPTITKQEVI

SEQ ID NO: 2 - protein, 737 aa, Homo sapiens
MPDHDSTALLSRQTKRRRVDIGVKRTVGTASAFFAKARATFFSAMNPQGSEQDVEYSVVQHADGEKSNVLRKLLKRANSYEDAMMPFPGATIISQLLKNNMNKNGGTEPSFQASGLSSTGSEVHQEDICSNSSRDSPPECLSPFGRPTMSQFDMDRLCDEHLRAKRARVENIIRGMSHSPSVALRGNENEREMAPQSVSPRESYRENKRKQKLPQQQQQSFQQLVSARKEQKREERRQLKQQLEDMQKQLRQLQEKFYQIYDSTDSENDEDGNLSEDSMRSEILDARAQDSVGRSDNEMCELDPGQFIDRARALIREQEMAENKPKREGNNKERDHGPNSLQPEGKHLAETLKQELNTAMSQVVDTVVKVFSAKPSRQVPQVFPPLQIPQARFAVNGENHNFHTANQRLQCFGDVIIPNPLDTFGNVQMASSTDQTEALPLVVRKNSSDQSASGPAAGGHHQPLHQSPLSATTGFTTSTFRHPFPLPLMAYPFQSPLGAPSGSFSGKDRASPESLDLTRDTTSLRTKMSSHHLSHHPCSPAHPPSTAEGLSLSLIKSECGDLQDMSEISPYSGSAMQEGLSPNHLKKAKLMFFYTRYPSSNMLKTYFSDVKFNRCITSQLIKWFSNFREFYYIQMEKYARQAINDGVTSTEELSITRDCELYRALNMHYNKANDFEVPERFLEVAQITLREFFNAIIAGKDVDPSWKKAIYKVICKLDSEVPEIFKSPNCLQELLHE

SEQ ID NO: 3 - protein, 541 aa, Homo sapiens
MSSNSDTGDLQESLKHGLTPIGAGLPDRHGSPIPARGRLVMLPKVETEALGLARSHGEQGQMPENMQVSQFKMVNYSYDEDLEELCPVCGDKVSGYHYGLLTCESCKGFFKRTVQNNKRYTCIENQNCQIDKTQRKRCPYCRFQKCLSVGMKLEAVRADRMRGGRNKFGPMYKRDRALKQQKKALIRANGLKLEAMSQVIQAMPSDLTISSAIQNIHSASKGLPLNHAALPPTDYDRSPFVTSPISMTMPPHGSLQGYQTYGHFPSRAIKSEYPDPYTSSPESIMGYSYMDSYQTSSPASIPHLILELLKCEPDEPQVQAKIMAYLQQEQANRSKHEKLSTFGLMCKMADQTLFSIVEWARSSIFFRELKVDDQMKLLQNCWSELLILDHIYRQVVHGKEGSIFLVTGQQVDYSIIASQAGATLNNLMSHAQELVAKLRSLQFDQREFVCLKFLVLFSLDVKNLENFQLVEGVQEQVNAALLDYTMCNYPQQTEKFGQLLLRLPEIRAISMQAEEYLYYKHLNGDVPYNNLLIEMLHAKRA

SEQ ID NO: 4 - protein, 257 aa, Homo sapiens
MSTSQPGACPCQGAASRPAILYALLSSSLKAVPRPRSRCLCRQHRPVQLCAPHRTCREALDVLAKTVAFLRNLPSFWQLPPQDQRRLLQGCWGPLFLLGLAQDAVTFEVAEAPVPSILKKILLEEPSSSGGSGQLPDRPQPSLAAVQWLQCCLESFWSLELSPKEYACLKGTILFNPDVPGLQAASHIGHLQQEAHWVLCEVLEPWCPAAQGRLTRVLLTASTLKSIPTSLLGDLFFRPIIGDVDIAGLLGDMLLLR

SEQ ID NO: 5 - protein, 753 aa, Homo sapiens
MGEHSPDNNIIYFEAEEDELTPDDKMLRFVDKNGLVPSSSGTVYDRTTVLIEQDPGTLEDEDDDGQCGEHLPFLVGGEEGFHLIDHEAMSQGYVQHIISPDQIHLTINPGSTPMPRNIEGATLTLQSECPETKRKEVKRYQCTFEGCPRTYSTAGNLRTHQKTHRGEYTFVCNQEGCGKAFLTSYSLRIHVRVHTKEKPFECDVQGCEKAFNTLYRLKAHQRLHTGKTFNCESEGCSKYFTTLSDLRKHIRTHTGEKPFRCDHDGCGKAFAASHHLKTHVRTHTGERPFFCPSNGCEKTFSTQYSLKSHMKGHDNKGHSYNALPQHNGSEDTNHSLCLSDLSLLSTDSELRENSSTTQGQDLSTISPAIIFESMFQNSDDTAIQEDPQQTASLTESFNGDAESVSDVPPSTGNSASLSLPLVLQPGLSEPPQPLLPASAPSAPPPAPSLGPGSQQAAFGNPPALLQPPEVPVPHSTQFAANHQEFLPHPQAPQPIVPGLSVVAGASASAAAVASAVAAPAPPQSTTEPLPAMVQTLPLGANSVLTNNPTITITPTPNTAILQSSLVMGEQNLQWILNGATSSPQNQEQIQQASKVEKVFFTTAVPVASSPGSSVQQIGLSVPVIIIKQEEACQCQCACRDSAKERASSRRKGCSSPPPPEPSPQAPDGPSLQLPAQTFSSAPVPGSSSSTLPSSCEQSRQAETPSDPQTETLSAMDVSEFLSLQSLDTPSNLIPIEALLQGEEEMGLTSSFSK

SEQ ID NO: 6 - protein, 1147 aa, Homo sapiens
MDEPPFSEAALEQALGEPCDLDAALLTDIEDMLQLINNQDSDFPGLFDPPYAGSGAGGTDPASPDTSSPGSLSPPPATLSSSLEAFLSGPQAAPSPLSPPQPAPTPLKMYPSMPAFSPGPGIKEESVPLSILQTPTPQPLPGALLPQSFPAPAPPQFSSTPVLGYPSPPGGFSTGSPPGNTQQPLPGLPLASPPGVPPVSLHTQVQSVVPQQLLTVTAAPTAAPVTTTVTSQIQQVPVLLQPHFIKADSLLLTAMKTDGATVKAAGLSPLVSGTTVQTGPLPTLVSGGTILATVPLVVDAEKLPINRLAAGSKAPASAQSRGEKRTAHNAIEKRYRSSINDKIIELKDLVVGTEAKLNKSAVLRKAIDYIRFLQHSNQKLKQENLSLRTAVHKSKSLKDLVSACGSGGNTDVLMEGVKTEVEDTLTPPPSDAGSPFQSSPLSLGSRGSGSGGSGSDSEPDSPVFEDSKAKPEQRPSLHSRGMLDRSRLALCTLVFLCLSCNPLASLLGARGLPSPSDTTSVYHSPGRNVLGTESRDGPGWAQWLLPPVVWLLNGLLVLVSLVLLFVYGEPVTRPHSGPAVYFWRHRKQADLDLARGDFAQAAQQLWLALRALGRPLPTSHLDLACSLLWNLIRHLLQRLWVGRWLAGRAGGLQQDCALRVDASASARDAALVYHKLHQLHTMGKHTGGHLTATNLALSALNLAECAGDAVSVATLAEIYVAAALRVKTSLPRALHFLTRFFLSSARQACLAQSGSVPPAMQWLCHPVGHRFFVDGDWSVLSTPWESLYSLAGNPVDPLAQVTQLFREHLLERALNCVTQPNPSPGSADGDKEFSDALGYLQLLNSCSDAAGAPAYSFSISSSMATTTGVDPVAKWWASLTAVVIHWLRRDEEAAERLCPLVEHLPRVLQESERPLPRAALHSFKAARALLGCAKAESGPASLTICEKASGYLQDSLATTPASSSIDKAVQLFLCDLLLVVRTSLWRQQQPPAPAPAAQGTSSRPQASALELRGFQRDLSSLRRLAQSFRPAMRRVFLHEATARLMAGASPTRTHQLLDRSLRRRAGPGGKGGAVAELEPRPTRREHAEALLLASCYLPPGFLSAPGQRVGMLAEAARTLEKLGDRRLLHDCQQMLMRLGGGTTVTSS

SEQ ID NO: 7 - protein, 2414 aa, Homo sapiens
MAENVVEPGPPSAKRPKLSSPALSASASDGTDFGSLFDLEHDLPDELINSTELGLTNGGDINQLQTSLGMVQDAASKHKQLSELLRSGSSPNLNMGVGGPGQVMASQAQQSSPGLGLINSMVKSPMTQAGLTSPNMGMGTSGPNQGPTQSTGMMNSPVNQPAMGMNTGMNAGMNPGMLAAGNGQGIMPNQVMNGSIGAGRGRQNMQYPNPGMGSAGNLLTEPLQQGSPQMGGQTGLRGPQPLKMGMMNNPNPYGSPYTQNPGQQIGASGLGLQIQTKTVLSNNLSPFAMDKKAVPGGGMPNMGQQPAPQVQQPGLVTPVAQGMGSGAHTADPEKRKLIQQQLVLLLHAHKCQRREQANGEVRQCNLPHCRTMKNVLNHMTHCQSGKSCQVAHCASSRQIISHWKNCTRHDCPVCLPLKNAGDKRNQQPILTGAPVGLGNPSSLGVGQQSAPNLSTVSQIDPSSIERAYAALGLPYQVNQMPTQPQVQAKNQQNQQPGQSPQGMRPMSNMSASPMGVNGGVGVQTPSLLSDSMLHSAINSQNPMMSENASVPSLGPMPTAAQPSTTGIRKQWHEDITQDLRNHLVHKLVQAIFPTPDPAALKDRRMENLVAYARKVEGDMYESANNRAEYYHLLAEKIYKIQKELEEKRRTRLQKQNMLPNAAGMVPVSMNPGPNMGQPQPGMTSNGPLPDPSMIRGSVPNQMMPRITPQSGLNQFGQMSMAQPPIVPRQTPPLQHHGQLAQPGALNPPMGYGPRMQQPSNQGQFLPQTQFPSQGMNVTNIPLAPSSGQAPVSQAQMSSSSCPVNSPIMPPGSQGSHIHCPQLPQPALHQNSPSPVPSRTPTPHHTPPSIGAQQPPATTIPAPVPTPPAMPPGPQSQALHPPPRQTPTPPTTQLPQQVQPSLPAAPSADQPQQQPRSQQSTAASVPTPTAPLLPPQPATPLSQPAVSIEGQVSNPPSTSSTEVNSQAIAEKQPSQEVKMEAKMEVDQPEPADTQPEDISESKVEDCKMESTETEERSTELKTEIKEEEDQPSTSATQSSPAPGQSKKKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDRFVYTCNECKHHVETRWHCTVCEDYDLCITCYNTKNHDHKMEKLGLGLDDESNNQQAAATQSPGDSRRLSIQRCIQSLVHACQCRNANCSLPSCQKMKRVVQHTKGCKRKTNGGCPICKQLIALCCYHAKHCQENKCPVPFCLNIKQKLRQQQLQHRLQQAQMLRRRMASMQRTGVVGQQQGLPSPTPATPTTPTGQQPTTPQTPQPTSQPQPTPPNSMPPYLPRTQAAGPVSQGKAAGQVTPPTPPQTAQPPLPGPPPAAVEMAMQIQRAAETQRQMAHVQIFQRPIQHQMPPMTPMAPMGMNPPPMTRGPSGHLEPGMGPTGMQQQPPWSQGGLPQPQQLQSGMPRPAMMSVAQHGQPLNMAPQPGLGQVGISPLKPGTVSQQALQNLLRTLRSPSSPLQQQQVLSILHANPQLLAAFIKQRAAKYANSNPQPIPGQPGMPQGQPGLQPPTMPGQQGVHSNPAMQNMNPMQAGVQRAGLPQQQPQQQLQPPMGGMSPQAQQMNMNHNTMPSQFRDILRRQQMMQQQQQQGAGPGIGPGMANHNQFQQPQGVGYPPQQQQRMQHHMQQMQQGNMGQIGQLPQALGAEAGASLQAYQQRLLQQQMGSPVQPNPMSPQQHMLPNQAQSPHLQGQQIPNSLSNQVRSPQPVPSPRPQSQPPHSSPSPRMQPQPSPHHVSPQTSSPHPGLVAAQANPMEQGHFASPDQNSMLSQLASNPGMANLHGASATDLGLSTDNSDLNSNLSQSTLDIH

SEQ ID NO: 8 - protein, 1229 aa, Homo sapiens
MSPAAAAAGAGERRRPIASVRDGRGRGCGGPAGAALLGLSLVGLLLYLVPAAAALAWLAVGTTAAWWGLSREPRGSRPLSSFVQKARHRRTLFASPPAKSTANGNLLEPRTLLEGPDPAELLLMGSYLGKPGPPQPAPAPEGQDLRNRPGRRPPARPAPRSTPPSQPTHRVHHFYPSLPTPLLRPSGRPSPRDRGTLPDRFVITPRRRYPIHQTQYSCPGVLPTVCWNGYHKKAVLSPRNSRMVCSPVTVRIAPPDRRFSRSAIPEQIISSTLSSPSSNAPDPCAKETVLSALKEKKKKRTVEEEDQIFLDGQENKRRRHDSSGSGHSAFEPLVASGVPASFVPKPGSLKRGLNSQSSDDHLNKRSRSSSMSSLTGAYTSGIPSSSRNAITSSYSSTRGISQLWKRNGPSSSPFSSPASSRSQTPERPAKKIREEELCHHSSSSTPLAADKESQGEKAADTTPRKKQNSNSQSTPGSSGQRKRKVQLLPSRRGEQLTLPPPPQLGYSITAEDLDLEKKASLQWFNQALEDKSDAASNSVTETPPTTQPSFTFTLPAAATASPPTSLLAPSTNPLLESLKKMQTPPSLPPCPESAGAATTEALSPPKTPSLLPPLGLSQSGPPGLLPSPSFDSKPPTTLLGLIPAPSMVPATDTKAPPTLQAETATKPQATSAPSPAPKQSFLFGTQNTSPSSPAAPAASSASPMFKPIFTAPPKSEKEGLTPPGPSVSATAPSSSSLPTTTSTTAPTFQPVFSSMGPPASVPLPAPFFKQTTTPATAPTTTAPLFTGLASATSAVAPITSASPSTDSASKPAFGFGINSVSSSSVSTTTSTATAASQPFLFGAPQASAASFTPAMGSIFQFGKPPALPTTTTVTTFSQSLPTAVPTATSSSAADFSGFGSTLATSAPATSSQPTLTFSNTSTPTFNIPFGSSAKSPLPSYPGANPQPAFGAAEGQPPGAAKPALTPSFGSSFTFGNSAAPAPATAPTPAPASTIKIVPAHVPTPIQPTFGGATHSAFGLKATASAFGAPASSQPAFGGSTAVFSFGAATSSGFGATTQTASSGSSSSVFGSTTPSPFTFGGSAAPAGSGSFGINVATPGSSATTGAFSFGAGQSGSTATSTPFTGGLGQNALGTTGQSTPFAFNVGSTTESKPVFGGTATPTFGQNTPAPGVGTSGSSLSFGASSAPAQGFVGVGPFGSAAPSFSIGAGSKTPGARQRLQARRQHTRKK

SEQ ID NO: 9 - protein, 339 aa, Homo sapiens
GKDYYQTLGLARGASDEEIKRAYRRQALRYHPDKNKEPGAEEKFKEIAEAYDVLSDPRKREIFDRYGEEGLKGSGPSGGSGGGANGTSFSYTFHGDPHAMFAEFFGGRNPFDTFFGQRNGEEGMDIDDPFSGFPMGMGGFTNVNFGRSRSAQEPARKKQDPPVTHDLRVSLEEIYSGCTKKMKISHKRLNPDGKSIRNEDKILTIEVKKGWKEGTKITFPKEGDQTSNNIPADIVFVLKDKPHNIFKRDGSDVIYPARISLREALCGCTVNVPTLDGRTIPVVFKDVIRPGMRRKVPGEGLPLPKTPEKRGDLIIEFEVIFPERIPQTSRTVLEQVLPI

SEQ ID NO: 10 - protein, 670 aa, Homo sapiens
MGEPAGVAGTMESPFSPGLFHRLDEDWDSALFAELGYFTDTDELQLEAANETYENNFDNLDFDLDLMPWESDIWDINNQICTVKDIKAEPQPLSPASSSYSVSSPRSVDSYSSTQHVPEELDLSSSSQMSPLSLYGENSNSLSSAEPLKEDKPVTGPRNKTENGLTPKKKIQVNSKPSIQPKPLLLPAAPKTQTNSSVPAKTIIIQTVPTLMPLAKQQPIISLQPAPTKGQTVLLSQPTVVQLQAPGVLPSAQPVLAVAGGVTQLPNHVVNVVPAPSANSPVNGKLSVTKPVLQSTMRNVGSDIAVLRRQQRMIKNRESACQSRKKKKEYMLGLEARLKAALSENEQLKKENGTLKRQLDEVVSENQRLKVPSPKRRVVCVMIVLAFIILNYGPMSMLEQDSRRMNPSVSPANQRRHLLGFSAKEAQDTSDGIIQKNSYRYDHSVSNDKALMVLTEEPLLYIPPPPCQPLINTTESLRLNHELRGWVHRHEVERTKSRRMTNNQQKTRILQGALEQGSNSQLMAVQYTETTSSISRNSGSELQVYYASPRSYQDFFEAIRRRGDTFYVVSFRRDHLLLPATTHNKTTRPKMSIVLPAININENVINGQDYEVMMQIDCQVMDTRILHIKSSSVPPYLRDQQRNQTNTFFGSPPAATEATHVVSTIPESLQ

SEQ ID NO: 11 - protein, 351 aa, Homo sapiens
MTEMSFLSSEVLVGDLMSPFDQSGLGAEESLGLLDDYLEVAKHFKPHGFSSDKAKAGSSEWLAVDGLVSPSNNSKEDAFSGTDWMLEKMDLKEFDLDALLGIDDLETMPDDLLTTLDDTCDLFAPLVQETNKQPPQTVNPIGHLPESLTKPDQVAPFTFLQPLPLSPGVLSSTPDHSFSLELGSEVDITEGDRKPDYTAYVAMIPQCIKEEDTPSDNDSGICMSPESYLGSPQHSPSTRGSPNRSLPSPGVLCGSARPKPYDPPGEKMVAAKVKGEKLDKKLKKMEQNKTAATRYRQKKRAEQEALTGECKELEKKNEALKERADSLAKEIQYLKDLIEEVRKARGKKRVP

SEQ ID NO: 12 - protein, 1116 aa, Homo sapiens
MERAISPGLLVRALLLLLLLLGLAARTVAAGRARGLPAPTAEAAFGLGAAAAPTSATRVPAAGAVAAAEVTVEDAEALPAAAGEQEPRGPEPDDETELRPRGRSLVIISTLDGRIAALDPENHGKKQWDLDVGSGSLVSSSLSKPEVFGNKMIIPSLDGALFQWDQDRESMETVPFTVESLLESSYKFGDDVVLVGGKSLTTYGLSAYSGKVRYICSALGCRQWDSDEMEQEEDILLLQRTQKTVRAVGPRSGNEKWNFSVGHFELRYIPDMETRAGFIESTFKPNENTEESKIISDVEEQEAAIMDIVIKVSVADWKVMAFSKKGGHLEWEYQFCTPIASAWLLKDGKVIPISLFDDTSYTSNDDVLEDEEDIVEAARGATENSVYLGMYRGQLYLQSSVRISEKFPSSPKALESVTNENAIIPLPTIKWKPLIHSPSRTPVLVGSDEFDKCLSNDKFSHEEYSNGALSILQYPYDNGYYLPYYKRERNKRSTQITVRFLDNPHYNKNIRKKDPVLLLHWWKEIVATILFCIIATTFIVRRLFHPHPHRQRKESETQCQTENKYDSVSGEANDSSWNDIKNSGYISRYLTDFEPIQCLGRGGFGVVFEAKNKVDDCNYAIKRIRLPNRELAREKVMREVKALAKLEHPGIVRYFNAWLEAPPEKWQEKMDEIWLKDESTDWPLSSPSPMDAPSVKIRRMDPFATKEHIEIIAPSPQRSRSFSVGISCDQTSSSESQFSPLEFSGMDHEDISESVDAAYNLQDSCLTDCDVEDGTMDGNDEGHSFELCPSEASPYVRSRERTSSSIVFEDSGCDNASSKEEPKTNRLHIGNHCANKLTAFKPTSSKSSSEATLSISPPRPTTLSLDLTKNTTEKLQPSSPKVYLYIQMQLCRKENLKDWMNGRCTIEERERSVCLHIFLQIAEAVEFLHSKGLMHRDLKPSNIFFTMDDVVKVGDFGLVTAMDQDEEEQTVLTPMPAYARHTGQVGTKLYMSPEQIHGNSYSHKVDIFSLGLILFELLYPFSTQMERVRTLTDVRNLKFPPLFTQKYPCEYVMVQDMLSPSPMERPEAINIIENAVFEDLDFPGKTVLRQRSRSLSSSGTKHSRQSNNSHSPLPSN

SEQ ID NO: 13 - DNA, 8468 bp, Homo sapiens
agctgagggagcgctctgaaataatacaccattgcagccggggaaagcagagcggcgcaaaagagctctcgccgggtccgcctgctccctctccgcttcgctcctcttctcttctttacccttctcctctctcctcctctgctgctctctcctctcctcccgctcttctctctcctcctctcctgctctctcctcttcccttagctcctcttcttttcttctcctcttcttccctctcctcgcctctcccctgctcctcttctctcgtctcccctcccctcccgcctctctctcccctctccctctcccactcgccccgctcgctcgctcgctgtcgcacagactcaccgtcccttgtccaattatcatattcatcacccgcaagatatcaccgtgtgtgcactcgcgtgttttcctctctctgccgggggaaaaaaaagagagagagagagatagagagagagagagagagagagagagagaggctcggtcccactgctccctgcaccgcggtcccgggattcttgagctgtgcccagctgacgagcttttgaagatggcacaataaccgtccagtgatgcctgaccatgacagcacagccctcttaagccggcaaaccaagaggagaagagttgacattggagtgaaaaggacggtagggacagcatctgcattttttgctaaggcaagagcaacgttttttagtgccatgaatccccaaggttctgagcaggatgttgagtattcagtggtgcagcatgcagatggggaaaagtcaaatgtactccgcaagctgctgaagagggcgaactcgtatgaagatgccatgatgccttttccaggagcaaccataatttcccagctgttgaaaaataacatgaacaaaaatggtggcacggagcccagtttccaagccagcggtctctctagtacaggctccgaagtacatcaggaggatatatgcagcaactcttcaagagacagccccccagagtgtctttccccttttggcaggcctactatgagccagtttgatatggatcgcttatgtgatgagcacctgagagcaaagcgcgcccgggttgagaatataattcggggtatgagccattcccccagtgtggcattaaggggcaatgaaaatgaaagagagatggccccgcagtctgtgagtccccgagaaagttacagagaaaacaaacgcaagcaaaagcttccccagcagcagcaacagagtttccagcagctggtttcagcccgaaaagaacagaagcgagaggagcgccgacagctgaaacagcagctggaggacatgcagaaacagctgcgccagctgcaggaaaagttctaccaaatctatgacagcactgattcggaaaatgatgaagatggtaacctgtctgaagacagcatgcgctcggagatcctggatgccagggcccaggactctgtcggaaggtcagataatgagatgtgcgagctagacccaggacagtttattgaccgagctcgagccctgatcagagagcaggaaatggctgaaaacaagccgaagcgagaaggcaacaacaaagaaagagaccatgggccaaactccttacaaccggaaggcaaacatttggctgagaccttgaaacaggaactgaacactgccatgtcgcaagttgtggacactgtggtcaaagtcttttcggccaagccctcccgccaggttcctcaggtcttcccacctctccagatcccccaggccagatttgcagtcaatggggaaaaccacaatttccacaccgccaaccagcgcctgcagtgctttggcgacgtcatcattccgaaccccctggacacctttggcaatgtgcagatggccagttccactgaccagacagaagcactgcccctggttgtccgcaaaaactcctctgaccagtctgcctccggccctgccgctggcggccaccaccagcccctgcaccagtcgcctctctctgccaccacgggcttcaccacgtccaccttccgccaccccttcccccttcccttgatggcctatccatttcagagcccattaggtgctccctccggctccttctctggaaaagacagagcctctcctgaatccttagacttaactagggataccacgagtctgaggaccaagatgtcatctcaccacctgagccaccacccttgttcaccagcacacccgcccagcaccgccgaagggctctccttgtcgctcataaagtccgagtgcggcgatcttcaagatatgtctgaaatatcaccttattcgggaagtgcaatgcaggaaggattgtcacccaatcacttgaaaaaagcaaagctcatgtttttttatacccgttatcccagctccaatatgctgaagacctacttctccgacgtaaagttcaacagatgcattacctctcagctcatcaagtggtttagcaatttccgtgagttttactacattcagatggagaagtacgcacgtcaagccatcaacgatggggtcaccagtactgaagagctgtctataaccagagactgtgagctgtacagggctctgaacatgcactacaataaagcaaatgactttgaggttccagagagattcctggaagttgctcagatcacattacgggagtttttcaatgccattatcgcaggcaaagatgttgatccttcctggaagaaggccatatacaaggtcatctgcaagctggatagtgaagtccctgagattttcaaatccccgaactgcctacaagagctgcttcatgagtagaaatttcaacaactctttttgaatgtatgaagagtagcagtcccctttggatgtccaagttatatgtgtctagattttgatttcatatatatgtgtatgggaggcatggatatgttatgaaatcagctggtaattcctcctcatcacgtttctctcattttcttttgttttccattgcaaggggatggttgttttctttctgcctttagtttgcttttgcccaaggcccttaacatttggacacttaaaatagggttaattttcagggaaaaagaatgttggcgtgtgtaaagtctctattagcaatgaagggaatttgttaacgatgcatccacttgattgatgacttattgcaaatggcggttggctgaggaaaacccatgacacagcacaactctacagacagtgatgtgtctcttgtttctactgctaagaaggtctgaaaatttaatgaaaccacttcatacatttaagtattttgtttggtttgaactcaatcagtagcttttccttacatgtttaaaaataattccaatgacagatgagcagctcacttttccaaagtaccccaaaaggccaaattaaaaaagaaaaataatcactctcaagccttgtctaagaaaagaggcaaactctgaaagtcgtaccagtttcttctggaggcaaagcaattttgcacaaaaccagctctctcaagatgagactagaaattcatacctggtcttgtagccacctctctaaacttgaaaataggttcttcttcataagtgagcttacatcattcttcataaagaaaaatcctataacttgttatcatttttgcttcagatactaaaaggcactaagtttccaatttacgctgctcaactttgtttatatgcttaaaaggattctgtttacttaacaattttttcccctaaaatactattttctgaatacttccttccagtaaggaataaaggaaagcccaacttggccataaaattcttgcctacactagaagtttgttgacagccattagctgacttgatcgtcatctcctaagaggaacacatatattttcacaagcaattccacactatcctgatgggtatgcaaagtggtgacagtctaactcagtgtttcttcattttaggtataacattttaaagcaattgataatgcctcttccaattcagaagctagtattgaccaaaatgtgagaagagtgtatagcataggaaaatttggggttaacccaaaagacacaattccagcacacataagaaagctagctgctattttatgctttcttccatggttctcctcttttttcccttttatttttccctgtttttcaatgatgtacagtgttccctacttgcattgaaaaaactcgtatggcattcacactttttttcttaggtgggtttttgtgtccagatgcagtaagaattcattgttcatcctaaaactgttttccagacccttccttccccttaggtaatttgatatacacctcctaaaatgacacagtaacaaatctggtatttagaacatatagaacataaatgccattttttaattcaactttaataagaattacatttgactttggagaatacaggtcttgacccatgtgactgactagctgacccgatcgctgtaatttaacgtcatttataaattctgctgatggacaggaatgtatgaactcaattattgtcagcacaaagccttaaaacctgctgactttaaattaaatggtgcagtcctatgatgccctgcaccatccaggggactaacagggcctcgcagtgtagacagagggtgcagccacacgggcgggggcaccagccacctcactctgcacccgcggcctcacacatctcccagctcacactctactaatgcacagagtcattagatccaatttgttatttttctcacttgctttaaaaaaaagcagtttggataatcatgacattggaataaagtgggaaggaaaaattccatcagcacaaaatagggaagtaatcccaacttgtagtcacagttttctgactggctttgttttaaaagaggatggcagtccttgttcgtgtcagtgtgccactgggtttttgctgttccgtgtaattcatatcaactttgtgttgccatttgcaaggtaaaaggcaaagctgtagtgtattcacctatgtagacagattgctagatatctttttgatctggggcgagttcaatattgattccagacttatttggatttttttagtattattttcccctccctttctaatttaaatagacaaattaagcaaaagtgtgtgttcacaaccaaatgttgatgcccttatctactgataatatcctctcaatgttcactgaggcatagaaattatttcagagtagaaattgcagcatgaggataaactcacctctttgttctgaaaatagaactttatcactatgctttccggtggttttcccttttacaatcgaaatcttgtgcctcccaagtgcattggaaaatgacaaaagcctgtctctccaaattcctatttaacagtttgattttttttttttaatcaccatctttcaaatcttagctcaactctcaccaagtgaaaattggctacttgggagaaagttaactttctatggtgggatggtgaaggatgagggacagtttacataggaaaagaaaaaaaaaagtctaaagtccatgttgaaaaaccacactaccacttattttctgctaaccctaaattatttttgcgtatacgcttgaggttatagtctgtgcctagacctaaaatgcaccagcgggggggattttaaaaaatccttcaaaataccagttttttcccaacaagtacaattgttcttgtgccttctgtggctttcgatttcatctttttgactttatttccaattactacagctgcaataaacactagattttttttctggctgtttgacataacgttgatagctatgcatattttgtgtctttttaaaacaaagcgggagaatacgtttttgaagaagagaatttttagaacagtttgataccgcaaattattttttcctcaattgtttgagcagcattcgagttttgaaaattcttgtagaagccaattttttgtaactgtggtgcaaatcttgtgttttcttagcctaatgaaaagtagtatagaagcaatatttcataccatgtgctatatatgtgtgcgcagatgtgtgaacataaaatcacatacacacatatacacacatgtaaaaatatacatatatatatatgcgtgtgaagtggaaagcttaccttttcctatctagatttaagaacctattttagacatttgttatgttttgtgaaaagaatgttctatttgcaacaaaacatttaattcttactgtatctctggctgtttaatgaggacgtttcacattaaatggtaaaacacatggaagatgttagaatgtagtaattatttaagtaaacgttcacccacatattcctgaagtttgctttgtgcctccgagtattatttaattaaagaagtgttttatgtttgcagaatctttgtcactgtactagggatgtgggtgaatatcatttaaaaaaatttaaaacaacaaaaaaaaagcaaaacagaaacactaaagcaagaggggaacttttataaagcaatgtaaatatttaacctcatggctgtcattatgtaagacatgagattttaataaataactacattctcacgacatctgttgaatttactaggaacactacagtgactgtatagacagttgaaagcattcttgaaaatcctgctctctccttttaaaagttaacaatctcttttatcagatgtcaagggcaagggtaatgcagtttctgtaaatttatgaaatttctttttctatgtacatgaagacatttagtaagtaacacccccccttcccatgcgcacatgtgcgcatacacacacacacacacacacacacacacacaaacacacacactgtcataaagctaatgatttggggactttaaaaaataggatgtcctccaggaacaatcataaatttatgaaagaaagagtagtttacagactcccctgaaagaagcagtgtatatgtgaagacagtgcaaaaatctctttgccatgtatattatagcgtattcattggtgtgaatagtacaaatgtttccttctggtacaaactctgtgtttgcaaatttacaagaagcattgttttcaaaaagctccccttaaaaaatgtaactggtttatatgagtaagcagttaccgtattgcacttaaatgttatgttgaaggaaatgcagttttgttttctgtagatctgttggttgtaaaccatctataaaactaaagctaaaatgctcatattcagagctgggatcaaaactggtatttaacctttgcatcttcttataattatccttctaagaatataacagaatgtggaagtgtctggactttgagtcttttcaactgagccttctctcaaatctgacaccccctcagaatgcacaaacataagcagaaaaggcaaacaagcttaccttcttttgtgaaaacgtattcattctgtatttttttaaatattcaattcccctaaaaatggggagaaaatattttaaaattgtatattacgacttcaaatttagaactaagaaaaaaatgtatttgggattggtctcagcgctacctagaagaatcaaaggtcatggcttccctcaatattgtcccagccatttctcatatgtatatagtataaaccgtgacaaaacactgcctttatattatttagcaatatgttgtaaatagcattattaagctcttttttgtaataaagaccctttgatttgaatatagtacaataactgaactgataaagtcaatttttgatttttgtttgttttttttagctagaggcaatttcaattgtgaatttttgttgttgtctattgttctgaagactttgcataatttattggtttaatttatcctaatttatttgatgaaggtgtacaattttgtattaccaaggatgtactgtaatattaattgatatgataaacacaatgagactccctgtccatattaaaaagaaaataaaaaggtgcagtagacaattgattttaaaggaaaagttaaaaaaattagtttggcagctactaaattttaaaacaggaaaaaaaaaagttgttgtggggagggtgggaaaggggttttactttgtgtgttttaagcttttgtatactctccaaacttttaccttttgctttgtaccacttaaaggatacagtagtccaattgccttgtgtgccttccatctcctcttaaactgaatgtatgtgcagtatatatgcaagcttgtgcaaaataaaatatacattacaagctcagtgccgtttgattttcttaaagaaagagtgacttttaatttttggacctgtatccaattgtaggacagtaggctagttgtgccagtaatgtcaagtatggagattttctttcactacaattcttcattctgttagcctaacgtgcagctcctagaaacaacctcttttactttagatgcttggaataattgcttggatttctctctctgaaacatctttcaggcttaactttatttagccctgaaacttaaaaaaaa

SEQ ID NO: 14 - DNA, 4969 bp, Homo sapiens
gctttttcttaactttcactaagggttactgtagtctgatgtgtccttcccaaggccacgaaatttgacaagctgcacttttcttttgctcaatgatttctgctttaagccaaagaactgcctataatttcactaagaatgtcttctaattcagatactggggatttacaagagtctttaaagcacggacttacacctattggtgctgggcttccggaccgacacggatcccccatccccgcccgcggtcgccttgtcatgctgcccaaagtggagacggaagccctgggactggctcgatcgcatggggaacagggccagatgccggaaaacatgcaagtgtctcaatttaaaatggtgaattactcctatgatgaagatctggaagagctttgtcccgtgtgtggagataaagtgtctgggtaccattatgggctcctcacctgtgaaagctgcaagggattttttaagcgaacagtccaaaataataaaaggtacacatgtatagaaaaccagaactgccaaattgacaaaacacagagaaagcgttgtccttactgtcgttttcaaaaatgtctaagtgttggaatgaagctagaagctgtaagggccgaccgaatgcgtggaggaaggaataagtttgggccaatgtacaagagagacagggccctgaagcaacagaaaaaagccctcatccgagccaatggacttaagctagaagccatgtctcaggtgatccaagctatgccctctgacctgaccatttcctctgcaattcaaaacatccactctgcctccaaaggcctacctctgaaccatgctgccttgcctcctacagactatgacagaagtccctttgtaacatcccccattagcatgacaatgccccctcacggcagcctgcaaggttaccaaacatatggccactttcctagccgggccatcaagtctgagtacccagacccctataccagctcacccgagtccataatgggctattcatatatggatagttaccagacgagctctccagcaagcatcccacatctgatactggaacttttgaagtgtgagccagatgagcctcaagtccaggctaaaatcatggcctatttgcagcaagagcaggctaaccgaagcaagcacgaaaagctgagcacctttgggcttatgtgcaaaatggcagatcaaactctcttctccattgtcgagtgggccaggagtagtatcttcttcagagaacttaaggttgatgaccaaatgaagctgcttcagaactgctggagtgagctcttaatcctcgaccacatttaccgacaagtggtacatggaaaggaaggatccatcttcctggttactgggcaacaagtggactattccataatagcatcacaagccggagccaccctcaacaacctcatgagtcatgcacaggagttagtggcaaaacttcgttctctccagtttgatcaacgagagttcgtatgtctgaaattcttggtgctctttagtttagatgtcaaaaaccttgaaaacttccagctggtagaaggtgtccaggaacaagtcaatgccgccctgctggactacacaatgtgtaactacccgcagcagacagagaaatttggacagctacttcttcgactacccgaaatccgggccatcagtatgcaggctgaagaatacctctactacaagcacctgaacggggatgtgccctataataaccttctcattgaaatgttgcatgccaaaagagcataagttacaacccctaggagctctgctttcaaaacaaaaagagattgggggagtggggagggggaagaagaacaggaagaaaaaaagtactctgaactgctccaagtaacgctaattaaaaacttgctttaaagatattgaatttaaaaaggcataataatcaaatacttaatagcaaataaatgatgtatcagggtatttgtattgcaaactgtgaatcaaaggcttcacagccccagaggattccatataaaagacattgtaatggagtggattgaactcacagatggataccaacacggtcagaagaaaaacggacagaacggttcttgtatatttaaactgatctccactatgaagaaatttaggaactaatcttattaattaggcttatacagcgggggatttgagcttacaggattcctccatggtaaagctgaactgaaacaattctcaagaatgcatcagctgtacctacaatagcccctccctcttcctttgaaggccccagcacctctgccctgtggtcaccgaatctgtactaaggacctgtgttcagccacacccagtggtagctccaccaaatcatgaacagcctaattttgagtgtctgtgtcttagacctgcaaacagctaataggaaattctattaatatgttagcttgccattttaaatatgttctgagggttgttttgtctcgtgttcatgatgttaagaaaatgcaggcagtatccctcatcttatgtaagtgttaattaatattaagggaaatgactacaaactttcaaagcaaatgctccatagctaaagcaacttagaccttatttctgctactgttgctgaaatgtggctttggcattgttggatttcataaaaaatttctggcaggaagtcttgttagtatacatcagtctttttcatcatccaagtttgtagttcatttaaaaatacaacattaaacacattttgctaggatgtcaaatagtcacagttctaagtagttggaaacaaaattgacgcatgttaatctatgcaaagagaaaggaaaggatgaggtgatgtattgactcaaggttcattcttgctgcaattgaacatcctcaagagttgggatggaaatggtgatttttacatgtgtcctggaaagatattaaagtaattcaaatcttccccaaaggggaaaggaagagagtgatactgacctttttaagtcatagaccaaagtctgctgtagaacaaatatgggaggacaaagaatcgcaaattcttcaaatgactattatcagtattattaacatgcgatgccacaggtatgaaagtcttgccttatttcacaattttaaaaggtagctgtgcagatgtggatcaacatttgtttaaaataaagtattaatactttaaagtcaaataagatatagtgtttacattctttaggtcctgaggggcagggggatctgtgatataacaaaatagcaaaagcggtaatttccttaatgttatttttctgattggtaattatttttaacagtacttaattattctatgtcgtgagacactaaaatcaaaaacgggaatctcatttagactttaatttttttgagattatcggcggcacaatcactttgtagaaactgtaaaaaataaaagtatctcctagtcccttaattttttcataaatatttctggcttttgagtagtgtatttatattgtatatcatactttcaactgtagacaattatgatgctaatttattgtttcttggtttcacctttgtataagatatagccaagactgaagaaaccaaatatatgtgtttactgtagcatgtcttcaaattagtggaacttagttcagggacatagaagagtcttaatgaattaaaatcattcacttgattaaatgtctgtaaatcttcatcattcctactgtagtttatttaatatctattgtaaattatgtgacttgtagcttcctctggttttcaagtaaactcaacaaggtggagtcttacctggttttcctttccaagcattgtaaattgtataccaaagatattagttattacttctgtgtgtacaaagaggattattttattatgtttattaatcacctctaatactcatccacatgaagggtacacattaggtaagctgggcgttgactcatgcgcagtctcagtcacccgtgttatcttcgtggctcaaaggacaatgcaaaatcgccgatcagagctcatacccaaagcattacagagaacagcagcatcattgccctccccagctgaaaaacaagttggctagaagatacatggagaggaatggtgtggtcaacagttaatgaaacggttctatcatgcatgtgtaatgtggatggagacaattataagatttgactataactatttggagggtctttaacattgccaaaaaaacaaatatgttgatttttattttattttattttttattttaagaggcgggatcttgatctcacatgttgcccaggctggccttgaactcctgggctcaagcattcctcctgcctcagcctcccccatagctgggactaggggtgcatgccagcatacctggctacgttgactcttaaaatctatgttctcttattttaaagatacagtgctccccactgaaaattaaacctaaaaaatgtcacatattggtatgttgttaacctggtagattaaatcatgagaatgattagaaagacgggcaacacagcgggttacatccacactgctgatcacaccaacgacaggagctgataagcaagaaagcgtcacagccagcgtctgttcacccaaggttgacaagtgaagtttctctaatgttgattgttagccgatttgtaacctggcatttacttagcaactgccttatcaattacaggatttgccggtaaaagcagactcaaatataaaggtttttggcttaacttggtttattatagttgctctatgtttgtaaacagacaatctctaatgtctgattatttgtatcacagatctgcagctgccttggacttgaatccatgcaatgtttagagtgtgaagtcagttacttgttgatgttttcttactgtatcaatgaaatacatattgtcatgtcagttcttgccaggaacttctcaacaaaatggaattttttttttcagtatttcaataaatattgatatgcccagcctgataa

SEQ ID NO: 15 - DNA, 4831 bp, Homo sapiens
gctttttcttaactttcactaagggttactgtagtctgatgtgtccttcccaaggccacgaaatttgacaagctgcacttttcttttgctcaatgatttctgctttaagccaaagaactgcctataatttcactaagaatgtcttctaattcagatactggggatttacaagagtctttaaagcacggacttacacctattgtgtctcaatttaaaatggtgaattactcctatgatgaagatctggaagagctttgtcccgtgtgtggagataaagtgtctgggtaccattatgggctcctcacctgtgaaagctgcaagggattttttaagcgaacagtccaaaataataaaaggtacacatgtatagaaaaccagaactgccaaattgacaaaacacagagaaagcgttgtccttactgtcgttttcaaaaatgtctaagtgttggaatgaagctagaagctgtaagggccgaccgaatgcgtggaggaaggaataagtttgggccaatgtacaagagagacagggccctgaagcaacagaaaaaagccctcatccgagccaatggacttaagctagaagccatgtctcaggtgatccaagctatgccctctgacctgaccatttcctctgcaattcaaaacatccactctgcctccaaaggcctacctctgaaccatgctgccttgcctcctacagactatgacagaagtccctttgtaacatcccccattagcatgacaatgccccctcacggcagcctgcaaggttaccaaacatatggccactttcctagccgggccatcaagtctgagtacccagacccctataccagctcacccgagtccataatgggctattcatatatggatagttaccagacgagctctccagcaagcatcccacatctgatactggaacttttgaagtgtgagccagatgagcctcaagtccaggctaaaatcatggcctatttgcagcaagagcaggctaaccgaagcaagcacgaaaagctgagcacctttgggcttatgtgcaaaatggcagatcaaactctcttctccattgtcgagtgggccaggagtagtatcttcttcagagaacttaaggttgatgaccaaatgaagctgcttcagaactgctggagtgagctcttaatcctcgaccacatttaccgacaagtggtacatggaaaggaaggatccatcttcctggttactgggcaacaagtggactattccataatagcatcacaagccggagccaccctcaacaacctcatgagtcatgcacaggagttagtggcaaaacttcgttctctccagtttgatcaacgagagttcgtatgtctgaaattcttggtgctctttagtttagatgtcaaaaaccttgaaaacttccagctggtagaaggtgtccaggaacaagtcaatgccgccctgctggactacacaatgtgtaactacccgcagcagacagagaaatttggacagctacttcttcgactacccgaaatccgggccatcagtatgcaggctgaagaatacctctactacaagcacctgaacggggatgtgccctataataaccttctcattgaaatgttgcatgccaaaagagcataagttacaacccctaggagctctgctttcaaaacaaaaagagattgggggagtggggagggggaagaagaacaggaagaaaaaaagtactctgaactgctccaagtaacgctaattaaaaacttgctttaaagatattgaatttaaaaaggcataataatcaaatacttaatagcaaataaatgatgtatcagggtatttgtattgcaaactgtgaatcaaaggcttcacagccccagaggattccatataaaagacattgtaatggagtggattgaactcacagatggataccaacacggtcagaagaaaaacggacagaacggttcttgtatatttaaactgatctccactatgaagaaatttaggaactaatcttattaattaggcttatacagcgggggatttgagcttacaggattcctccatggtaaagctgaactgaaacaattctcaagaatgcatcagctgtacctacaatagcccctccctcttcctttgaaggccccagcacctctgccctgtggtcaccgaatctgtactaaggacctgtgttcagccacacccagtggtagctccaccaaatcatgaacagcctaattttgagtgtctgtgtcttagacctgcaaacagctaataggaaattctattaatatgttagcttgccattttaaatatgttctgagggttgttttgtctcgtgttcatgatgttaagaaaatgcaggcagtatccctcatcttatgtaagtgttaattaatattaagggaaatgactacaaactttcaaagcaaatgctccatagctaaagcaacttagaccttatttctgctactgttgctgaaatgtggctttggcattgttggatttcataaaaaatttctggcaggaagtcttgttagtatacatcagtctttttcatcatccaagtttgtagttcatttaaaaatacaacattaaacacattttgctaggatgtcaaatagtcacagttctaagtagttggaaacaaaattgacgcatgttaatctatgcaaagagaaaggaaaggatgaggtgatgtattgactcaaggttcattcttgctgcaattgaacatcctcaagagttgggatggaaatggtgatttttacatgtgtcctggaaagatattaaagtaattcaaatcttccccaaaggggaaaggaagagagtgatactgacctttttaagtcatagaccaaagtctgctgtagaacaaatatgggaggacaaagaatcgcaaattcttcaaatgactattatcagtattattaacatgcgatgccacaggtatgaaagtcttgccttatttcacaattttaaaaggtagctgtgcagatgtggatcaacatttgtttaaaataaagtattaatactttaaagtcaaataagatatagtgtttacattctttaggtcctgaggggcagggggatctgtgatataacaaaatagcaaaagcggtaatttccttaatgttatttttctgattggtaattatttttaacagtacttaattattctatgtcgtgagacactaaaatcaaaaacgggaatctcatttagactttaatttttttgagattatcggcggcacaatcactttgtagaaactgtaaaaaataaaagtatctcctagtcccttaattttttcataaatatttctggcttttgagtagtgtatttatattgtatatcatactttcaactgtagacaattatgatgctaatttattgtttcttggtttcacctttgtataagatatagccaagactgaagaaaccaaatatatgtgtttactgtagcatgtcttcaaattagtggaacttagttcagggacatagaagagtcttaatgaattaaaatcattcacttgattaaatgtctgtaaatcttcatcattcctactgtagtttatttaatatctattgtaaattatgtgacttgtagcttcctctggttttcaagtaaactcaacaaggtggagtcttacctggttttcctttccaagcattgtaaattgtataccaaagatattagttattacttctgtgtgtacaaagaggattattttattatgtttattaatcacctctaatactcatccacatgaagggtacacattaggtaagctgggcgttgactcatgcgcagtctcagtcacccgtgttatcttcgtggctcaaaggacaatgcaaaatcgccgatcagagctcatacccaaagcattacagagaacagcagcatcattgccctccccagctgaaaaacaagttggctagaagatacatggagaggaatggtgtggtcaacagttaatgaaacggttctatcatgcatgtgtaatgtggatggagacaattataagatttgactataactatttggagggtctttaacattgccaaaaaaacaaatatgttgatttttattttattttattttttattttaagaggcgggatcttgatctcacatgttgcccaggctggccttgaactcctgggctcaagcattcctcctgcctcagcctcccccatagctgggactaggggtgcatgccagcatacctggctacgttgactcttaaaatctatgttctcttattttaaagatacagtgctccccactgaaaattaaacctaaaaaatgtcacatattggtatgttgttaacctggtagattaaatcatgagaatgattagaaagacgggcaacacagcgggttacatccacactgctgatcacaccaacgacaggagctgataagcaagaaagcgtcacagccagcgtctgttcacccaaggttgacaagtgaagtttctctaatgttgattgttagccgatttgtaacctggcatttacttagcaactgccttatcaattacaggatttgccggtaaaagcagactcaaatataaaggtttttggcttaacttggtttattatagttgctctatgtttgtaaacagacaatctctaatgtctgattatttgtatcacagatctgcagctgccttggacttgaatccatgcaatgtttagagtgtgaagtcagttacttgttgatgttttcttactgtatcaatgaaatacatattgtcatgtcagttcttgccaggaacttctcaacaaaatggaattttttttttcagtatttcaataaatattgatatgcccagcctgataa

SEQ ID NO: 16 - DNA, 4787 bp, Homo sapiens
atagtttgtcatctttttagctgcgaagacgccttcgtgggtctgcgtccggagcaaggcggttacccgatcccggcagtgagccgcgccgcgcgtcgtggcggcctgatttctgtttaactttaatagggcgatctcgagtgtctcaatttaaaatggtgaattactcctatgatgaagatctggaagagctttgtcccgtgtgtggagataaagtgtctgggtaccattatgggctcctcacctgtgaaagctgcaagggattttttaagcgaacagtccaaaataataaaaggtacacatgtatagaaaaccagaactgccaaattgacaaaacacagagaaagcgttgtccttactgtcgttttcaaaaatgtctaagtgttggaatgaagctagaagctgtaagggccgaccgaatgcgtggaggaaggaataagtttgggccaatgtacaagagagacagggccctgaagcaacagaaaaaagccctcatccgagccaatggacttaagctagaagccatgtctcaggtgatccaagctatgccctctgacctgaccatttcctctgcaattcaaaacatccactctgcctccaaaggcctacctctgaaccatgctgccttgcctcctacagactatgacagaagtccctttgtaacatcccccattagcatgacaatgccccctcacggcagcctgcaaggttaccaaacatatggccactttcctagccgggccatcaagtctgagtacccagacccctataccagctcacccgagtccataatgggctattcatatatggatagttaccagacgagctctccagcaagcatcccacatctgatactggaacttttgaagtgtgagccagatgagcctcaagtccaggctaaaatcatggcctatttgcagcaagagcaggctaaccgaagcaagcacgaaaagctgagcacctttgggcttatgtgcaaaatggcagatcaaactctcttctccattgtcgagtgggccaggagtagtatcttcttcagagaacttaaggttgatgaccaaatgaagctgcttcagaactgctggagtgagctcttaatcctcgaccacatttaccgacaagtggtacatggaaaggaaggatccatcttcctggttactgggcaacaagtggactattccataatagcatcacaagccggagccaccctcaacaacctcatgagtcatgcacaggagttagtggcaaaacttcgttctctccagtttgatcaacgagagttcgtatgtctgaaattcttggtgctctttagtttagatgtcaaaaaccttgaaaacttccagctggtagaaggtgtccaggaacaagtcaatgccgccctgctggactacacaatgtgtaactacccgcagcagacagagaaatttggacagctacttcttcgactacccgaaatccgggccatcagtatgcaggctgaagaatacctctactacaagcacctgaacggggatgtgccctataataaccttctcattgaaatgttgcatgccaaaagagcataagttacaacccctaggagctctgctttcaaaacaaaaagagattgggggagtggggagggggaagaagaacaggaagaaaaaaagtactctgaactgctccaagtaacgctaattaaaaacttgctttaaagatattgaatttaaaaaggcataataatcaaatacttaatagcaaataaatgatgtatcagggtatttgtattgcaaactgtgaatcaaaggcttcacagccccagaggattccatataaaagacattgtaatggagtggattgaactcacagatggataccaacacggtcagaagaaaaacggacagaacggttcttgtatatttaaactgatctccactatgaagaaatttaggaactaatcttattaattaggcttatacagcgggggatttgagcttacaggattcctccatggtaaagctgaactgaaacaattctcaagaatgcatcagctgtacctacaatagcccctccctcttcctttgaaggccccagcacctctgccctgtggtcaccgaatctgtactaaggacctgtgttcagccacacccagtggtagctccaccaaatcatgaacagcctaattttgagtgtctgtgtcttagacctgcaaacagctaataggaaattctattaatatgttagcttgccattttaaatatgttctgagggttgttttgtctcgtgttcatgatgttaagaaaatgcaggcagtatccctcatcttatgtaagtgttaattaatattaagggaaatgactacaaactttcaaagcaaatgctccatagctaaagcaacttagaccttatttctgctactgttgctgaaatgtggctttggcattgttggatttcataaaaaatttctggcaggaagtcttgttagtatacatcagtctttttcatcatccaagtttgtagttcatttaaaaatacaacattaaacacattttgctaggatgtcaaatagtcacagttctaagtagttggaaacaaaattgacgcatgttaatctatgcaaagagaaaggaaaggatgaggtgatgtattgactcaaggttcattcttgctgcaattgaacatcctcaagagttgggatggaaatggtgatttttacatgtgtcctggaaagatattaaagtaattcaaatcttccccaaaggggaaaggaagagagtgatactgacctttttaagtcatagaccaaagtctgctgtagaacaaatatgggaggacaaagaatcgcaaattcttcaaatgactattatcagtattattaacatgcgatgccacaggtatgaaagtcttgccttatttcacaattttaaaaggtagctgtgcagatgtggatcaacatttgtttaaaataaagtattaatactttaaagtcaaataagatatagtgtttacattctttaggtcctgaggggcagggggatctgtgatataacaaaatagcaaaagcggtaatttccttaatgttatttttctgattggtaattatttttaacagtacttaattattctatgtcgtgagacactaaaatcaaaaacgggaatctcatttagactttaatttttttgagattatcggcggcacaatcactttgtagaaactgtaaaaaataaaagtatctcctagtcccttaattttttcataaatatttctggcttttgagtagtgtatttatattgtatatcatactttcaactgtagacaattatgatgctaatttattgtttcttggtttcacctttgtataagatatagccaagactgaagaaaccaaatatatgtgtttactgtagcatgtcttcaaattagtggaacttagttcagggacatagaagagtcttaatgaattaaaatcattcacttgattaaatgtctgtaaatcttcatcattcctactgtagtttatttaatatctattgtaaattatgtgacttgtagcttcctctggttttcaagtaaactcaacaaggtggagtcttacctggttttcctttccaagcattgtaaattgtataccaaagatattagttattacttctgtgtgtacaaagaggattattttattatgtttattaatcacctctaatactcatccacatgaagggtacacattaggtaagctgggcgttgactcatgcgcagtctcagtcacccgtgttatcttcgtggctcaaaggacaatgcaaaatcgccgatcagagctcatacccaaagcattacagagaacagcagcatcattgccctccccagctgaaaaacaagttggctagaagatacatggagaggaatggtgtggtcaacagttaatgaaacggttctatcatgcatgtgtaatgtggatggagacaattataagatttgactataactatttggagggtctttaacattgccaaaaaaacaaatatgttgatttttattttattttattttttattttaagaggcgggatcttgatctcacatgttgcccaggctggccttgaactcctgggctcaagcattcctcctgcctcagcctcccccatagctgggactaggggtgcatgccagcatacctggctacgttgactcttaaaatctatgttctcttattttaaagatacagtgctccccactgaaaattaaacctaaaaaatgtcacatattggtatgttgttaacctggtagattaaatcatgagaatgattagaaagacgggcaacacagcgggttacatccacactgctgatcacaccaacgacaggagctgataagcaagaaagcgtcacagccagcgtctgttcacccaaggttgacaagtgaagtttctctaatgttgattgttagccgatttgtaacctggcatttacttagcaactgccttatcaattacaggatttgccggtaaaagcagactcaaatataaaggtttttggcttaacttggtttattatagttgctctatgtttgtaaacagacaatctctaatgtctgattatttgtatcacagatctgcagctgccttggacttgaatccatgcaatgtttagagtgtgaagtcagttacttgttgatgttttcttactgtatcaatgaaatacatattgtcatgtcagttcttgccaggaacttctcaacaaaatggaattttttttttcagtatttcaataaatattgatatgcccagcctgataatttttaaaaaaaaaaaa

SEQ ID NO: 17 - DNA, 1165 bp, Homo sapiens
gccagagagctggaagtgagagcagatccctaaccatgagcaccagccaaccaggggcctgcccatgccagggagctgcaagccgccccgccattctctacgcacttctgagctccagcctcaaggctgtcccccgaccccgtagccgctgcctatgtaggcagcaccggcccgtccagctatgtgcacctcatcgcacctgccgggaggccttggatgttctggccaagacagtggccttcctcaggaacctgccatccttctggcagctgcctccccaggaccagcggcggctgctgcagggttgctggggccccctcttcctgcttgggttggcccaagatgctgtgacctttgaggtggctgaggccccggtgcccagcatactcaagaagattctgctggaggagcccagcagcagtggaggcagtggccaactgccagacagaccccagccctccctggctgcggtgcagtggcttcaatgctgtctggagtccttctggagcctggagcttagccccaaggaatatgcctgcctgaaagggaccatcctcttcaaccccgatgtgccaggcctccaagccgcctcccacattgggcacctgcagcaggaggctcactgggtgctgtgtgaagtcctggaaccctggtgcccagcagcccaaggccgcctgacccgtgtcctcctcacggcctccaccctcaagtccattccgaccagcctgcttggggacctcttctttcgccctatcattggagatgttgacatcgctggccttcttggggacatgcttttgctcaggtgacctgttccagcccaggcagagatcaggtgggcagaggctggcagtgctgattcagcctggccatccccagaggtgacccaatgctcctggagggggcaagcctgtatagacagcacttggctccttaggaacagctcttcactcagccacaccccacattggacttccttggtttggacacagtgttccagctgcctgggaggcttttggtggtccccacagcctctgggccaagactcctgtcccttcttgggatgagaatgaaagcttaggctgcttattggaccagaagtcctatcgactttatacagaactgaattaagttattgatttttgtaataaaaggtatgaaacac

SEQ ID NO: 18 - DNA, 7937 bp, Homo sapiens
aatcatggtgccgctggggaggggagaagctgctgctgccgccgttgccgggagccgcggagacaagtcattacgttttcatttctcacaactgggctgagcacaactgaaccatgggggaacacagtccagacaacaacatcatctactttgaggcagaggaagatgagctgacccccgatgataaaatgctcaggtttgtggataaaaacggactggtgccttcctcatctggaactgtttatgataggaccactgttcttattgagcaggaccctggcactttggaggatgaagatgacgacggacagtgcggagaacacttgccttttctagtagggggtgaagagggctttcacctgatagatcatgaagcaatgtcccagggttatgtgcagcacattatctcaccagatcagattcatttgacaataaaccctggttccacacccatgccaagaaatattgaaggtgcaaccctcactctgcagtcggaatgtccggaaacaaaacgtaaagaagtaaagcggtaccaatgtacctttgagggctgtccccgcacctacagcacagcaggcaacctgcgaacccaccagaagactcaccgaggagagtacacctttgtctgtaatcaggagggctgtggcaaagccttccttacctcttacagcctcaggatccacgtgcgagtgcacacgaaggagaagccatttgagtgtgacgtgcagggctgtgagaaggcattcaacacactgtacaggctgaaagcacatcagaggcttcacacagggaaaacgtttaactgtgaatctgaaggctgcagcaaatacttcaccacactcagtgatctgaggaagcacattcgaactcatacaggggaaaagccatttcggtgcgatcacgatggctgtggaaaagcatttgcagcaagccaccaccttaaaactcacgttcgtacacatactggtgaaagacccttcttctgccccagtaatggctgtgagaaaacattcagcactcaatacagtctcaaaagtcacatgaaaggtcatgataacaaaggacactcatacaatgcacttccacaacacaatggatcagaggatacaaatcactcactttgtctaagtgacttgagccttctgtccacagattctgaattgcgagaaaattccagtacgacccagggccaggacctcagcacaatttcaccagcaatcatctttgaatcaatgttccagaattcagatgatacggcaattcaggaagatcctcaacagacagcttccttgactgaaagttttaatggtgatgcagagtcagtcagtgatgttccgccatccacaggaaattcagcatctttatctcttccacttgtactgcaacctggcctctccgagccaccccagcctctactacctgcctcagctccgtctgctcctccgcctgctccctccctaggacctggctcccagcaagctgcatttggcaacccccctgctctcttacaacctccagaagtgcctgttccccacagcacacagtttgctgctaatcatcaagagtttcttccgcacccccaggcaccgcagcccattgtaccaggactttctgttgttgctggggcttctgcatcagcagcggcagtggcatcagctgtggcagcaccagccccaccacaaagtactactgagcccctgccagccatggtccagactctgcccctgggtgccaactctgtcctaactaataatcccacaataaccatcaccccaactcccaacacagctatcctgcagtccagcctagtcatgggagaacagaacttacaatggatattaaatggtgccaccagttctccacaaaaccaagaacaaattcagcaagcatctaaagttgagaaggtgttttttaccactgcagtaccagtagccagtagcccagggagctctgtccagcagattggcctcagtgttcctgtgatcatcatcaaacaagaagaggcatgtcagtgtcagtgtgcatgccgggactctgcaaaggagcgggcatccagcaggagaaagggctgctcctccccaccccctccagagccgagcccccaggctcctgatgggcccagcctgcagctcccagcgcagactttctcttcagcccctgttcccgggtcatcatcctctaccttgccctcctcctgtgagcaaagccgacaagcagagactccttcagaccctcagacagaaacattaagtgccatggatgtgtcagagtttctatccctccagagcctggacaccccgtccaatctgattcccattgaagcactactgcagggggaggaggagatgggcctcaccagcagcttctccaagtgaagggcccatgtgtgctcacctctgggaaaagcgggtgagcaggaggcatgaggtacaatgcctgccatcatgggtcagaaatttgaaggatgaagaaatctactgtttgaaatcctcacctttcagacgtattttctttattcacatcccaggagcatccattttaaggaactattctttggaaaaaaacaaaaaacaaaaaaaacaacaaaaaaagctaagttataagtgaactgtttggctgcactgtatgtcacttttgcttgttgtcatgtgaacttggaaactaaggttactcgtgtgcataaaaattctaaatgaaagggtgtggtttccatcaatctgatgctgcccatcgcttgcactggggtctttgtggatcgggcaggagttttcagtgtgttgggtgttgctccttcctatgtgtcttttgaatctgaggctgacatttgcttggaaggccagacccttgctccatcagagagggcagtggcaaaggccagtgaggcagctgtgagttggacagggttcaggtgagatggtgttgtcatttgtgcttagtgttggtggtgctcagggtggataacacgggtcgttctgcagcccgcttcagcacaaataggcagcttaaggcctggctcacaggctgtggggttgatctggctctgcagaggccctaggcagcttgttgactgctgtctgttgatgacgtgtgtgcaaagcaggctctagcaacatgatcactgtccttgccttcctggttctttctctcggttggttgccagggcttgcagatcgcagtgaattttccttggggaacatcgctgttttgtcctagagtgaacttgtggcttatggccagtgctgtttggtggtctgccttctttttaatggtattttcttcctcagagcagaagggctgcattttgcttatcagaagaaggtgcagatttaagggaattcatatgaggtggcatgtaattggcaggccaggtgtcctggttccaggttccagccaggctttgggttgccccctccatctctgcccccctctggattttgcatacagcctcatacagtgcaaacaaggatgtgacttgctcagcttagtcatgtgatttatttaaaaaaaaaaaaaaaagaaacacaaaacgatgatcttctactcagggtatagcaaaacaaaaaaattccctttccaccaaaaagcctgaaatgttgcaataagttatctcatttggaatgtttcattaagttgtgttataggaaaaaattgtgtgtgtgtgttatagaattatatccatatgtctgcctttggctccaagtcattgcctcttaaaataaaagatacaatccatactagcatgaaaagtttccctcaacaggctatattaacatagtcatgagtgctgaccaaactcaccgagctcagaggccaggcatggcctgaggtgcagaataggcctctgcctcccaagagccctttccttgccctgagcaaggagtggtgttccacaaacaaggctgctcttctaagccaacagtgtcaggcaggaagcagccataattttgccttgcattttcattccctaatgtaaagggatctgcattggtcactctcctgttctctgagccattgctcagggccagccaagatattattgagaacagataatttaccttggagccagaggccctccctgcctttagcaaggatgttcagggacagacaaagagggcagtggtggtgaatgttgttactgccatgaggagaaatggcagtaagaaatcttaactacaagcagccaatttctcattccaggaccctagccagaataattgacttctttttttttttgagacagagttttgcttttgttgccctggctggagtgcagtggcgcaatcttggctcaccgcaacctccacttcccaggttcaagcaattctgcctcagcctcccgagtagctgggattacaggcatgcgccaccacgcctggctaattttgtatttttagtagagacggggtttctccatgttggtcaggctggtctcgaactcccaacgtcaggtgatcctcccgcctcagtctcccaaagtgctgggattacaggtgtgagccaccatgcctggtctagggaattgacttctttttgaccttctgcactcccttccccaaaaggattgtggcttctgttgaccattgacctcagcagaagttgataaggcaggaggtttccagtcctcctggaaaaccaactgctgagcatgagttgtccttggctgtctctgggcctcgcacccatgggaagttttgaggtagggcccttgctgtttacaacttctgagagagtagtgatgggaccccaaagtaagcttgtatcagagggcagtaatgaccttcccatatccccatcctgtggtcacctgggatttgggttccctggggcggaagtgggaaataggagcccagggaaggacatctgaagcacccacagtttaaatagcgactcttcttggttagccagggctgtgctcatgttgggccctacccaggtcagtggccttttcactctcaaagatcgggggtgacgaagcagcatcttaaacagtgtttaggctgacagatttttccagttgaagaagctgaagtatctgccttttgagggtgactctagattacaagagagactatcagatccaatcagcttttgaaaaatcaggaatgtgcttaaaatgccagtgagtggttgtgaagatcaggtttatttcccaggataggcagtctttctttccctccttttccagttctcttttcctacctgtccttccataagcctggtctctagtgccgaggaccttggagaagagagggcccctagctaagctggagccagaaagaaccttcatggtgagtgtggtttcccaaacttgggaaatgaacctagggtgaggttagggaggatttgtagaattctagcaggtaaaaatcaagcctttttcccctcccatagtaaacttaaccattttcaccctgtacgttatcttctagctcctactaacatctccaattagacaaccgcttcaaagggtgcttagcaccagggattggggttcatggacttaggaggtggtaaggaaactggctggcaccaccaaactgccttagtgaacttggcccttcccaggcaggtaggttgggcattgagggaaggtggcccagtctttgctggcagggtctggccagtctcatgggggcaccaccagtttggtgacagaagtggtgtcatttattgaattccacctccgtttagaaggagatcatggtacaaagcccaggaggggccttaagatgtgaagatctctctgagtaaaaggcaaggggcttctcttcttcacctctgggatagttggtagatcggagagttttattttcagggtcaaggctgtggactgatggggatattggagggtgggtgggttttcctgagagactttgtataatgctgaatgtgtccagagggacaagtttgcagaacctcatattggtatattaaagaaataataaaataaaaaagcactttaggttattttatctttaacccgattgctgcaatttcttttgtgtgtatatatacatatatatactttccacaaagttttattttttgctcagaataaaaagttaaattgaggtgtgaaaagaaaagcacttaccttggtgcaatatgtgtagcttgatggtcgttgtcccatgtggccctggcctggcagcgtttttccgctcaatcagccctgtgctgtgagactgtccatagggaaacactattatgcattctcagcaaccgctcaatctatgcaagccttccctgtgtgccccagggcgccccctcaggctctctgaagaactgctgtgggtcctgttttctgctgactgttgaggccctttttcatcacttcttggtctctcgccatcttttccctcttcaccattacaaaatgatgcctgaaaggaaggaacagattgttcctaggtagaaacctggcaccttctagacttttatatttgtaatcacatccattgtccttaaagacttttccagagtgattgaaaccattgatttgtggaactgcaacaatatttctcaagagtttacaattgtcttacaccaccaatcagaaatatgtttggggaggcatgggtggcagggggcaacatgagccatttccatactgctccccatactacctgtgttggttccttaagcaaaaaggcctccagccttcattaaatcctataaacaaacattacatcccatgaatcattatcaactttcttcctttactccacctcccccattttatcttcaattctcaggctatagaatagacaagttttagaatatacttcagccaaagcagaaggattttcatagatccaatatgcaaatagttctgctgtgacatagatcagaaattgtctgttccttaatatttgggggtggaggcaacaaaactgaagcatatttctgattggttagccattttggcctgtttcctcttgcatgttttttagggagagagggaaacagtcctgtatttcttctgatgccctttggggaagccgacgaccactgggcatttctcactgttactcctgttcaagagagggcttctcagtctgcactgaaaaatgcaaattaaactggatctttatgtcaatgtgtacatagtacaagcttttttactggaattgaggtttaaaaccacacactgcccttttggtggtgtgcctgttgggccaaaaattgggtgataatgtagtgtcactttctcagctcaatgcagtttctactttttcttatgggaaaatttttcataaaacctttttgcaccaaaacccaggggtgttttttgcaatatccttgttatcctcgtagtgtgccaagtcagaggctttctcttgcccttttcctgctgtgttctcaggcctcccaagggctatttgactcaacagtctacatccttcgttgtgttttggagaatgtgggggtgggggtcagagttcaaggtgtctgttcccttttcctgtgaactctttctagtccctatttggggagggtggctggaaacagatttttgctgaatttctggctcagatcttctagccaggaaaggcaagagcccccaagagcccttttttttgacatacactaatcattggccggggtcttggtgacaacttttaaaatcccaaatagttttatttggattatgtaaaagtaagtgtgaaacatgggaacaacggacttccactgagcgatgtgaaaacgttacaggttcagtacttccaaaggaagaaacctccaaacccaaaaaagaataaatatgaatttgtatttttgaagaatgtgaaataatggtgtttgcttaattgctcattttgtataaacttaatattgtactttaaaatatctgctaaaaagtgaaaatttaactttttggaattgaaaaagcaatattaaatactaatgaaatcctaattaaatgcttatttaaa

SEQ ID NO: 19 - DNA, 4991 bp, Homo sapiens
agtttccgaggaacttttcgccggcgccgggccgcctctgaggccagggcaggacacgaacgcgcggagcggcggcggcgactgagagccggggccgcggcggcgctccctaggaagggccgtacgaggcggcgggcccggcgggcctcccggaggaggcggctgcgccatggacgagccacccttcagcgaggcggctttggagcaggcgctgggcgagccgtgcgatctggacgcggcgctgctgaccgacatcgaaggtgaagtcggcgcggggaggggtagggccaacggcctggacgccccaagggcgggcgcagatcgcggagccatggattgcactttcgaagacatgcttcagcttatcaacaaccaagacagtgacttccctggcctatttgacccaccctatgctgggagtggggcagggggcacagaccctgccagccccgataccagctccccaggcagcttgtctccacctcctgccacattgagctcctctcttgaagccttcctgagcgggccgcaggcagcgccctcacccctgtcccctccccagcctgcacccactccattgaagatgtacccgtccatgcccgctttctcccctgggcctggtatcaaggaagagtcagtgccactgagcatcctgcagacccccaccccacagcccctgccaggggccctcctgccacagagcttcccagccccagccccaccgcagttcagctccacccctgtgttaggctaccccagccctccgggaggcttctctacaggaagccctcccgggaacacccagcagccgctgcctggcctgccactggcttccccgccaggggtcccgcccgtctccttgcacacccaggtccagagtgtggtcccccagcagctactgacagtcacagctgcccccacggcagcccctgtaacgaccactgtgacctcgcagatccagcaggtcccggtcctgctgcagccccacttcatcaaggcagactcgctgcttctgacagccatgaagacagacggagccactgtgaaggcggcaggtctcagtcccctggtctctggcaccactgtgcagacagggcctttgccgaccctggtgagtggcggaaccatcttggcaacagtcccactggtcgtagatgcggagaagctgcctatcaaccggctcgcagctggcagcaaggccccggcctctgcccagagccgtggagagaagcgcacagcccacaacgccattgagaagcgctaccgctcctccatcaatgacaaaatcattgagctcaaggatctggtggtgggcactgaggcaaagctgaataaatctgctgtcttgcgcaaggccatcgactacattcgctttctgcaacacagcaaccagaaactcaagcaggagaacctaagtctgcgcactgctgtccacaaaagcaaatctctgaaggatctggtgtcggcctgtggcagtggagggaacacagacgtgctcatggagggcgtgaagactgaggtggaggacacactgaccccacccccctcggatgctggctcacctttccagagcagccccttgtcccttggcagcaggggcagtggcagcggtggcagtggcagtgactcggagcctgacagcccagtctttgaggacagcaaggcaaagccagagcagcggccgtctctgcacagccggggcatgctggaccgctcccgcctggccctgtgcacgctcgtcttcctctgcctgtcctgcaaccccttggcctccttgctgggggcccgggggcttcccagcccctcagataccaccagcgtctaccatagccctgggcgcaacgtgctgggcaccgagagcagagatggccctggctgggcccagtggctgctgcccccagtggtctggctgctcaatgggctgttggtgctcgtctccttggtgcttctctttgtctacggtgagccagtcacacggccccactcaggccccgccgtgtacttctggaggcatcgcaagcaggctgacctggacctggcccggggagactttgcccaggctgcccagcagctgtggctggccctgcgggcactgggccggcccctgcccacctcccacctggacctggcttgtagcctcctctggaacctcatccgtcacctgctgcagcgtctctgggtgggccgctggctggcaggccgggcagggggcctgcagcaggactgtgctctgcgagtggatgctagcgccagcgcccgagacgcagccctggtctaccataagctgcaccagctgcacaccatggggaagcacacaggcgggcacctcactgccaccaacctggcgctgagtgccctgaacctggcagagtgtgcaggggatgccgtgtctgtggcgacgctggccgagatctatgtggcggctgcattgagagtgaagaccagtctcccacgggccttgcattttctgacacgcttcttcctgagcagtgcccgccaggcctgcctggcacagagtggctcagtgcctcctgccatgcagtggctctgccaccccgtgggccaccgtttcttcgtggatggggactggtccgtgctcagtaccccatgggagagcctgtacagcttggccgggaacccagtggaccccctggcccaggtgactcagctattccgggaacatctcttagagcgagcactgaactgtgtgacccagcccaaccccagccctgggtcagctgatggggacaaggaattctcggatgccctcgggtacctgcagctgctgaacagctgttctgatgctgcgggggctcctgcctacagcttctccatcagttccagcatggccaccaccaccggcgtagacccggtggccaagtggtgggcctctctgacagctgtggtgatccactggctgcggcgggatgaggaggcggctgagcggctgtgcccgctggtggagcacctgccccgggtgctgcaggagtctgagagacccctgcccagggcagctctgcactccttcaaggctgcccgggccctgctgggctgtgccaaggcagagtctggtccagccagcctgaccatctgtgagaaggccagtgggtacctgcaggacagcctggctaccacaccagccagcagctccattgacaaggccgtgcagctgttcctgtgtgacctgcttcttgtggtgcgcaccagcctgtggcggcagcagcagcccccggccccggccccagcagcccagggcaccagcagcaggccccaggcttccgcccttgagctgcgtggcttccaacgggacctgagcagcctgaggcggctggcacagagcttccggcccgccatgcggagggtgttcctacatgaggccacggcccggctgatggcgggggccagccccacacggacacaccagctcctcgaccgcagtctgaggcggcgggcaggccccggtggcaaaggaggcgcggtggcggagctggagccgcggcccacgcggcgggagcacgcggaggccttgctgctggcctcctgctacctgccccccggcttcctgtcggcgcccgggcagcgcgtgggcatgctggctgaggcggcgcgcacactcgagaagcttggcgatcgccggctgctgcacgactgtcagcagatgctcatgcgcctgggcggtgggaccactgtcacttccagctagaccccgtgtccccggcctcagcacccctgtctctagccactttggtcccgtgcagcttctgtcctgcgtcgaagctttgaaggccgaaggcagtgcaagagactctggcctccacagttcgacctgcggctgctgtgtgccttcgcggtggaaggcccgaggggcgcgatcttgaccctaagaccggcggccatgatggtgctgacctctggtggccgatcggggcactgcaggggccgagccattttggggggcccccctccttgctctgcaggcaccttagtggcttttttcctcctgtgtacagggaagagaggggtacatttccctgtgctgacggaagccaacttggctttcccggactgcaagcagggctctgccccagaggcctctctctccgtcgtgggagagagacgtgtacatagtgtaggtcagcgtgcttagcctcctgacctgaggctcctgtgctactttgccttttgcaaactttattttcatagattgagaagttttgtacagagaattaaaaatgaaattatttataatctgggttttgtgtcttcagctgatggatgtgctgactagtgagagtgcttgggccctcccccagcacctagggaaaggcttcccctccccctccggccacaaggtacacaacttttaacttagctcttcccgatgtttgtttgttagtgggaggagtggggagggctggctgtatggcctccagcctacctgttccccctgctcccagggcacatggttgggctgtgtcaacccttagggcctccatggggtcagttgtcccttctcacctcccagctctgtccccatcaggtccctgggtggcacgggaggatggactgacttccaggacctgttgtgtgacaggagctacagcttgggtctccctgcaagaagtctggcacgtctcacctcccccatcccggcccctggtcatctcacagcaaagaagcctcctccctcccgacctgccgccacactggagagggggcacaggggcgggggaggtttcctgttctgtgaaaggccgactccctgactccattcatgcccccccccccagcccctcccttcattcccattccccaacctaaagcctggcccggctcccagctgaatctggtcggaatccacgggctgcagattttccaaaacaatcgttgtatctttattgacttttttttttttttttttctgaatgcaatgactgttttttactcttaaggaaaataaacatcttttagaaacagctcgatacacacaa

SEQ ID NO: 20 - DNA, 4923 bp, Homo sapiens
agcagagctgcggccgggggaacccagtttccgaggaacttttcgccggcgccgggccgcctctgaggccagggcaggacacgaacgcgcggagcggcggcggcgactgagagccggggccgcggcggcgctccctaggaagggccgtacgaggcggcgggcccggcgggcctcccggaggaggcggctgcgccatggacgagccacccttcagcgaggcggctttggagcaggcgctgggcgagccgtgcgatctggacgcggcgctgctgaccgacatcgaagacatgcttcagcttatcaacaaccaagacagtgacttccctggcctatttgacccaccctatgctgggagtggggcagggggcacagaccctgccagccccgataccagctccccaggcagcttgtctccacctcctgccacattgagctcctctcttgaagccttcctgagcgggccgcaggcagcgccctcacccctgtcccctccccagcctgcacccactccattgaagatgtacccgtccatgcccgctttctcccctgggcctggtatcaaggaagagtcagtgccactgagcatcctgcagacccccaccccacagcccctgccaggggccctcctgccacagagcttcccagccccagccccaccgcagttcagctccacccctgtgttaggctaccccagccctccgggaggcttctctacaggaagccctcccgggaacacccagcagccgctgcctggcctgccactggcttccccgccaggggtcccgcccgtctccttgcacacccaggtccagagtgtggtcccccagcagctactgacagtcacagctgcccccacggcagcccctgtaacgaccactgtgacctcgcagatccagcaggtcccggtcctgctgcagccccacttcatcaaggcagactcgctgcttctgacagccatgaagacagacggagccactgtgaaggcggcaggtctcagtcccctggtctctggcaccactgtgcagacagggcctttgccgaccctggtgagtggcggaaccatcttggcaacagtcccactggtcgtagatgcggagaagctgcctatcaaccggctcgcagctggcagcaaggccccggcctctgcccagagccgtggagagaagcgcacagcccacaacgccattgagaagcgctaccgctcctccatcaatgacaaaatcattgagctcaaggatctggtggtgggcactgaggcaaagctgaataaatctgctgtcttgcgcaaggccatcgactacattcgctttctgcaacacagcaaccagaaactcaagcaggagaacctaagtctgcgcactgctgtccacaaaagcaaatctctgaaggatctggtgtcggcctgtggcagtggagggaacacagacgtgctcatggagggcgtgaagactgaggtggaggacacactgaccccacccccctcggatgctggctcacctttccagagcagccccttgtcccttggcagcaggggcagtggcagcggtggcagtggcagtgactcggagcctgacagcccagtctttgaggacagcaaggcaaagccagagcagcggccgtctctgcacagccggggcatgctggaccgctcccgcctggccctgtgcacgctcgtcttcctctgcctgtcctgcaaccccttggcctccttgctgggggcccgggggcttcccagcccctcagataccaccagcgtctaccatagccctgggcgcaacgtgctgggcaccgagagcagagatggccctggctgggcccagtggctgctgcccccagtggtctggctgctcaatgggctgttggtgctcgtctccttggtgcttctctttgtctacggtgagccagtcacacggccccactcaggccccgccgtgtacttctggaggcatcgcaagcaggctgacctggacctggcccggggagactttgcccaggctgcccagcagctgtggctggccctgcgggcactgggccggcccctgcccacctcccacctggacctggcttgtagcctcctctggaacctcatccgtcacctgctgcagcgtctctgggtgggccgctggctggcaggccgggcagggggcctgcagcaggactgtgctctgcgagtggatgctagcgccagcgcccgagacgcagccctggtctaccataagctgcaccagctgcacaccatggggaagcacacaggcgggcacctcactgccaccaacctggcgctgagtgccctgaacctggcagagtgtgcaggggatgccgtgtctgtggcgacgctggccgagatctatgtggcggctgcattgagagtgaagaccagtctcccacgggccttgcattttctgacacgcttcttcctgagcagtgcccgccaggcctgcctggcacagagtggctcagtgcctcctgccatgcagtggctctgccaccccgtgggccaccgtttcttcgtggatggggactggtccgtgctcagtaccccatgggagagcctgtacagcttggccgggaacccagtggaccccctggcccaggtgactcagctattccgggaacatctcttagagcgagcactgaactgtgtgacccagcccaaccccagccctgggtcagctgatggggacaaggaattctcggatgccctcgggtacctgcagctgctgaacagctgttctgatgctgcgggggctcctgcctacagcttctccatcagttccagcatggccaccaccaccggcgtagacccggtggccaagtggtgggcctctctgacagctgtggtgatccactggctgcggcgggatgaggaggcggctgagcggctgtgcccgctggtggagcacctgccccgggtgctgcaggagtctgagagacccctgcccagggcagctctgcactccttcaaggctgcccgggccctgctgggctgtgccaaggcagagtctggtccagccagcctgaccatctgtgagaaggccagtgggtacctgcaggacagcctggctaccacaccagccagcagctccattgacaaggccgtgcagctgttcctgtgtgacctgcttcttgtggtgcgcaccagcctgtggcggcagcagcagcccccggccccggccccagcagcccagggcaccagcagcaggccccaggcttccgcccttgagctgcgtggcttccaacgggacctgagcagcctgaggcggctggcacagagcttccggcccgccatgcggagggtgttcctacatgaggccacggcccggctgatggcgggggccagccccacacggacacaccagctcctcgaccgcagtctgaggcggcgggcaggccccggtggcaaaggaggcgcggtggcggagctggagccgcggcccacgcggcgggagcacgcggaggccttgctgctggcctcctgctacctgccccccggcttcctgtcggcgcccgggcsagcgcgtgggcatgctggctgaggcggcgcgcacactcgagaagcttggcgatcgccggctgctgcacgactgtcagcagatgctcatgcgcctgggcggtgggaccactgtcacttccagctagaccccgtgtccccggcctcagcacccctgtctctagccactttggtcccgtgcagcttctgtcctgcgtcgaagctttgaaggccgaaggcagtgcaagagactctggcctccacagttcgacctgcggctgctgtgtgccttcgcggtggaaggcccgaggggcgcgatcttgaccctaagaccggcggccatgatggtgctgacctctggtggccgatcggggcactgcaggggccgagccattttggggggcccccctccttgctctgcaggcaccttagtggcttttttcctcctgtgtacagggaagagaggggtacatttccctgtgctgacggaagccaacttggctttcccggactgcaagcagggctctgccccagaggcctctctctccgtcgtgggagagagacgtgtacatagtgtaggtcagcgtgcttagcctcctgacctgaggctcctgtgctactttgccttttgcaaactttattttcatagattgagaagttttgtacagagaattaaaaatgaaattatttataatctgggttttgtgtcttcagctgatggatgtgctgactagtgagagtgcttgggccctcccccagcacctagggaaaggcttcccctccccctccggccacaaggtacacaacttttaacttagctcttcccgatgtttgtttgttagtgggaggagtggggagggctggctgtatggcctccagcctacctgttccccctgctcccagggcacatggttgggctgtgtcaacccttagggcctccatggggtcagttgtcccttctcacctcccagctctgtccccatcaggtccctgggtggcacgggaggatggactgacttccaggacctgttgtgtgacaggagctacagcttgggtctccctgcaagaagtctggcacgtctcacctcccccatcccggcccctggtcatctcacagcaaagaagcctcctccctcccgacctgccgccacactggagagggggcacaggggcgggggaggtttcctgttctgtgaaaggccgactccctgactccattcatgcccccccccccagcccctcccttcattcccattccccaacctaaagcctggcccggctcccagctgaatctggtcggaatccacgggctgcagattttccaaaacaatcgttgtatctttattgacttttttttttttttttttctgaatgcaatgactgttttttactcttaaggaaaataaacatcttttagaaacaaaaaaaaaaaa

SEQ ID NO: 21 - DNA, 4754 bp, Homo sapiens
aaaaatccgccgcgccttgacaggtgaagtcggcgcggggaggggtagggccaacggcctggacgccccaagggcgggcgcagatcgcggagccatggattgcactttcgaagacatgcttcagcttatcaacaaccaagacagtgacttccctggcctatttgacccaccctatgctgggagtggggcagggggcacagaccctgccagccccgataccagctccccaggcagcttgtctccacctcctgccacattgagctcctctcttgaagccttcctgagcgggccgcaggcagcgccctcacccctgtcccctccccagcctgcacccactccattgaagatgtacccgtccatgcccgctttctcccctgggcctggtatcaaggaagagtcagtgccactgagcatcctgcagacccccaccccacagcccctgccaggggccctcctgccacagagcttcccagccccagccccaccgcagttcagctccacccctgtgttaggctaccccagccctccgggaggcttctctacaggaagccctcccgggaacacccagcagccgctgcctggcctgccactggcttccccgccaggggtcccgcccgtctccttgcacacccaggtccagagtgtggtcccccagcagctactgacagtcacagctgcccccacggcagcccctgtaacgaccactgtgacctcgcagatccagcaggtcccggtcctgctgcagccccacttcatcaaggcagactcgctgcttctgacagccatgaagacagacggagccactgtgaaggcggcaggtctcagtcccctggtctctggcaccactgtgcagacagggcctttgccgaccctggtgagtggcggaaccatcttggcaacagtcccactggtcgtagatgcggagaagctgcctatcaaccggctcgcagctggcagcaaggccccggcctctgcccagagccgtggagagaagcgcacagcccacaacgccattgagaagcgctaccgctcctccatcaatgacaaaatcattgagctcaaggatctggtggtgggcactgaggcaaagctgaataaatctgctgtcttgcgcaaggccatcgactacattcgctttctgcaacacagcaaccagaaactcaagcaggagaacctaagtctgcgcactgctgtccacaaaagcaaatctctgaaggatctggtgtcggcctgtggcagtggagggaacacagacgtgctcatggagggcgtgaagactgaggtggaggacacactgaccccacccccctcggatgctggctcacctttccagagcagccccttgtcccttggcagcaggggcagtggcagcggtggcagtggcagtgactcggagcctgacagcccagtctttgaggacagcaaggcaaagccagagcagcggccgtctctgcacagccggggcatgctggaccgctcccgcctggccctgtgcacgctcgtcttcctctgcctgtcctgcaaccccttggcctccttgctgggggcccgggggcttcccagcccctcagataccaccagcgtctaccatagccctgggcgcaacgtgctgggcaccgagagcagagatggccctggctgggcccagtggctgctgcccccagtggtctggctgctcaatgggctgttggtgctcgtctccttggtgcttctctttgtctacggtgagccagtcacacggccccactcaggccccgccgtgtacttctggaggcatcgcaagcaggctgacctggacctggcccggggagactttgcccaggctgcccagcagctgtggctggccctgcgggcactgggccggcccctgcccacctcccacctggacctggcttgtagcctcctctggaacctcatccgtcacctgctgcagcgtctctgggtgggccgctggctggcaggccgggcagggggcctgcagcaggactgtgctctgcgagtggatgctagcgccagcgcccgagacgcagccctggtctaccataagctgcaccagctgcacaccatggggaagcacacaggcgggcacctcactgccaccaacctggcgctgagtgccctgaacctggcagagtgtgcaggggatgccgtgtctgtggcgacgctggccgagatctatgtggcggctgcattgagagtgaagaccagtctcccacgggccttgcattttctgacacgcttcttcctgagcagtgcccgccaggcctgcctggcacagagtggctcagtgcctcctgccatgcagtggctctgccaccccgtgggccaccgtttcttcgtggatggggactggtccgtgctcagtaccccatgggagagcctgtacagcttggccgggaacccagtggaccccctggcccaggtgactcagctattccgggaacatctcttagagcgagcactgaactgtgtgacccagcccaaccccagccctgggtcagctgatggggacaaggaattctcggatgccctcgggtacctgcagctgctgaacagctgttctgatgctgcgggggctcctgcctacagcttctccatcagttccagcatggccaccaccaccggcgtagacccggtggccaagtggtgggcctctctgacagctgtggtgatccactggctgcggcgggatgaggaggcggctgagcggctgtgcccgctggtggagcacctgccccgggtgctgcaggagtctgagagacccctgcccagggcagctctgcactccttcaaggctgcccgggccctgctgggctgtgccaaggcagagtctggtccagccagcctgaccatctgtgagaaggccagtgggtacctgcaggacagcctggctaccacaccagccagcagctccattgacaaggccgtgcagctgttcctgtgtgacctgcttcttgtggtgcgcaccagcctgtggcggcagcagcagcccccggccccggccccagcagcccagggcaccagcagcaggccccaggcttccgcccttgagctgcgtggcttccaacgggacctgagcagcctgaggcggctggcacagagcttccggcccgccatgcggagggtgttcctacatgaggccacggcccggctgatggcgggggccagccccacacggacacaccagctcctcgaccgcagtctgaggcggcgggcaggccccggtggcaaaggaggcgcggtggcggagctggagccgcggcccacgcggcgggagcacgcggaggccttgctgctggcctcctgctacctgccccccggcttcctgtcggcgcccgggcagcgcgtgggcatgctggctgaggcggcgcgcacactcgagaagcttggcgatcgccggctgctgcacgactgtcagcagatgctcatgcgcctgggcggtgggaccactgtcacttccagctagaccccgtgtccccggcctcagcacccctgtctctagccactttggtcccgtgcagcttctgtcctgcgtcgaagctttgaaggccgaaggcagtgcaagagactctggcctccacagttcgacctgcggctgctgtgtgccttcgcggtggaaggcccgaggggcgcgatcttgaccctaagaccggcggccatgatggtgctgacctctggtggccgatcggggcactgcaggggccgagccattttggggggcccccctccttgctctgcaggcaccttagtggcttttttcctcctgtgtacagggaagagaggggtacatttccctgtgctgacggaagccaacttggctttcccggactgcaagcagggctctgccccagaggcctctctctccgtcgtgggagagagacgtgtacatagtgtaggtcagcgtgcttagcctcctgacctgaggctcctgtgctactttgccttttgcaaactttattttcatagattgagaagttttgtacagagaattaaaaatgaaattatttataatctgggttttgtgtcttcagctgatggatgtgctgactagtgagagtgcttgggccctcccccagcacctagggaaaggcttcccctccccctccggccacaaggtacacaacttttaacttagctcttcccgatgtttgtttgttagtgggaggagtggggagggctggctgtatggcctccagcctacctgttccccctgctcccagggcacatggttgggctgtgtcaacccttagggcctccatggggtcagttgtcccttctcacctcccagctctgtccccatcaggtccctgggtggcacgggaggatggactgacttccaggacctgttgtgtgacaggagctacagcttgggtctccctgcaagaagtctggcacgtctcacctcccccatcccggcccctggtcatctcacagcaaagaagcctcctccctcccgacctgccgccacactggagagggggcacaggggcgggggaggtttcctgttctgtgaaaggccgactccctgactccattcatgcccccccccccagcccctcccttcattcccattccccaacctaaagcctggcccggctcccagctgaatctggtcggaatccacgggctgcagattttccaaaacaatcgttgtatctttattgacttttttttttttttttttctgaatgcaatgactgttttttactcttaaggaaaataaacatcttttagaaacagctcgatacacacaa

SEQ ID NO: 22 - DNA, 8779 bp, Homo sapiens
gagaaggaggaggacagcgccgaggaggaagaggttgatggcggcggcggagctccgagagacctcggctgggcaggggccggccgtggcgggccggggactgcgcctctagagccgcgagttctcgggaattcgccgcagcggacgcgctcggcgaatttgtgctcttgtgccctcctccgggcttgggcccaggcccggcccctcgcacttgcccttaccttttctatcgagtccgcatccctctccagccactgcgacccggcgaagagaaaaaggaacttcccccaccccctcgggtgccgtcggagccccccagcccacccctgggtgcggcgcggggaccccgggccgaagaagagatttcctgaggattctggttttcctcgcttgtatctccgaaagaattaaaaatggccgagaatgtggtggaaccggggccgccttcagccaagcggcctaaactctcatctccggccctctcggcgtccgccagcgatggcacagattttggctctctatttgacttggagcacgacttaccagatgaattaatcaactctacagaattgggactaaccaatggtggtgatattaatcagcttcagacaagtcttggcatggtacaagatgcagcttctaaacataaacagctgtcagaattgctgcgatctggtagttcccctaacctcaatatgggagttggtggcccaggtcaagtcatggccagccaggcccaacagagcagtcctggattaggtttgataaatagcatggtcaaaagcccaatgacacaggcaggcttgacttctcccaacatggggatgggcactagtggaccaaatcagggtcctacgcagtcaacaggtatgatgaacagtccagtaaatcagcctgccatgggaatgaacacagggatgaatgcgggcatgaatcctggaatgttggctgcaggcaatggacaagggataatgcctaatcaagtcatgaacggttcaattggagcaggccgagggcgacagaatatgcagtacccaaacccaggcatgggaagtgctggcaacttactgactgagcctcttcagcagggctctccccagatgggaggacaaacaggattgagaggcccccagcctcttaagatgggaatgatgaacaaccccaatccttatggttcaccatatactcagaatcctggacagcagattggagccagtggccttggtctccagattcagacaaaaactgtactatcaaataacttatctccatttgctatggacaaaaaggcagttcctggtggaggaatgcccaacatgggtcaacagccagccccgcaggtccagcagccaggcctggtgactccagttgcccaagggatgggttctggagcacatacagctgatccagagaagcgcaagctcatccagcagcagcttgttctccttttgcatgctcacaagtgccagcgccgggaacaggccaatggggaagtgaggcagtgcaaccttccccactgtcgcacaatgaagaatgtcctaaaccacatgacacactgccagtcaggcaagtcttgccaagtggcacactgtgcatcttctcgacaaatcatttcacactggaagaattgtacaagacatgattgtcctgtgtgtctccccctcaaaaatgctggtgataagagaaatcaacagccaattttgactggagcacccgttggacttggaaatcctagctctctaggggtgggtcaacagtctgcccccaacctaagcactgttagtcagattgatcccagctccatagaaagagcctatgcagctcttggactaccctatcaagtaaatcagatgccgacacaaccccaggtgcaagcaaagaaccagcagaatcagcagcctgggcagtctccccaaggcatgcggcccatgagcaacatgagtgctagtcctatgggagtaaatggaggtgtaggagttcaaacgccgagtcttctttctgactcaatgttgcattcagccataaattctcaaaacccaatgatgagtgaaaatgccagtgtgccctccctgggtcctatgccaacagcagctcaaccatccactactggaattcggaaacagtggcacgaagatattactcaggatcttcgaaatcatcttgttcacaaactcgtccaagccatatttcctacgccggatcctgctgctttaaaagacagacggatggaaaacctagttgcatatgctcggaaagttgaaggggacatgtatgaatctgcaaacaatcgagcggaatactaccaccttctagctgagaaaatctataagatccagaaagaactagaagaaaaacgaaggaccagactacagaagcagaacatgctaccaaatgctgcaggcatggttccagtttccatgaatccagggcctaacatgggacagccgcaaccaggaatgacttctaatggccctctacctgacccaagtatgatccgtggcagtgtgccaaaccagatgatgcctcgaataactccacaatctggtttgaatcaatttggccagatgagcatggcccagccccctattgtaccccggcaaacccctcctcttcagcaccatggacagttggctcaacctggagctctcaacccgcctatgggctatgggcctcgtatgcaacagccttccaaccagggccagttccttcctcagactcagttcccatcacagggaatgaatgtaacaaatatccctttggctccgtccagcggtcaagctccagtgtctcaagcacaaatgtctagttcttcctgcccggtgaactctcctataatgcctccagggtctcaggggagccacattcactgtccccagcttcctcaaccagctcttcatcagaattcaccctcgcctgtacctagtcgtacccccacccctcaccatactcccccaagcataggggctcagcagccaccagcaacaacaattccagcccctgttcctacacctcctgccatgccacctgggccacagtcccaggctctacatccccctccaaggcagacacctacaccaccaacaacacaacttccccaacaagtgcagccttcacttcctgctgcaccttctgctgaccagccccagcagcagcctcgctcacagcagagcacagcagcgtctgttcctaccccaacagcaccgctgcttcctccgcagcctgcaactccactttcccagccagctgtaagcattgaaggacaggtatcaaatcctccatctactagtagcacagaagtgaattctcaggccattgctgagaagcagccttcccaggaagtgaagatggaggccaaaatggaagtggatcaaccagaaccagcagatactcagccggaggatatttcagagtctaaagtggaagactgtaaaatggaatctaccgaaacagaagagagaagcactgagttaaaaactgaaataaaagaggaggaagaccagccaagtacttcagctacccagtcatctccggctccaggacagtcaaagaaaaagattttcaaaccagaagaactacgacaggcactgatgccaactttggaggcactttaccgtcaggatccagaatcccttccctttcgtcaacctgtggaccctcagcttttaggaatccctgattactttgatattgtgaagagccccatggatctttctaccattaagaggaagttagacactggacagtatcaggagccctggcagtatgtcgatgatatttggcttatgttcaataatgcctggttatataaccggaaaacatcacgggtatacaaatactgctccaagctctctgaggtctttgaacaagaaattgacccagtgatgcaaagccttggatactgttgtggcagaaagttggagttctctccacagacactgtgttgctacggcaaacagttgtgcacaatacctcgtgatgccacttattacagttaccagaacaggtatcatttctgtgagaagtgtttcaatgagatccaaggggagagcgtttctttgggggatgacccttcccagcctcaaactacaataaataaagaacaattttccaagagaaaaaatgacacactggatcctgaactgtttgttgaatgtacagagtgcggaagaaagatgcatcagatctgtgtccttcaccatgagatcatctggcctgctggattcgtctgtgatggctgtttaaagaaaagtgcacgaactaggaaagaaaataagttttctgctaaaaggttgccatctaccagacttggcacctttctagagaatcgtgtgaatgactttctgaggcgacagaatcaccctgagtcaggagaggtcactgttagagtagttcatgcttctgacaaaaccgtggaagtaaaaccaggcatgaaagcaaggtttgtggacagtggagagatggcagaatcctttccataccgaaccaaagccctctttgcctttgaagaaattgatggtgttgacctgtgcttctttggcatgcatgttcaagagtatggctctgactgccctccacccaaccagaggagagtatacatatcttacctcgatagtgttcatttcttccgtcctaaatgcttgaggactgcagtctatcatgaaatcctaattggatatttagaatatgtcaagaaattaggttacacaacagggcatatttgggcatgtccaccaagtgagggagatgattatatcttccattgccatcctcctgaccagaagatacccaagcccaagcgactgcaggaatggtacaaaaaaatgcttgacaaggctgtatcagagcgtattgtccatgactacaaggatatttttaaacaagctactgaagatagattaacaagtgcaaaggaattgccttatttcgagggtgatttctggcccaatgttctggaagaaagcattaaggaactggaacaggaggaagaagagagaaaacgagaggaaaacaccagcaatgaaagcacagatgtgaccaagggagacagcaaaaatgctaaaaagaagaataataagaaaaccagcaaaaataagagcagcctgagtaggggcaacaagaagaaacccgggatgcccaatgtatctaacgacctctcacagaaactatatgccaccatggagaagcataaagaggtcttctttgtgatccgcctcattgctggccctgctgccaactccctgcctcccattgttgatcctgatcctctcatcccctgcgatctgatggatggtcgggatgcgtttctcacgctggcaagggacaagcacctggagttctcttcactccgaagagcccagtggtccaccatgtgcatgctggtggagctgcacacgcagagccaggaccgctttgtctacacctgcaatgaatgcaagcaccatgtggagacacgctggcactgtactgtctgtgaggattatgacttgtgtatcacctgctataacactaaaaaccatgaccacaaaatggagaaactaggccttggcttagatgatgagagcaacaaccagcaggctgcagccacccagagcccaggcgattctcgccgcctgagtatccagcgctgcatccagtctctggtccatgcttgccagtgtcggaatgccaattgctcactgccatcctgccagaagatgaagcgggttgtgcagcataccaagggttgcaaacggaaaaccaatggcgggtgccccatctgcaagcagctcattgccctctgctgctaccatgccaagcactgccaggagaacaaatgcccggtgccgttctgcctaaacatcaagcagaagctccggcagcaacagctgcagcaccgactacagcaggcccaaatgcttcgcaggaggatggccagcatgcagcggactggtgtggttgggcagcaacagggcctcccttcccccactcctgccactccaacgacaccaactggccaacagccaaccaccccgcagacgccccagcccacttctcagcctcagcctacccctcccaatagcatgccaccctacttgcccaggactcaagctgctggccctgtgtcccagggtaaggcagcaggccaggtgacccctccaacccctcctcagactgctcagccaccccttccagggcccccacctgcagcagtggaaatggcaatgcagattcagagagcagcggagacgcagcgccagatggcccacgtgcaaatttttcaaaggccaatccaacaccagatgcccccgatgactcccatggcccccatgggtatgaacccacctcccatgaccagaggtcccagtgggcatttggagccagggatgggaccgacagggatgcagcaacagccaccctggagccaaggaggattgcctcagccccagcaactacagtctgggatgccaaggccagccatgatgtcagtggcccagcatggtcaacctttgaacatggctccacaaccaggattgggccaggtaggtatcagcccactcaaaccaggcactgtgtctcaacaagccttacaaaaccttttgcggactctcaggtctcccagctctcccctgcagcagcaacaggtgcttagtatccttcacgccaacccccagctgttggctgcattcatcaagcagcgggctgccaagtatgccaactctaatccacaacccatccctgggcagcctggcatgccccaggggcagccagggctacagccacctaccatgccaggtcagcagggggtccactccaatccagccatgcagaacatgaatccaatgcaggcgggcgttcagagggctggcctgccccagcagcaaccacagcagcaactccagccacccatgggagggatgagcccccaggctcagcagatgaacatgaaccacaacaccatgccttcacaattccgagacatcttgagacgacagcaaatgatgcaacagcagcagcaacagggagcagggccaggaataggccctggaatggccaaccataaccagttccagcaaccccaaggagttggctacccaccacagcagcagcagcggatgcagcatcacatgcaacagatgcaacaaggaaatatgggacagataggccagcttccccaggccttgggagcagaggcaggtgccagtctacaggcctatcagcagcgactccttcagcaacagatggggtcccctgttcagcccaaccccatgagcccccagcagcatatgctcccaaatcaggcccagtccccacacctacaaggccagcagatccctaattctctctccaatcaagtgcgctctccccagcctgtcccttctccacggccacagtcccagcccccccactccagtccttccccaaggatgcagcctcagccttctccacaccacgtttccccacagacaagttccccacatcctggactggtagctgcccaggccaaccccatggaacaagggcattttgccagcccggaccagaattcaatgctttctcagcttgctagcaatccaggcatggcaaacctccatggtgcaagcgccacggacctgggactcagcaccgataactcagacttgaattcaaacctctcacagagtacactagacatacactagagacaccttgtagtattttgggagcaaaaaaattattttctcttaacaagactttttgtactgaaaacaatttttttgaatctttcgtagcctaaaagacaattttccttggaacacataagaactgtgcagtagccgtttgtggtttaaagcaaacatgcaagatgaacctgagggatgatagaatacaaagaatatatttttgttatggctggttaccaccagcctttcttcccctttgtgtgtgtggttcaagtgtgcactgggaggaggctgaggcctgtgaagccaaacaatatgctcctgccttgcacctccaataggttttattattttttttaaattaatgaacatatgtaatattaatagttattatttactggtgcagatggttgacatttttccctattttcctcactttatggaagagttaaaacatttctaaaccagaggacaaaaggggttaatgttactttaaaattacattctatatatatataaatatatataaatatatattaaaataccagttttttttctctgggtgcaaagatgttcattcttttaaaaaatgtttaaaaaaaaaaaaaaactgcctttcttcccctcaagtcaacttttgtgctccagaaaattttctattctgtaagtctgagcgtaaaacttcaagtattaaaataatttgtacatgtagagagaaaaatgactttttcaaaaatatacaggggcagctgccaaattgatgtattatatattgtggtttctgtttcttgaaagaatttttttcgttatttttacatctaacaaagtaaaaaaattaaaaagagggtaagaaacgattccggtgggatgattttaacatgcaaaatgtccctgggggtttcttctttgcttgctttcttcctccttaccctaccccccactcacacacacacacacacacacacacacacacacacacacacactttctataaaacttgaaaatagcaaaaaccctcaactgttgtaaatcatgcaattaaagttgattacttataaatatgaactttggatcactgtatagactgttaaatttgatttcttattacctattgttaaataaactgtgtgagacagaca

SEQ ID NO: 23 - DNA, 8701 bp, Homo sapiens
gagaaggaggaggacagcgccgaggaggaagaggttgatggcggcggcggagctccgagagacctcggctgggcaggggccggccgtggcgggccggggactgcgcctctagagccgcgagttctcgggaattcgccgcagcggacgcgctcggcgaatttgtgctcttgtgccctcctccgggcttgggcccaggcccggcccctcgcacttgcccttaccttttctatcgagtccgcatccctctccagccactgcgacccggcgaagagaaaaaggaacttcccccaccccctcgggtgccgtcggagccccccagcccacccctgggtgcggcgcggggaccccgggccgaagaagagatttcctgaggattctggttttcctcgcttgtatctccgaaagaattaaaaatggccgagaatgtggtggaaccggggccgccttcagccaagcggcctaaactctcatctccggccctctcggcgtccgccagcgatggcacagattttggctctctatttgacttggagcacgacttaccagatgaattaatcaactctacagaattgggactaaccaatggtggtgatattaatcagcttcagacaagtcttggcatggtacaagatgcagcttctaaacataaacagctgtcagaattgctgcgatctggtagttcccctaacctcaatatgggagttggtggcccaggtcaagtcatggccagccaggcccaacagagcagtcctggattaggtttgataaatagcatggtcaaaagcccaatgacacaggcaggcttgacttctcccaacatggggatgggcactagtggaccaaatcagggtcctacgcagtcaacaggtatgatgaacagtccagtaaatcagcctgccatgggaatgaacacagggatgaatgcgggcatgaatcctggaatgttggctgcaggcaatggacaagggataatgcctaatcaagtcatgaacggttcaattggagcaggccgagggcgacagaatatgcagtacccaaacccaggcatgggaagtgctggcaacttactgactgagcctcttcagcagggctctccccagatgggaggacaaacaggattgagaggcccccagcctcttaagatgggaatgatgaacaaccccaatccttatggttcaccatatactcagaatcctggacagcagattggagccagtggccttggtctccagattcagacaaaaactgtactatcaaataacttatctccatttgctatggacaaaaaggcagttcctggtggaggaatgcccaacatgggtcaacagccagccccgcaggtccagcagccaggcctggtgactccagttgcccaagggatgggttctggagcacatacagctgatccagagaagcgcaagctcatccagcagcagcttgttctccttttgcatgctcacaagtgccagcgccgggaacaggccaatggggaagtgaggcagtgcaaccttccccactgtcgcacaatgaagaatgtcctaaaccacatgacacactgccagtcaggcaagtcttgccaagtggcacactgtgcatcttctcgacaaatcatttcacactggaagaattgtacaagacatgattgtcctgtgtgtctccccctcaaaaatgctggtgataagagaaatcaacagccaattttgactggagcacccgttggacttggaaatcctagctctctaggggtgggtcaacagtctgcccccaacctaagcactgttagtcagattgatcccagctccatagaaagagcctatgcagctcttggactaccctatcaagtaaatcagatgccgacacaaccccaggtgcaagcaaagaaccagcagaatcagcagcctgggcagtctccccaaggcatgcggcccatgagcaacatgagtgctagtcctatgggagtaaatggaggtgtaggagttcaaacgccgagtcttctttctgactcaatgttgcattcagccataaattctcaaaacccaatgatgagtgaaaatgccagtgtgccctccctgggtcctatgccaacagcagctcaaccatccactactggaattcggaaacagtggcacgaagatattactcaggatcttcgaaatcatcttgttcacaaactcgtccaagccatatttcctacgccggatcctgctgctttaaaagacagacggatggaaaacctagttgcatatgctcggaaagttgaaggggacatgtatgaatctgcaaacaatcgagcggaatactaccaccttctagctgagaaaatctataagatccagaaagaactagaagaaaaacgaaggaccagactacagaagcagaacatgctaccaaatgctgcaggcatggttccagtttccatgaatccagggcctaacatgggacagccgcaaccaggaatgacttctagtttgaatcaatttggccagatgagcatggcccagccccctattgtaccccggcaaacccctcctcttcagcaccatggacagttggctcaacctggagctctcaacccgcctatgggctatgggcctcgtatgcaacagccttccaaccagggccagttccttcctcagactcagttcccatcacagggaatgaatgtaacaaatatccctttggctccgtccagcggtcaagctccagtgtctcaagcacaaatgtctagttcttcctgcccggtgaactctcctataatgcctccagggtctcaggggagccacattcactgtccccagcttcctcaaccagctcttcatcagaattcaccctcgcctgtacctagtcgtacccccacccctcaccatactcccccaagcataggggctcagcagccaccagcaacaacaattccagcccctgttcctacacctcctgccatgccacctgggccacagtcccaggctctacatccccctccaaggcagacacctacaccaccaacaacacaacttccccaacaagtgcagccttcacttcctgctgcaccttctgctgaccagccccagcagcagcctcgctcacagcagagcacagcagcgtctgttcctaccccaacagcaccgctgcttcctccgcagcctgcaactccactttcccagccagctgtaagcattgaaggacaggtatcaaatcctccatctactagtagcacagaagtgaattctcaggccattgctgagaagcagccttcccaggaagtgaagatggaggccaaaatggaagtggatcaaccagaaccagcagatactcagccggaggatatttcagagtctaaagtggaagactgtaaaatggaatctaccgaaacagaagagagaagcactgagttaaaaactgaaataaaagaggaggaagaccagccaagtacttcagctacccagtcatctccggctccaggacagtcaaagaaaaagattttcaaaccagaagaactacgacaggcactgatgccaactttggaggcactttaccgtcaggatccagaatcccttccctttcgtcaacctgtggaccctcagcttttaggaatccctgattactttgatattgtgaagagccccatggatctttctaccattaagaggaagttagacactggacagtatcaggagccctggcagtatgtcgatgatatttggcttatgttcaataatgcctggttatataaccggaaaacatcacgggtatacaaatactgctccaagctctctgaggtctttgaacaagaaattgacccagtgatgcaaagccttggatactgttgtggcagaaagttggagttctctccacagacactgtgttgctacggcaaacagttgtgcacaatacctcgtgatgccacttattacagttaccagaacaggtatcatttctgtgagaagtgtttcaatgagatccaaggggagagcgtttctttgggggatgacccttcccagcctcaaactacaataaataaagaacaattttccaagagaaaaaatgacacactggatcctgaactgtttgttgaatgtacagagtgcggaagaaagatgcatcagatctgtgtccttcaccatgagatcatctggcctgctggattcgtctgtgatggctgtttaaagaaaagtgcacgaactaggaaagaaaataagttttctgctaaaaggttgccatctaccagacttggcacctttctagagaatcgtgtgaatgactttctgaggcgacagaatcaccctgagtcaggagaggtcactgttagagtagttcatgcttctgacaaaaccgtggaagtaaaaccaggcatgaaagcaaggtttgtggacagtggagagatggcagaatcctttccataccgaaccaaagccctctttgcctttgaagaaattgatggtgttgacctgtgcttctttggcatgcatgttcaagagtatggctctgactgccctccacccaaccagaggagagtatacatatcttacctcgatagtgttcatttcttccgtcctaaatgcttgaggactgcagtctatcatgaaatcctaattggatatttagaatatgtcaagaaattaggttacacaacagggcatatttgggcatgtccaccaagtgagggagatgattatatcttccattgccatcctcctgaccagaagatacccaagcccaagcgactgcaggaatggtacaaaaaaatgcttgacaaggctgtatcagagcgtattgtccatgactacaaggatatttttaaacaagctactgaagatagattaacaagtgcaaaggaattgccttatttcgagggtgatttctggcccaatgttctggaagaaagcattaaggaactggaacaggaggaagaagagagaaaacgagaggaaaacaccagcaatgaaagcacagatgtgaccaagggagacagcaaaaatgctaaaaagaagaataataagaaaaccagcaaaaataagagcagcctgagtaggggcaacaagaagaaacccgggatgcccaatgtatctaacgacctctcacagaaactatatgccaccatggagaagcataaagaggtcttctttgtgatccgcctcattgctggccctgctgccaactccctgcctcccattgttgatcctgatcctctcatcccctgcgatctgatggatggtcgggatgcgtttctcacgctggcaagggacaagcacctggagttctcttcactccgaagagcccagtggtccaccatgtgcatgctggtggagctgcacacgcagagccaggaccgctttgtctacacctgcaatgaatgcaagcaccatgtggagacacgctggcactgtactgtctgtgaggattatgacttgtgtatcacctgctataacactaaaaaccatgaccacaaaatggagaaactaggccttggcttagatgatgagagcaacaaccagcaggctgcagccacccagagcccaggcgattctcgccgcctgagtatccagcgctgcatccagtctctggtccatgcttgccagtgtcggaatgccaattgctcactgccatcctgccagaagatgaagcgggttgtgcagcataccaagggttgcaaacggaaaaccaatggcgggtgccccatctgcaagcagctcattgccctctgctgctaccatgccaagcactgccaggagaacaaatgcccggtgccgttctgcctaaacatcaagcagaagctccggcagcaacagctgcagcaccgactacagcaggcccaaatgcttcgcaggaggatggccagcatgcagcggactggtgtggttgggcagcaacagggcctcccttcccccactcctgccactccaacgacaccaactggccaacagccaaccaccccgcagacgccccagcccacttctcagcctcagcctacccctcccaatagcatgccaccctacttgcccaggactcaagctgctggccctgtgtcccagggtaaggcagcaggccaggtgacccctccaacccctcctcagactgctcagccaccccttccagggcccccacctgcagcagtggaaatggcaatgcagattcagagagcagcggagacgcagcgccagatggcccacgtgcaaatttttcaaaggccaatccaacaccagatgcccccgatgactcccatggcccccatgggtatgaacccacctcccatgaccagaggtcccagtgggcatttggagccagggatgggaccgacagggatgcagcaacagccaccctggagccaaggaggattgcctcagccccagcaactacagtctgggatgccaaggccagccatgatgtcagtggcccagcatggtcaacctttgaacatggctccacaaccaggattgggccaggtaggtatcagcccactcaaaccaggcactgtgtctcaacaagccttacaaaaccttttgcggactctcaggtctcccagctctcccctgcagcagcaacaggtgcttagtatccttcacgccaacccccagctgttggctgcattcatcaagcagcgggctgccaagtatgccaactctaatccacaacccatccctgggcagcctggcatgccccaggggcagccagggctacagccacctaccatgccaggtcagcagggggtccactccaatccagccatgcagaacatgaatccaatgcaggcgggcgttcagagggctggcctgccccagcagcaaccacagcagcaactccagccacccatgggagggatgagcccccaggctcagcagatgaacatgaaccacaacaccatgccttcacaattccgagacatcttgagacgacagcaaatgatgcaacagcagcagcaacagggagcagggccaggaataggccctggaatggccaaccataaccagttccagcaaccccaaggagttggctacccaccacagcagcagcagcggatgcagcatcacatgcaacagatgcaacaaggaaatatgggacagataggccagcttccccaggccttgggagcagaggcaggtgccagtctacaggcctatcagcagcgactccttcagcaacagatggggtcccctgttcagcccaaccccatgagcccccagcagcatatgctcccaaatcaggcccagtccccacacctacaaggccagcagatccctaattctctctccaatcaagtgcgctctccccagcctgtcccttctccacggccacagtcccagcccccccactccagtccttccccaaggatgcagcctcagccttctccacaccacgtttccccacagacaagttccccacatcctggactggtagctgcccaggccaaccccatggaacaagggcattttgccagcccggaccagaattcaatgctttctcagcttgctagcaatccaggcatggcaaacctccatggtgcaagcgccacggacctgggactcagcaccgataactcagacttgaattcaaacctctcacagagtacactagacatacactagagacaccttgtagtattttgggagcaaaaaaattattttctcttaacaagactttttgtactgaaaacaatttttttgaatctttcgtagcctaaaagacaattttccttggaacacataagaactgtgcagtagccgtttgtggtttaaagcaaacatgcaagatgaacctgagggatgatagaatacaaagaatatatttttgttatggctggttaccaccagcctttcttcccctttgtgtgtgtggttcaagtgtgcactgggaggaggctgaggcctgtgaagccaaacaatatgctcctgccttgcacctccaataggttttattattttttttaaattaatgaacatatgtaatattaatagttattatttactggtgcagatggttgacatttttccctattttcctcactttatggaagagttaaaacatttctaaaccagaggacaaaaggggttaatgttactttaaaattacattctatatatatataaatatatataaatatatattaaaataccagttttttttctctgggtgcaaagatgttcattcttttaaaaaatgtttaaaaaaaaaaaaaaactgcctttcttcccctcaagtcaacttttgtgctccagaaaattttctattctgtaagtctgagcgtaaaacttcaagtattaaaataatttgtacatgtagagagaaaaatgactttttcaaaaatatacaggggcagctgccaaattgatgtattatatattgtggtttctgtttcttgaaagaatttttttcgttatttttacatctaacaaagtaaaaaaattaaaaagagggtaagaaacgattccggtgggatgattttaacatgcaaaatgtccctgggggtttcttctttgcttgctttcttcctccttaccctaccccccactcacacacacacacacacacacacacacacacacacacacacactttctataaaacttgaaaatagcaaaaaccctcaactgttgtaaatcatgcaattaaagttgattacttataaatatgaactttggatcactgtatagactgttaaatttgatttcttattacctattgttaaataaactgtgtgagacagaca

SEQ ID NO: 24 - DNA, 5867 bp, Homo sapiens
actgccctcgcttcctgtgcctcttcaggtcatcgcttgctctcgttcccaggctttggcttctagtggacgagaatcaccgagtctgcggggctggatgctgaccgcccggaccagcacctaggcgggcgggagctgtgcggcccagggttcgcgcgggccgggtagaggctcgagctgggacccccgagcgtgaaccccggagccggcggcgctggggccagaggggccgggagccccagggaggcggatctgggccccgagaaggacacccgcctggatttgccccgtaggcccggcccgggcccctcgggagcagaacagccttggtgaggtggacaggaggggacctcgcgagcagacgcgcgcgccagcgacagcagcccgccccggcctctcgggagccgtggggcagaggctgcggagccccaggagggggccagtgtcattcaaagatgtggctgtggatttcacccaggaggagtggcggcaactggaccctgatgagaagataacatacggggatgtgatgttggagaactacagccatctagtttccttggcttatgaggtggcaacatcttgtacttcggagattctgaagccgagcaacttgcccaagtccttcttcttttcccattaacaagatatgatatcaccaagccaaacgtcatcattaagttggagcagggagaggagctgtggataacgggaggtgaatttccatgtcaacatagtcctgggatcgtgggactttaccagatcggtttgtaataacacctcgaagacgctatccgatccatcagacccagtattcctgtccgggggtacttcccacagtgtgctggaatggttatcacaagaaggctgtgctgtcccctcgcaactccaggatggtgtgtagcccagtgactgtgaggatcgcccctcctgacagaagattttcacgttctgcgataccagagcagataatcagctcaacactgtcgtcaccatcaagtaatgccccagacccatgtgcaaaggagactgtactgagtgccctcaaagagaagaagaagaaaaggacagtggaggaagaagaccaaatattccttgatggccaggaaaataaaagaaggcgccatgatagcagtggcagtggacattcagcatttgagcccctggtggccagtggagtccccgcttcttttgtgcctaagcctgggtctctgaagagaggcctcaattctcagagctcagatgaccacttgaataagagatcccgaagctcttccatgagctccttgacaggcgcttacacaagtggcatccctagctccagccgcaatgccattaccagttcctacagctccactcgaggcatctcacagctgtggaagagaaatggccccagttcatcacccttctctagcccagcctcatcccgctcccagacaccggagaggccagcaaagaaaataagagaagaagagctgtgtcatcattccagttcttcaactccattggcagcagacaaggagtcccagggagaaaaggctgcagatacaaccccaaggaagaaacaaaactcgaattctcagtctacacctggcagctctgggcagcgtaagcggaaagttcagctgctgccttctcggcgaggggaacagctgaccttgcctccacctccccagcttggctattcgatcactgccgaggacctagacttagagaagaaggcttcattacagtggttcaaccaggccttggaggacaagagtgatgctgcctcgaactctgtcactgagaccccacctaccactcagccttcatttacctttaccctgcctgctgctgcaactgcctccccacccacctccctcctggccccaagcaccaacccactgttagagagcttgaagaagatgcagactcccccgagcctgccaccctgcccagaatctgctggagcagcaaccactgaggccctctcacctccaaagacacccagcctcctacccccgctgggtttatcacagtcagggccgccagggctgctccccagcccctcctttgactccaaacccccgaccactttgctggggctgatccctgctccatccatggtaccagccactgacaccaaggcacctccaacccttcaggcagagacggctaccaaaccccaagccacatctgccccgtcccccgcccccaagcaaagcttcctgtttggaacacagaacacctcaccttccagccctgccgcccctgctgcatcttcagcatctcccatgttcaagcccattttcacggctccacccaagagtgagaaggaaggcctcacaccgcctggcccttcagtctcagccacagcgccctccagctcctccctccccacgaccaccagcaccacagccccgaccttccagcctgtctttagcagcatggggccacctgcatctgtgcccttgcctgctcccttcttcaagcagacaactactcccgccactgctcccaccacaactgccccgctcttcactggcctggccagcgccacctctgctgtggctcccatcacctctgccagtccatccacagactctgcttcgaagcctgcgtttggctttggcataaacagtgtgagcagcagcagtgtgagtaccacgaccagcaccgccactgccgcctcacagcctttcctcttcggggcgccccaggcctctgctgccagcttcaccccggccatgggctccatattccagtttggcaaacctcctgccttgcccacaaccaccacagtcaccaccttcagccagtccctgcccactgccgtgccaacggccaccagcagcagcgctgccgactttagtggttttggcagcaccctcgccacctccgccccggccaccagcagccagcccactctgacgttcagtaacacgagcacccccacgttcaacattccctttggctcaagcgccaagtccccgctcccatcatatccgggagccaacccccagcccgcatttggggccgctgaggggcagccaccgggggccgccaagccagcccttacccccagctttggcagctctttcacttttggaaactctgcagccccggccccggctactgcacccacacctgcacctgcgtccacgatcaagatcgtgcctgcgcacgtgcctacgcccatccagcctacctttggcggtgccacgcactcggcgtttggattgaaagccacggcttccgccttcggcgctcccgccagctcacagcccgcctttggcggctccactgctgtcttctccttcggtgcagccaccagctccggctttggagccaccacccagaccgccagcagcgggagcagcagctcggtgtttggcagcacaacaccatcacccttcacgtttgggggttcggcagcccccgctggcagtgggagctttgggatcaacgtggccaccccaggctccagcgccaccaccggagctttcagctttggagcaggacagagtgggagcacagccacctccacccccttcacagggggcttaggtcagaacgccctgggcaccaccggccagagcacaccgtttgccttcaacgtgggcagcacaactgagagcaaacctgtgtttggaggcaccgccacccccacctttggtcagaacacccctgcgcctggagtgggcacatcgggcagcagcctctcctttggggcatcttcagcacccgcccaaggctttgttggtgttggaccgttcggatcggcggccccttcattttccattggtgcgggatccaagaccccaggggctcgacagcgactgcaggcccgaaggcagcacacccgcaaaaagtagcctttgtcccctgtccctgttccccccaccccttccctaaatctggaccttggcacgtgctagaaagagccttggacccttccagctgcgtaaagcaaacctaccccggatctctggcttcagccgccagggggcagtggcagccctggggccctttcccttctggaggaagcacaagcctcagggaaggggaagcaggatgcggagggccaaagcccgggacctctacttgaacagttccactggggaggctggagaactaaggacctgtacatagtgtccgctgccctgactcccgcttagcgcacccttaggcaggcgccccttccacctttccccgagagccgtcgtcgctggagggggcagggtccagcccgcctggatcggtggtgtgcacctgatgggatttgggaaatgggttatccctaaagctttatcttgcttggcttagctgtgagaagtggttctcttcctctggtcccttctggggactctgtttccccatttcttgctgctgtgtccctcaccggttccttgcaggattccctcctttttaaatgcccttgaatctagctttgccttggagaccccagtgggtgctgctcctgccgttttcttcctgccaagcctgaatcaatgtttcatctccaaccctctgccagtttggcccctcagagcttggtggctcaagactgttagcctggcagagccaggggtgaagggagaagctcttggagcaggcaggatgcccactgctgcttcagctgcctcctcgcccagctaccctttggccccattgggccctcgtctgcctctccaggattgtatgtttcaagccttgtcctgtgttcctttgtctgacgctctgtgtattgctctttgaatcgagtttggaggaagagttgagttgtatgagtggcggcatgttggtagtgccggacttcctgtttcaagttttctggggcctcgctaattgaatgtggaaagtagcaccacttgacggctacaagtgccgactcctgaattttcccatggtgttctgacttcaagggctggcagccagggagaatgggcccaggggaagcaaagacctcttccctctgcggtttctgtcccacttaactgacctcactggaggctacgtcacccaaagtagatgttagaaaacctaaattaatgaaccatatttttaaaatcctatttttcccaaacagggccctctgcagcccatcctttccttccgtccttctgaaaccacataccccaggcccaagcgccttgctgtcacgcccaacctctttgggagaagtatgaatgcgtgtgtctaaattaaaagaaaaaaatatttaaacgttttttaacaaaaatttatttttgtatttaagctaaattgccttttaaattccttcaagcttggttcattgaggtggttaagtataaatgctattaactaggaattagctgtatagttaagttatgcctgtgcaaagaagaggctcaaatgctgtccccggcagctttcctgggggactagagctccttctggccatgttatatgaaatgtaattcttattttataaataatgtgatgtagatgtaaccggtgccccctccccgttgtgactgagggcgagtgttatacctggctgcgtgtgcagctgaggagggagtgaacctcaagcctaaatacctgttaggattggagggtctgggtgggcctgggcctagcaatcaagcttctacctgtaccttatgtaaggtagaccctcctagtgtcagtacctgagcttgtttacctcagttccgcaggcaggacagccggtccgggaaccctgagtgagagtgagtgtggatgtgtacagtacacacactggacggcagcgggaggctgggactttccattacaaatagagacttcattcctgttgagtctagttggaatttttagtatgaatgtgagatttttctcctgcttgtgacattaagaataaaaaactgtgatctatcgta

SEQ ID NO: 25 - DNA, 2242 bp, Homo sapiens
ggagccgggggacggcgacagcgggtcggcgggccgcaggagggggtcatgggtaaagactactaccagacgttgggcctggcccgcggcgcgtcggacgaggagatcaagcgggcctaccgccgccaggcgctgcgctaccacccggacaagaacaaggagcccggcgccgaggagaagttcaaggagatcgctgaggcctacgacgtgctcagcgacccgcgcaagcgcgagatcttcgaccgctacggggaggaaggcctaaaggggagtggccccagtggcggtagcggcggtggtgccaatggtacctctttcagctacacattccatggagaccctcatgccatgtttgctgagttcttcggtggcagaaatccctttgacaccttttttgggcagcggaacggggaggaaggcatggacattgatgacccattctctggcttccctatgggcatgggtggcttcaccaacgtgaactttggccgctcccgctctgcccaagagcccgcccgaaagaagcaagatcccccagtcacccacgaccttcgagtctcccttgaagagatctacagcggctgtaccaagaagatgaaaatctcccacaagcggctaaaccccgacggaaagagcattcgaaacgaagacaaaatattgaccatcgaagtgaagaaggggtggaaagaaggaaccaaaatcactttccccaaggaaggagaccagacctccaacaacattccagctgatatcgtctttgttttaaaggacaagccccacaatatctttaagagagatggctctgatgtcatttatcctgccaggatcagcctccgggaggctctgtgtggctgcacagtgaacgtccccactctggacggcaggacgatacccgtcgtattcaaagatgttatcaggcctggcatgcggcgaaaagttcctggagaaggcctccccctccccaaaacacccgagaaacgtggggacctcattattgagtttgaagtgatcttccccgaaaggattccccagacatcaagaaccgtacttgagcaggttcttccaatatagctatctgagctccccaaggactgaccagggacctttccagagctcaaggatttctggacctttctaccagttgtggaccatgagagggtgggagggcccagggagggctttcgtactgctgaatgttttccagagcatatattacaatctttcaaagtcgcacactagacttcagtggtttttcgagctatagggcatcaggtggtgggaacagcaggaaaaggcattccagtctgccccactgggtctggcagccctcccgggatgggcccacatccacctccagtccctggccaggggtgagaggcagaccagcagatggacttgatccctctgtgtctttttgcttctggctggtagataatgtcaacctgcagtcttgattcccagaccctgtacactcctccttttctgttgtgtgatcagtttgtgctttattctgtatttgtctcccatgtcttgctcttctcctggagaattctgtcttctctttggccatctcaaattgagaacctaaactattcctgcagaactgcctggttggcgtccacaagcaatacctctcgttccagcaggaccaagggagccagcctccagtgagtgactccagcaagtgcagccacctctcccttgatggtctgggagcctggcctcagcaaggggccttcctgacctctggctccagtgaagctgaatgtcctcactttgtgggtcacactctttacatttctgtaaggcaatcttggcacacgtggggcttaccagtggcccaggtaattttttgtttcatggactatggactctttcaaagggatctgatccttttgaattttgcacagccctagatacaatcccttttgataaaagggtctttgcttctgattacaggagcactgtggaacgtctgtaaatatgtttttataattccatgtatagttggtgtacactcaaaacctgtccccggcagccagtgctctctgtatagggccataatggaattctgaagaaatcttggggagggaaggggagttggaacaaatgtctgttccctggaggccagtccagtgctcagacctttagactcattgtaagttgccactgccaacatgagaccaaagtgtgtgactagtcaatgaagtgcgacagcattaaagactgatgctaaacctca

SEQ ID NO: 26 - DNA, 2270 bp, Homo sapiens
gtgcatgggggtgggggagccgggggtgacgacggggacggcgcggcggagcccgctgcggacccgggctcacctgggctcggccgccggggtccgcggggcggcgcctccggctcagctgcggggcgaggggttgtgaatgcaggagccgaccccgttcgtgggcttgggggctgggttgggataattcccgggaagtgatgacctggccccggcagggcacgcaggaggcagcggccgcccaggtccggagagcggggccgcctcggagggcctaaaggggagtggccccagtggcggtagcggcggtggtgccaatggtacctctttcagctacacattccatggagaccctcatgccatgtttgctgagttcttcggtggcagaaatccctttgacaccttttttgggcagcggaacggggaggaaggcatggacattgatgacccattctctggcttccctatgggcatgggtggcttcaccaacgtgaactttggccgctcccgctctgcccaagagcccgcccgaaagaagcaagatcccccagtcacccacgaccttcgagtctcccttgaagagatctacagcggctgtaccaagaagatgaaaatctcccacaagcggctaaaccccgacggaaagagcattcgaaacgaagacaaaatattgaccatcgaagtgaagaaggggtggaaagaaggaaccaaaatcactttccccaaggaaggagaccagacctccaacaacattccagctgatatcgtctttgttttaaaggacaagccccacaatatctttaagagagatggctctgatgtcatttatcctgccaggatcagcctccgggaggctctgtgtggctgcacagtgaacgtccccactctggacggcaggacgatacccgtcgtattcaaagatgttatcaggcctggcatgcggcgaaaagttcctggagaaggcctccccctccccaaaacacccgagaaacgtggggacctcattattgagtttgaagtgatcttccccgaaaggattccccagacatcaagaaccgtacttgagcaggttcttccaatatagctatctgagctccccaaggactgaccagggacctttccagagctcaaggatttctggacctttctaccagttgtggaccatgagagggtgggagggcccagggagggctttcgtactgctgaatgttttccagagcatatattacaatctttcaaagtcgcacactagacttcagtggtttttcgagctatagggcatcaggtggtgggaacagcaggaaaaggcattccagtctgccccactgggtctggcagccctcccgggatgggcccacatccacctccagtccctggccaggggtgagaggcagaccagcagatggacttgatccctctgtgtctttttgcttctggctggtagataatgtcaacctgcagtcttgattcccagaccctgtacactcctccttttctgttgtgtgatcagtttgtgctttattctgtatttgtctcccatgtcttgctcttctcctggagaattctgtcttctctttggccatctcaaattgagaacctaaactattcctgcagaactgcctggttggcgtccacaagcaatacctctcgttccagcaggaccaagggagccagcctccagtgagtgactccagcaagtgcagccacctctcccttgatggtctgggagcctggcctcagcaaggggccttcctgacctctggctccagtgaagctgaatgtcctcactttgtgggtcacactctttacatttctgtaaggcaatcttggcacacgtggggcttaccagtggcccaggtaattttttgtttcatggactatggactctttcaaagggatctgatccttttgaattttgcacagccctagatacaatcccttttgataaaagggtctttgcttctgattacaggagcactgtggaacgtctgtaaatatgtttttataattccatgtatagttggtgtacactcaaaacctgtccccggcagccagtgctctctgtatagggccataatggaattctgaagaaatcttggggagggaaggggagttggaacaaatgtctgttccctggaggccagtccagtgctcagacctttagactcattgtaagttgccactgccaacatgagaccaaagtgtgtgactagtcaatgaagtgcgacagcattaaagactgatgctaaacctcaggggaaaaaaaaaaa

SEQ ID NO: 27 - DNA, 7470 bp, Homo sapiens
agatattaatcacggagttccagggagaaggaacttgtgaaatgggggagccggctggggttgccggcaccatggagtcaccttttagcccgggactctttcacaggctggatgaagattgggattctgctctctttgctgaactcggttatttcacagacactgatgagctgcaattggaagcagcaaatgagacgtatgaaaacaattttgataatcttgattttgatttggatttgatgccttgggagtcagacatttgggacatcaacaaccaaatctgtacagttaaagatattaaggcagaacctcagccactttctccagcctcctcaagttattcagtctcgtctcctcggtcagtggactcttattcttcaactcagcatgttcctgaggagttggatttgtcttctagttctcagatgtctcccctttccttatatggtgaaaactctaatagtctctcttcagcggagccactgaaggaagataagcctgtcactggtcctaggaacaagactgaaaatggactgactccaaagaaaaaaattcaggtgaattcaaaaccttcaattcagcccaagcctttattgcttccagcagcacccaagactcaaacaaactccagtgttccagcaaaaaccatcattattcagacagtaccaacgcttatgccattggcaaagcagcaaccaattatcagtttacaacctgcacccactaaaggccagacggttttgctgtctcagcctactgtggtacaacttcaagcacctggagttctgccctctgctcagccagtccttgctgttgctgggggagtcacacagctccctaatcacgtggtgaatgtggtaccagccccttcagcgaatagcccagtgaatggaaaactttccgtgactaaacctgtcctacaaagtaccatgagaaatgtcggttcagatattgctgtgctaaggagacagcaacgtatgataaaaaatcgagaatccgcttgtcagtctcgcaagaagaagaaagaatatatgctagggttagaggcgagattaaaggctgccctctcagaaaacgagcaactgaagaaagaaaatggaacactgaagcggcagctggatgaagttgtgtcagagaaccagaggcttaaagtccctagtccaaagcgaagagttgtctgtgtgatgatagtattggcatttataatactgaactatggacctatgagcatgttggaacaggattccaggagaatgaaccctagtgtgagccctgcaaatcaaaggaggcaccttctaggattttctgctaaagaggcacaggacacatcagatggtattatccagaaaaacagctacagatatgatcattctgtttcaaatgacaaagccctgatggtgctaactgaagaaccattgctttacattcctccacctccttgtcagcccctaattaacacaacagagtctctcaggttaaatcatgaacttcgaggatgggttcatagacatgaagtagaaaggaccaagtcaagaagaatgacaaataatcaacagaaaacccgtattcttcagggtgctctggaacagggctcaaattctcagctgatggctgttcaatacacagaaaccactagtagtatcagcaggaactcagggagtgagctacaagtgtattatgcttcacccagaagttatcaagacttttttgaagccatccgcagaaggggagacacattttatgttgtgtcatttcgaagggatcacctgctgttaccagctaccacccataacaagaccacaagaccaaaaatgtcaattgtgttaccagcaataaacataaatgagaatgtgatcaatgggcaggactacgaagtgatgatgcagattgactgtcaggtgatggacaccaggatcctccatatcaaaagttcgtcagttcctccttacctccgagatcagcagaggaatcaaaccaacaccttctttggctcccctcccgcagccacagaggcaacccacgttgtcagcaccatccctgagtcattacaatagcaccctgcagctatgctggaaaactgagcgtgggaccctgccagactgaagagcaggtgagcaaaatgctgctttctgccttggtggcaggcagagaactgtctcgtactagaattcaaggaggaaagaagaagaaataaaagaagctgctccatttttcatcatctacccatctatttggaaagcactggaattcagatgcaagagaacaatgtttcttcagtggcaaatgtagccctgcatcctccagtgttacctggtgtagatttttttttctgtacctttctaaacctctcttccctctgtgatggttttgtgtttaaacagtcatcttcttttaaataatatccacctctcctttttgccatttcacttattgattcataaagtgaattttatttaaagctatgccacacatgcatgttcaaatggtttccactgattcgatttttcattcatttaatgcaaacccattctggatattgtgcttatttgagaaaacacatttcaaaaccagaaaagccaaaaacactccaaaaacaagcaaaacaatttggagctttagataaaaggaaaaactcccagttggtaaagtttatctttacttaggatttgtggctcacacctaaacaaagggggtcagggagtgggtacaaatttgagaaaatagaagggtaagggaagggccagtggtggggtttggagagaggagatagctccattaatacacatgtttaaaagatggaaagttcacgcctgtaatcccagcactttgggaggccgaggcgggtggatcacgaggtcaggagatcaagaccatcccggctaaaacggtgaaaccccgtctctactaaaaatacaaaaaattagccgggcgtagtgacgggcgcctgtagtcccagctacttgggaggctgaggcaggagaatggcgtgaacccgggaggcggagcttgcagtgagccgagatcccgccactgcactccagcctgggcgacagagcgagactccgtctcaaaaaaaaaaaaaaaaaaaaaaaaagatggaaagttcgatgtgactgcagtatgagattaaagccacaactattgtttattttggggactctaggccaccaagtattagcacacatacttatgttttctctactaatctggtccaggtcctcatggaccacaggacaaagctttcattttcattcattcttctattgaaattataccaaattcagctgaggaatatggaagtaactttagacttaaacaagacaaaagttttttcactgaagaattgacaagtatttgctccttaaaacaacgcagattagtgaacgtggattcctgctgagggagtgcatcccataatatggcaataattttcagtttctccaacgaaaagatagtgaaggaattaaatcttttgtcctcccatggttaaaaaaaaaaaaaaagctgtgttcatttttactgtactatgcctcttttttcaccatagtagacaattatgtttcatttgatgaattcatagaactggatctcatacagcgatgtcctctctaatgttctacctttcagtttctaaagtgagtcttcctccctctcctacaaaacttttcaattttttgatgtaactcatctacaaatactgtttcttaccccagttgacttgcctttgtcagatttcttcttgttccacactatagcaatcaatttctcttcttccttacaagaaagggaacgagaaattgtagcaacctctcaaggattatatgcagctagttagttttctgcctgtgaaattaggtctggctcctaaataattttaaagaaccatcagcacttctaactctctggacaggtgcctctttgtccaagctagttaaatgctttccaaggaaatcagttcaacttttgtgagcggggaaaagcagggctttattgttgtgttacctgggagtctggagtttgaaaagtgctaattaaccttcctctttttccacattacaaacctttttaagcagcgcagcactccccttagatttggctatcctgggtgattttcagacaagaaccattttctctggggaccattcttctgctgggtgccaaggaatataaggcaaatgcccagaagaccttcaggtgactgggcagtcttatcatgggatatttcttctggccctgccccttcccattctgtaatgtgaattagccacaccagaggctgtgaccatggctagtagacagtggcaacatagtcatccccaagatgctaatcttctgctggaactgtcatacgttatcatggtcaatgtaaacctggtttgtgtggggtgattataaatagagtttccctcctctctgtgacagaatcacaggagaaggacccatctcgtggccttcttgttcttagcgcttcacttttacttcatccctcgattcccagctttttctatcatcattttgccaactcctcagatgcaagactttggttatgtcatactcaccaacgttagtccctctcttccaggtgaaaaggtgggtagcggttgggagggagtctccactgaagagcaggaaggtggtagcagggccggcagctctgccacagagctaggggtgcctgtaaggtgccgcctagagcagcctgggagctttgccttcttttgtctctcactagcccttctactctttgtcattgcctgttcttgagtggatctttgaaatgaggggacaggattctcctaagggtagagtttcaggaaatgagtgaaaggcaattgacaaatgcaaagaagtagtcactttttaaattgctggcaaagctataattaatccctaggcacaattgtagtttttattttaatgtttgtatgcacaaggccctttaggaaatgagaagttgccatgccagattaatttttttttttttttttggtgggattgccttttgggggttgcagccagaaattgtgggtaatgtgtgtatttttttatttattaaattttaaacaggattgtgcaagcttatgagacaattagataaactcatggaggaggcaggtcctcctgttattagatgattttgtgctcttggggctgacaataatacactcttgggaagtgatggtagagactgatgggaatagtctttctgcctggttgcaagtcccaaatttttaagggttaatggaagtaagtggatgtttcctcatgttaactactgaatcagatgttaggagcttgtccctttggggttgacttatgcccagcagtacagggacacagcttcattagagtgttagtgtaaactaactccaaagttaggagttaatgtgaaaggatcatccttgaaacaaatctgctgtttgccatgcttgtagtacagaaacttcacatggagttttgggtgggatttgtgttttcacaagtaaaaaatccctcacgattataaaactcagagcatcatctaatttttttttttaatgactacaagttccagcacaaaactggcatttctttgccatttcttgccagtaagaagttgacacggaggtatttgaaagcaatgttatgtgagtcattcttaagtgttccaagtaagtttagaaacagaaaaggaacttgggattcaaattgatttttcaaatcatttttaaagagacatcatcctgactaaatcttagcctgaaccttcctcccctgtgtgtattccccggtagtcaccgcagcgagatgctggtgagactgccgtggtggcatttagcatcgttaaaactggaaaactctcaagctctttgccactttcctactattttttgattcttgccattttaccaagcttaggttgtgaaacttgacagaaatgtattacaggaaaaacttataattgtatttgactttctaacacattgcaaagtttcaaagtgactttcactttcaacaacatattagaagtaaccacttttgctttcacagcctgaagagttagagcctgatctgatgccccctttcactctgaagtcatgggaaattttccagccatgaaagccctctttccactgcatactgatgggctgactcagcttccttcagccgactgagatcttttcatactattggctatttcataccaattaacctcttaaataagattgtgaattgccaaaattgatagacacttattaccacctgtggactccatattccttaccacaaatgttattttcatcagtcctgagtcattttaacttacagaaattaggattgttgctgctaatatgaataccaattataacttttagaaacaagaataaagcctaaaagagaatgaaatataagaaatgttcgttcccacccctaataacatttggaagtgaatattcccattttcttccacccacagggattgggattgatttttaatttcctaggaaacaatactagactacccaaaaagatgttgccagaatccaaaaggaactatgctcgtaaaagaaatgcagttttctcctacctaaaaaaaagaaagtaaagtgtgttctgttcttatctttttaatgactaagctttaaacagtttattttgggtaagactagaactttcggccatttgttctaatatgtgtgttattagatgcaatagaatttatgaaaagaagaatgacaaaggtatctgattagaaaatttgatcttacgcatgaatccatgtcatggccagccactgtcacatagtgggtgccattctcaacatattggtttgctaactttaagcattagggatttagcacactaaaatacttttaattatattaggtttggtaactaaggagtaaataaatcataatttatcatttgccaaggccaacaaacaacactattgtgctgtttgctctcaatgaagttgaataaaccaggaggcttggcatatcccctttatgttaatcccagctagagattagtaggttgactttcacagcaattgtatattgatccattttaactcatccttgccataatttccaggccagtcaccaggacagaggagatgatggggaaacagagctttagatgaaaactactatgcactactagccttagaggcactggtttcctgttaccactttggcaagtatggatggtctaagtccagtagggcttcatccatggagccattagaactgaggggggagtgttagagatgccatttcaccaggatctttttgctcaggttgtacccatgccaattgaagaacgtgttaaagatgaggaggagagatgtaccattctctcccttaataatgatgttggtttgcaaaacctaaagaaataataacaacagactatttcatactttcaagcaagtctttatactacctgttatttctctaaaattcaaataaagaatttttaaactta

SEQ ID NO: 28 - DNA, 2041 bp, Homo sapiens
agccatttctactttgcccgcccacagatgtagttttctctgcgcgtgtgcgttttccctcctccccgccctcagggtccacggccaccatggcgtattaggggcagcagtgcctgcggcagcattggcctttgcagcggcggcagcagcaccaggctctgcagcggcaacccccagcggcttaagccatggcgtgagtaccggggcgggtcgtccagctgtgctcctggggccggcgcgggttttggattggtggggtgcggcctggggccagggcggtgccgccaagggggaagcgatttaacgagcgcccgggacgcgtggtctttgcttgggtgtccccgagacgctcgcgtgcctgggatcgggaaagcgtagtcgggtgcccggactgcttccccaggagccctacagccctcggaccccgagccccgcaagggtcccaggggtcttggctgttgccccacgaaacgtggcaggaaccaagatggcggcggcagggcggcggcgcgggcgtgagtcaagggcgggcggtgggcggggcgcggccgccctggccgtatttggacgtggggacggagcgctttcctcttggcggccggtggaagaatcccctggtctccgtgagcgtccattttgtggaacctgagttgcaagcagggaggggcaaatacaactgccctgttcccgattctctagatggccgatctagagaagtcccgcctcataagtggaaggatgaaattctcagaacagctaacctctaatgggagttggcttctgattctcattcaggcttctcacggcattcagcagcagcgttgctgtaaccgacaaagacaccttcgaattaagcacattcctcgattccagcaaagcaccgcaacatgaccgaaatgagcttcctgagcagcgaggtgttggtgggggacttgatgtcccccttcgaccagtcgggtttgggggctgaagaaagcctaggtctcttagatgattacctggaggtggccaagcacttcaaacctcatgggttctccagcgacaaggctaaggcgggctcctccgaatggctggctgtggatgggttggtcagtccctccaacaacagcaaggaggatgccttctccgggacagattggatgttggagaaaatggatttgaaggagttcgacttggatgccctgttgggtatagatgacctggaaaccatgccagatgaccttctgaccacgttggatgacacttgtgatctctttgcccccctagtccaggagactaataagcagcccccccagacggtgaacccaattggccatctcccagaaagtttaacaaaacccgaccaggttgcccccttcaccttcttacaacctcttcccctttccccaggggtcctgtcctccactccagatcattcctttagtttagagctgggcagtgaagtggatatcactgaaggagataggaagccagactacactgcttacgttgccatgatccctcagtgcataaaggaggaagacaccccttcagataatgatagtggcatctgtatgagcccagagtcctatctggggtctcctcagcacagcccctctaccaggggctctccaaataggagcctcccatctccaggtgttctctgtgggtctgcccgtcccaaaccttacgatcctcctggagagaagatggtagcagcaaaagtaaagggtgagaaactggataagaagctgaaaaaaatggagcaaaacaagacagcagccactaggtaccgccagaagaagagggcggagcaggaggctcttactggtgagtgcaaagagctggaaaagaagaacgaggctctaaaagagagggcggattccctggccaaggagatccagtacctgaaagatttgatagaagaggtccgcaaggcaagggggaagaaaagggtcccctagttgaggatagtcaggagcgtcaatgtgcttgtacatagagtgctgtagctgtgtgttccaataaattattttgtagggaaagtaaaaaaaaaaaaaaa

SEQ ID NO: 29 - DNA, 4536 bp, Homo sapiens
aggctgtcactcaggtggcagcggcagaggccgggctgagacgtggccaggggaacacggctggctgtccaggccgtcggggcggcagtagggtccctagcacgtccttgccttcttgggagctccaagcggcgggagaggcaggcgtcagtggctgcgcctccatgcctgcgcgcggggcgggacgctgatggagcgcgccatcagcccggggctgctggtacgggcgctgctgctgctgctgctgctgctggggctcgcggcaaggacggtggccgcggggcgcgcccgtggcctcccagcgccgacggcggaggcggcgttcggcctcggggcggccgctgctcccacctcagcgacgcgagtaccggcggcgggcgccgtggctgcggccgaggtgactgtggaggacgctgaggcgctgccggcagccgcgggagagcaggagcctcggggtccggaaccagacgatgagacagagttgcgaccgcgcggcaggtcattagtaattatcagcactttagatgggagaattgctgccttggatcctgaaaatcatggtaaaaagcagtgggatttggatgtgggatccggttccttggtgtcatccagccttagcaaaccagaggtatttgggaataagatgatcattccttccctggatggagccctcttccagtgggaccaagaccgtgaaagcatggaaacagttcctttcacagttgaatcacttcttgaatcttcttataaatttggagatgatgttgttttggttggaggaaaatctctgactacatatggactcagtgcatatagtggaaaggtgaggtatatctgttcagctctgggttgtcgccaatgggatagtgacgaaatggaacaagaggaagacatcctgcttctacagcgtacccaaaaaactgttagagctgtcggacctcgcagtggcaatgagaagtggaatttcagtgttggccactttgaacttcggtatattccagacatggaaacgagagccggatttattgaaagcacctttaagcccaatgagaacacagaagagtctaaaattatttcagatgtggaagaacaggaagctgccataatggacatagtgataaaggtttcggttgctgactggaaagttatggcattcagtaagaagggaggacatctggaatgggagtaccagttttgtactccaattgcatctgcctggttacttaaggatgggaaagtcattcccatcagtctttttgatgatacaagttatacatctaatgatgatgttttagaagatgaagaagacattgtagaagctgccagaggagccacagaaaacagtgtttacttgggaatgtatagaggccagctgtatctgcagtcatcagtcagaatttcagaaaagtttccttcaagtcccaaggctttggaatctgtcactaatgaaaacgcaattattcctttaccaacaatcaaatggaaacccttaattcattctccttccagaactcctgtcttggtaggatctgatgaatttgacaaatgtctcagtaatgataagttttctcatgaagaatatagtaatggtgcactttcaatcttgcagtatccatatgataatggttattatctaccatactacaagagggagaggaacaaacgaagcacacagattacagtcagattcctcgacaacccacattacaacaagaatatccgcaaaaaggatcctgttcttcttttacactggtggaaagaaatagttgcaacgattttgttttgtatcatagcaacaacgtttattgtgcgcaggcttttccatcctcatcctcacaggcaaaggaaggagtctgaaactcagtgtcaaactgaaaataaatatgattctgtaagtggtgaagccaatgacagtagctggaatgacataaaaaactctggatatatatcacgatatctaactgattttgagccaattcaatgcctgggacgtggtggctttggagttgtttttgaagctaaaaacaaagtagatgactgcaattatgctatcaagaggatccgtctccccaatagggaattggctcgggaaaaggtaatgcgagaagttaaagccttagccaagcttgaacacccgggcattgttagatatttcaatgcctggctcgaagcaccaccagagaagtggcaagaaaagatggatgaaatttggctgaaagatgaaagcacagactggccactcagctctcctagcccaatggatgcaccatcagttaaaatacgcagaatggatcctttcgctacaaaagaacatattgaaatcatagctccttcaccacaaagaagcaggtctttttcagtagggatttcctgtgaccagacaagttcatctgagagccagttctcaccactggaattctcaggaatggaccatgaggacatcagtgagtcagtggatgcagcatacaacctccaggacagttgccttacagactgtgatgtggaagatgggactatggatggcaatgatgaggggcactcctttgaactttgtccttctgaagcttctccttatgtaaggtcaagggagagaacctcctcttcaatagtatttgaagattctggctgtgataatgcttccagtaaagaagagccgaaaactaatcgattgcatattggcaaccattgtgctaataaactaactgctttcaagcccaccagtagcaaatcttcttctgaagctacattgtctatttctcctccaagaccaaccactttaagtttagatctcactaaaaacaccacagaaaaactccagcccagttcaccaaaggtgtatctttacattcaaatgcagctgtgcagaaaagaaaacctcaaagactggatgaatggacgatgtaccatagaggagagagagaggagcgtgtgtctgcacatcttcctgcagatcgcagaggcagtggagtttcttcacagtaaaggactgatgcacagggacctcaagccatccaacatattctttacaatggatgatgtggtcaaggttggagactttgggttagtgactgcaatggaccaggatgaggaagagcagacggttctgaccccaatgccagcttatgccagacacacaggacaagtagggaccaaactgtatatgagcccagagcagattcatggaaacagctattctcataaagtggacatcttttctttaggcctgattctatttgaattgctgtatccattcagcactcagatggagagagtcaggaccttaactgatgtaagaaatctcaaatttccaccattatttactcagaaatatccttgtgagtacgtgatggttcaagacatgctctctccatcccccatggaacgacctgaagctataaacatcattgaaaatgctgtatttgaggacttggactttccaggaaaaacagtgctcagacagaggtctcgctccttgagttcatcgggaacaaaacattcaagacagtccaacaactcccatagccctttgccaagcaattagccttaagttgtgctagcaaccctaataggtgatgcagataatagcctacttcttagaatatgcctgtccaaaattgcagacttgaaaagtttgttcttcgctcaatttttttgtggactactttttttatatcaaatttaagctggatttgggggcataacctaatttgagccaactcctgagttttgctatacttaaggaaagggctatctttgttctttgttagtctcttgaaactggctgctggccaagctttatagccctcaccatttgcctaaggaggtagcagcaatccctaatatatatatatagtgagaactaaaatggatatatttttataatgcagaagaaggaaagtccccctgtgtggtaactgtattgttctagaaatatgctttctagagatatgatgattttgaaactgatttctagaaaaagctgactccatttttgtccctggcgggtaaattaggaatctgcactattttggaggacaagtagcacaaactgtataacggtttatgtccgtagttttatagtcctatttgtagcattcaatagctttattccttagatggttctagggtgggtttacagctttttgtacttttacctccaataaagggaaaatgaagctttttatgtaaattggttgaaaggtctagttttgggaggaaaaaagccgtagtaagaaatggatcatatatattacaactaacttcttcaactatggactttttaagcctaatgaaatcttaagtgtcttatatgtaatcctgtaggttggtacttcccccaaactgattataggtaacagtttaatcatctcacttgctaacatgtttttatttttcactgtaaatatgtttatgttttatttataaaaattctgaaatcaatccatttgggttggtggtgtacagaacacacttaagtgtgttaacttgtgacttctttcaagtctaaatgatttaataaaactttttttaaattaa

SEQ ID NO: 30 - DNA, 4233 bp, Homo sapiens
agttcagatgagaggtttagtgtaaagtggtataggacagaggggacagagttgagatatttgatgcaaaatctaggttcctggcaagtgaatgcattcttcctttcttggtacccttgtccagagattgagactgcgtggccagaagttgacaaaagaggtttaagggagcagggaagaaaaggtcattagtaattatcagcactttagatgggagaattgctgccttggatcctgaaaatcatggtaaaaagcagtgggatttggatgtgggatccggttccttggtgtcatccagccttagcaaaccagaggtatttgggaataagatgatcattccttccctggatggagccctcttccagtgggaccaagaccgtgaaagcatggaaacagttcctttcacagttgaatcacttcttgaatcttcttataaatttggagatgatgttgttttggttggaggaaaatctctgactacatatggactcagtgcatatagtggaaaggtgaggtatatctgttcagctctgggttgtcgccaatgggatagtgacgaaatggaacaagaggaagacatcctgcttctacagcgtacccaaaaaactgttagagctgtcggacctcgcagtggcaatgagaagtggaatttcagtgttggccactttgaacttcggtatattccagacatggaaacgagagccggatttattgaaagcacctttaagcccaatgagaacacagaagagtctaaaattatttcagatgtggaagaacaggaagctgccataatggacatagtgataaaggtttcggttgctgactggaaagttatggcattcagtaagaagggaggacatctggaatgggagtaccagttttgtactccaattgcatctgcctggttacttaaggatgggaaagtcattcccatcagtctttttgatgatacaagttatacatctaatgatgatgttttagaagatgaagaagacattgtagaagctgccagaggagccacagaaaacagtgtttacttgggaatgtatagaggccagctgtatctgcagtcatcagtcagaatttcagaaaagtttccttcaagtcccaaggctttggaatctgtcactaatgaaaacgcaattattcctttaccaacaatcaaatggaaacccttaattcattctccttccagaactcctgtcttggtaggatctgatgaatttgacaaatgtctcagtaatgataagttttctcatgaagaatatagtaatggtgcactttcaatcttgcagtatccatatgataatggttattatctaccatactacaagagggagaggaacaaacgaagcacacagattacagtcagattcctcgacaacccacattacaacaagaatatccgcaaaaaggatcctgttcttcttttacactggtggaaagaaatagttgcaacgattttgttttgtatcatagcaacaacgtttattgtgcgcaggcttttccatcctcatcctcacaggcaaaggaaggagtctgaaactcagtgtcaaactgaaaataaatatgattctgtaagtggtgaagccaatgacagtagctggaatgacataaaaaactctggatatatatcacgatatctaactgattttgagccaattcaatgcctgggacgtggtggctttggagttgtttttgaagctaaaaacaaagtagatgactgcaattatgctatcaagaggatccgtctccccaatagggaattggctcgggaaaaggtaatgcgagaagttaaagccttagccaagcttgaacacccgggcattgttagatatttcaatgcctggctcgaagcaccaccagagaagtggcaagaaaagatggatgaaatttggctgaaagatgaaagcacagactggccactcagctctcctagcccaatggatgcaccatcagttaaaatacgcagaatggatcctttcgctacaaaagaacatattgaaatcatagctccttcaccacaaagaagcaggtctttttcagtagggatttcctgtgaccagacaagttcatctgagagccagttctcaccactggaattctcaggaatggaccatgaggacatcagtgagtcagtggatgcagcatacaacctccaggacagttgccttacagactgtgatgtggaagatgggactatggatggcaatgatgaggggcactcctttgaactttgtccttctgaagcttctccttatgtaaggtcaagggagagaacctcctcttcaatagtatttgaagattctggctgtgataatgcttccagtaaagaagagccgaaaactaatcgattgcatattggcaaccattgtgctaataaactaactgctttcaagcccaccagtagcaaatcttcttctgaagctacattgtctatttctcctccaagaccaaccactttaagtttagatctcactaaaaacaccacagaaaaactccagcccagttcaccaaaggtgtatctttacattcaaatgcagctgtgcagaaaagaaaacctcaaagactggatgaatggacgatgtaccatagaggagagagagaggagcgtgtgtctgcacatcttcctgcagatcgcagaggcagtggagtttcttcacagtaaaggactgatgcacagggacctcaagccatccaacatattctttacaatggatgatgtggtcaaggttggagactttgggttagtgactgcaatggaccaggatgaggaagagcagacggttctgaccccaatgccagcttatgccagacacacaggacaagtagggaccaaactgtatatgagcccagagcagattcatggaaacagctattctcataaagtggacatcttttctttaggcctgattctatttgaattgctgtatccattcagcactcagatggagagagtcaggaccttaactgatgtaagaaatctcaaatttccaccattatttactcagaaatatccttgtgagtacgtgatggttcaagacatgctctctccatcccccatggaacgacctgaagctataaacatcattgaaaatgctgtatttgaggacttggactttccaggaaaaacagtgctcagacagaggtctcgctccttgagttcatcgggaacaaaacattcaagacagtccaacaactcccatagccctttgccaagcaattagccttaagttgtgctagcaaccctaataggtgatgcagataatagcctacttcttagaatatgcctgtccaaaattgcagacttgaaaagtttgttcttcgctcaatttttttgtggactactttttttatatcaaatttaagctggatttgggggcataacctaatttgagccaactcctgagttttgctatacttaaggaaagggctatctttgttctttgttagtctcttgaaactggctgctggccaagctttatagccctcaccatttgcctaaggaggtagcagcaatccctaatatatatatatagtgagaactaaaatggatatatttttataatgcagaagaaggaaagtccccctgtgtggtaactgtattgttctagaaatatgctttctagagatatgatgattttgaaactgatttctagaaaaagctgactccatttttgtccctggcgggtaaattaggaatctgcactattttggaggacaagtagcacaaactgtataacggtttatgtccgtagttttatagtcctatttgtagcattcaatagctttattccttagatggttctagggtgggtttacagctttttgtacttttacctccaataaagggaaaatgaagctttttatgtaaattggttgaaaggtctagttttgggaggaaaaaagccgtagtaagaaatggatcatatatattacaactaacttcttcaactatggactttttaagcctaatgaaatcttaagtgtcttatatgtaatcctgtaggttggtacttcccccaaactgattataggtaacagtttaatcatctcacttgctaacatgtttttatttttcactgtaaatatgtttatgttttatttataaaaattctgaaatcaatccatttgggttggtggtgtacagaacacacttaagtgtgttaacttgtgacttctttcaagtctaaatgatttaataaaactttttttaaattaagaaaaaaaaaa

SEQ ID NO: 31 - DNA, 4737 bp, Homo sapiens
ggtttgaaaggaaggcagagagggcactgggaggaggcagtgggagggcggagggcgggggccttcggggtgggcgcccagggtagggcaggtggccgcggcgtggaggcagggagaatgcgactctccaaaaccctcgtcgacatggacatggccgactacagtgctgcactggacccagcctacaccaccctggaatttgagaatgtgcaggtgttgacgatgggcaatgacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggagggtcccccagcgatgcaccccatgcccaccaccccctgcaccctcacctgatgcaggaacatatgggaaccaacgtcatcgttgccaacacaatgcccactcacctcagcaacggacagatgtgtgagtggccccgacccaggggacaggcagccacccctgagaccccacagccctcaccgccaggtggctcagggtctgagccctataagctcctgccgggagccgtcgccacaatcgtcaagcccctctctgccatcccccagccgaccatcaccaagcaggaagttatctagcaagccgctggggcttgggggctccactggctccccccagccccctaagagagcacctggtgatcacgtggtcacggcaaaggaagacgtgatgccaggaccagtcccagagcaggaatgggaaggatgaagggcccgagaacatggcctaagggccacatcccactgccacccttgacgccctgctctggataacaagactttgacttggggagacctctactgccttggacaacttttctcatgttgaagccactgccttcaccttcaccttcatccatgtccaacccccgacttcatcccaaaggacagccgcctggagatgacttgaggccttacttaaacccagctcccttcttccctagcctggtgcttctcctctcctagcccctgtcatggtgtccagacagagccctgtgaggctgggtccaattgtggcacttggggcaccttgctcctccttctgctgctgcccccacctctgctgcctccctctgctgtcaccttgctcagccatcccgtcttctccaacaccacctctccagaggccaaggaggccttggaaacgattcccccagtcattctgggaacatgttgtaagcactgactgggaccaggcaccaggcagggtctagaaggctgtggtgagggaagacgcctttctcctccaacccaacctcatcctccttcttcagggacttgggtgggtacttgggtgaggatccctgaaggccttcaacccgagaaaacaaacccaggttggcgactgcaacaggaacttggagtggagaggaaaagcatcagaaagaggcagaccatccaccaggcctttgagaaagggtagaattctggctggtagagcaggtgagatgggacattccaaagaacagcctgagccaaggcctagtggtagtaagaatctagcaagaattgaggaagaatggtgtgggagagggatgatgaagagagagagggcctgctggagagcatagggtctggaacaccaggctgaggtcctgatcagcttcaaggagtatgcagggagctgggcttccagaaaatgaacacagcagttctgcagaggacgggaggctggaagctgggaggtcaggtggggtggatgatataatgcgggtgagagtaatgaggcttggggctggagaggacaagatgggtaaaccctcacatcagagtgacatccaggaggaataagctcccagggcctgtctcaagctcttccttactcccaggcactgtcttaaggcatctgacatgcatcatctcatttaatcctcccttcctccctattaacctagagattgtttttgttttttattctcctcctccctccccgccctcacccgccccactccctcctaacctagagattgttacagaagctgaaattgcgttctaagaggtgaagtgattttttttctgaaactcacacaactaggaagtggctgagtcaggacttgaacccaggtctccctggatcagaacaggagctcttaactacagtggctgaatagcttctccaaaggctccctgtgttctcaccgtgatcaagttgaggggcttccggctcccttctacagcctcagaaaccagactcgttcttctgggaaccctgcccactcccaggaccaagattggcctgaggctgcactaaaattcacttagggtcgagcatcctgtttgctgataaatattaaggagaattcatgactcttgacagcttttctctcttcactccccaagtcaaggggaggggtggcaggggtctgtttcctggaagtcaggctcatctggcctgttggcatgggggtgggacagtgtgcacagtgtgggggcaggggagggctaagcaggcctgggtttgagggctgctccggagaccgtcactccaggtgcattctggaagcattagaccccaggatggagcgaccagcatgtcatccatgtggaatcttggtggctttgaggacattctggaaaatgccactgaccagtgtgaacaaaagggatgtgttatggggctggaggtgtgattaggtaggagggaaactgttggaccgactcctgccccctgctcaacactgacccctctgagtggttggaggcagtgccccagtgcccagaaatcccaccattagtgattgttttttatgagaaagaggcgtggagaagtattggggcaatgtgtcagggaggaatcaccacatccctacggcagtcccagccaagcccccaatcccagcggagactgtgccctgctcagagctcccaagccttcccccaccacctcactcaagtgcccctgaaatccctgccagacggctcagcctggtctgcggtaaggcagggaggctggaaccatttctgggcattgtggtcattcccactgtgttcctccacctcctccctccagcgttgctcagacctctgtcttgggagaaaggttgagataagaatgtcccatggagtgccgtgggcaacagtggcccttcatgggaacaatctgttggagcagggggtcagttctctgctgggaatctacccctttctggaggagaaacccattccaccttaataactttattgtaatgtgagaaacacaaaacaaagtttacttttttgactctaagctgacatgatattagaaaatctctcgctctcttttttttttttttttttttttttggctacttgagttgtggtcctaaaacataaaatctgatggacaaacagagggttgctggggggacaagcgtgggcacaatttccccaccaagacaccctgatcttcaggcgggtctcaggagcttctaaaaatccgcatggctctcctgagagtggacagaggagaggagagggtcagaaatgaacgctcttctatttcttgtcattaccaagccaattacttttgccaaatttttctgtgatctgccctgattaagatgaattgtgaaatttacatcaagcaattatcaaagcgggctgggtcccatcagaacgacccacatctttctgtgggtgtgaatgtcattaggtcttgcgctgacccctgagcccccatcactgccgcctgatggggcaaagaaacaaaaaacatttcttactcttctgtgttttaacaaaagtttataaaacaaaataaatggcgcatatgttttctaaaaaaaaaaaaaaaaa

SEQ ID NO: 32 - DNA, 6460 bp, Homo sapiens
agatcttcccagaggacggtttgaaaggaaggcagagagggcactgggaggaggcagtgggagggcggagggcgggggccttcggggtgggcgcccagggtagggcaggtggccgcggcgtggaggcagggagaatgcgactctccaaaaccctcgtcgacatggacatggccgactacagtgctgcactggacccagcctacaccaccctggaatttgagaatgtgcaggtgttgacgatgggcaatgacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggagggtcccccagcgatgcaccccatgcccaccaccccctgcaccctcacctgatgcaggaacatatgggaaccaacgtcatcgttgccaacacaatgcccactcacctcagcaacggacagatgtccacccctgagaccccacagccctcaccgccaggtggctcagggtctgagccctataagctcctgccgggagccgtcgccacaatcgtcaagcccctctctgccatcccccagccgaccatcaccaagcaggaagttatctagcaagccgctggggcttgggggctccactggctccccccagccccctaagagagcacctggtgatcacgtggtcacggcaaaggaagacgtgatgccaggaccagtcccagagcaggaatgggaaggatgaagggcccgagaacatggcctaagggccacatcccactgccacccttgacgccctgctctggataacaagactttgacttggggagacctctactgccttggacaacttttctcatgttgaagccactgccttcaccttcaccttcatccatgtccaacccccgacttcatcccaaaggacagccgcctggagatgacttgaggccttacttaaacccagctcccttcttccctagcctggtgcttctcctctcctagcccctgtcatggtgtccagacagagccctgtgaggctgggtccaattgtggcacttggggcaccttgctcctccttctgctgctgcccccacctctgctgcctccctctgctgtcaccttgctcagccatcccgtcttctccaacaccacctctccagaggccaaggaggccttggaaacgattcccccagtcattctgggaacatgttgtaagcactgactgggaccaggcaccaggcagggtctagaaggctgtggtgagggaagacgcctttctcctccaacccaacctcatcctccttcttcagggacttgggtgggtacttgggtgaggatccctgaaggccttcaacccgagaaaacaaacccaggttggcgactgcaacaggaacttggagtggagaggaaaagcatcagaaagaggcagaccatccaccaggcctttgagaaagggtagaattctggctggtagagcaggtgagatgggacattccaaagaacagcctgagccaaggcctagtggtagtaagaatctagcaagaattgaggaagaatggtgtgggagagggatgatgaagagagagagggcctgctggagagcatagggtctggaacaccaggctgaggtcctgatcagcttcaaggagtatgcagggagctgggcttccagaaaatgaacacagcagttctgcagaggacgggaggctggaagctgggaggtcaggtggggtggatgatataatgcgggtgagagtaatgaggcttggggctggagaggacaagatgggtaaaccctcacatcagagtgacatccaggaggaataagctcccagggcctgtctcaagctcttccttactcccaggcactgtcttaaggcatctgacatgcatcatctcatttaatcctcccttcctccctattaacctagagattgtttttgttttttattctcctcctccctccccgccctcacccgccccactccctcctaacctagagattgttacagaagctgaaattgcgttctaagaggtgaagtgattttttttctgaaactcacacaactaggaagtggctgagtcaggacttgaacccaggtctccctggatcagaacaggagctcttaactacagtggctgaatagcttctccaaaggctccctgtgttctcaccgtgatcaagttgaggggcttccggctcccttctacagcctcagaaaccagactcgttcttctgggaaccctgcccactcccaggaccaagattggcctgaggctgcactaaaattcacttagggtcgagcatcctgtttgctgataaatattaaggagaattcatgactcttgacagcttttctctcttcactccccaagtcaaggggaggggtggcaggggtctgtttcctggaagtcaggctcatctggcctgttggcatgggggtgggacagtgtgcacagtgtgggggcaggggagggctaagcaggcctgggtttgagggctgctccggagaccgtcactccaggtgcattctggaagcattagaccccaggatggagcgaccagcatgtcatccatgtggaatcttggtggctttgaggacattctggaaaatgccactgaccagtgtgaacaaaagggatgtgttatggggctggaggtgtgattaggtaggagggaaactgttggaccgactcctgccccctgctcaacactgacccctctgagtggttggaggcagtgccccagtgcccagaaatcccaccattagtgattgttttttatgagaaagaggcgtggagaagtattggggcaatgtgtcagggaggaatcaccacatccctacggcagtcccagccaagcccccaatcccagcggagactgtgccctgctcagagctcccaagccttcccccaccacctcactcaagtgcccctgaaatccctgccagacggctcagcctggtctgcggtaaggcagggaggctggaaccatttctgggcattgtggtcattcccactgtgttcctccacctcctccctccagcgttgctcagacctctgtcttgggagaaaggttgagataagaatgtcccatggagtgccgtgggcaacagtggcccttcatgggaacaatctgttggagcagggggtcagttctctgctgggaatctacccctttctggaggagaaacccattccaccttaataactttattgtaatgtgagaaacacaaaacaaagtttacttttttgactctaagctgacatgatattagaaaatctctcgctctcttttttttttttttttttttttttggctacttgagttgtggtcctaaaacataaaatctgatggacaaacagagggttgctggggggacaagcgtgggcacaatttccccaccaagacaccctgatcttcaggcgggtctcaggagcttctaaaaatccgcatggctctcctgagagtggacagaggagaggagagggtcagaaatgaacgctcttctatttcttgtcattaccaagccaattacttttgccaaatttttctgtgatctgccctgattaagatgaattgtgaaatttacatcaagcaattatcaaagcgggctgggtcccatcagaacgacccacatctttctgtgggtgtgaatgtcattaggtcttgcgctgacccctgagcccccatcactgccgcctgatggggcaaagaaacaaaaaacatttcttactcttctgtgttttaacaaaagtttataaaacaaaataaatggcgcatatgttttctaagtccttggataagtatcttttctttcaggtatcagaaataagactgaatcttctggttctacttgggggttaaaaaattttttttaaaggaagaatgagaatagttttatagttctttgtgatgtgcagaatgtttttgtgtccattataatttttcagtcttcacatcaagaggtaagcagttagacatgattactcccactttccagatgaggagactgaggcttgggggaagtgacttctcttggaaggcagaggtggacatctaaccctggtctcttgattccaagtacttagtatatcgagagagtgaaagttgatcccccttcttgaagaggggagtgatgaggggagagtgcaatggcaagatctggaagaatggcaagagggtccaagggtctgtcatcctccaccaaggttcaagacagaaccttttgctgggtcacctcaatctgccagcaatggaagatgagtagctgtggggacatttcataaaagcaagtggtttttttgttttgttttgttttgttttttgtttttttttctagaacaaggctgtgcacagtggctcacctctgtaatcccagcactttgggaggctgaggcgggaggatcacttgagctcaggagttcgagaccagccagagcaatataaggagaccccatctctacaaaaaatttaaaaattagccaggtatagtggtgtgtgcctatagtaccagctactctgaaggctgaggtgggaagattgcttcagcccaggagttcgaggatgcagtgagctatgaatgcaacactgcactacagcctggatgaaagaacaagactctgtctcaaaacataaataataagtaaaaagaataaaagcaagagatgcacttgagaatctccagccagatctgtagccactgggcttctctccaaggctaaactattacaggagggtggccttgtgtctcggtcaccacagaccacagcgttccattcactcggggttgtgctggagctggcttgtgagaactgactgttagcttctcttcccaactccatgtttgccagtgccacactgatagcttgaaattggttattgccggagtgtttacaccacaaggactagcaaactctacaaatccgggcttttgttcctggagagcccgttgttaacattcaccagcacaccacagcattcggcaatggctggaccatgggatgcctacatatggggacatcctccttggggatgagggtagagcagggcgatcctttcacctcttccttaagggaggggacaaaagttctggtctgggaagcacacgttttgctgatcagcgtaaccttgggcaggtcactccaccactccgagcctcatctgtaaagtgggaatgatatctccctccagggcagatgtcaggattcaatggaatgagatcacagtaactgtgagagctcccgttacatgaggagtacaagtgaactcttcatgcgcccctttttagcgagaagttaaccattaaactctccaggcttcagagcacccattcgctgtctacctgatccctagggccgctcccgccttcccctgtgccttccctccactagtcagcaccaggaaatgttttcgataacgttgcaacggaggccttgttcatgctgccgccatcggggacaagcgcgggggggggggggtggaggccagaggagactatttcagtcctaaattgtgcttaataaacccatatcaaaaccataaa

SEQ ID NO: 33 - DNA, 6581 bp, Homo sapiens
agatcttcccagaggacggtttgaaaggaaggcagagagggcactgggaggaggcagtgggagggcggagggcgggggccttcggggtgggcgcccagggtagggcaggtggccgcggcgtggaggcagggagaatgcgactctccaaaaccctcgtcgacatggacatggccgactacagtgctgcactggacccagcctacaccaccctggaatttgagaatgtgcaggtgttgacgatgggcaatgattttgttgccgctgcgtctcgccagattgaggcatcccctccgacatcactggagcatatctggaggggtggacagttctccacagggagacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggagggtcccccagcgatgcaccccatgcccaccaccccctgcaccctcacctgatgcaggaacatatgggaaccaacgtcatcgttgccaacacaatgcccactcacctcagcaacggacagatgtgtgagtggccccgacccaggggacaggcagccacccctgagaccccacagccctcaccgccaggtggctcagggtctgagccctataagctcctgccgggagccgtcgccacaatcgtcaagcccctctctgccatcccccagccgaccatcaccaagcaggaagttatctagcaagccgctggggcttgggggctccactggctccccccagccccctaagagagcacctggtgatcacgtggtcacggcaaaggaagacgtgatgccaggaccagtcccagagcaggaatgggaaggatgaagggcccgagaacatggcctaagggccacatcccactgccacccttgacgccctgctctggataacaagactttgacttggggagacctctactgccttggacaacttttctcatgttgaagccactgccttcaccttcaccttcatccatgtccaacccccgacttcatcccaaaggacagccgcctggagatgacttgaggccttacttaaacccagctcccttcttccctagcctggtgcttctcctctcctagcccctgtcatggtgtccagacagagccctgtgaggctgggtccaattgtggcacttggggcaccttgctcctccttctgctgctgcccccacctctgctgcctccctctgctgtcaccttgctcagccatcccgtcttctccaacaccacctctccagaggccaaggaggccttggaaacgattcccccagtcattctgggaacatgttgtaagcactgactgggaccaggcaccaggcagggtctagaaggctgtggtgagggaagacgcctttctcctccaacccaacctcatcctccttcttcagggacttgggtgggtacttgggtgaggatccctgaaggccttcaacccgagaaaacaaacccaggttggcgactgcaacaggaacttggagtggagaggaaaagcatcagaaagaggcagaccatccaccaggcctttgagaaagggtagaattctggctggtagagcaggtgagatgggacattccaaagaacagcctgagccaaggcctagtggtagtaagaatctagcaagaattgaggaagaatggtgtgggagagggatgatgaagagagagagggcctgctggagagcatagggtctggaacaccaggctgaggtcctgatcagcttcaaggagtatgcagggagctgggcttccagaaaatgaacacagcagttctgcagaggacgggaggctggaagctgggaggtcaggtggggtggatgatataatgcgggtgagagtaatgaggcttggggctggagaggacaagatgggtaaaccctcacatcagagtgacatccaggaggaataagctcccagggcctgtctcaagctcttccttactcccaggcactgtcttaaggcatctgacatgcatcatctcatttaatcctcccttcctccctattaacctagagattgtttttgttttttattctcctcctccctccccgccctcacccgccccactccctcctaacctagagattgttacagaagctgaaattgcgttctaagaggtgaagtgattttttttctgaaactcacacaactaggaagtggctgagtcaggacttgaacccaggtctccctggatcagaacaggagctcttaactacagtggctgaatagcttctccaaaggctccctgtgttctcaccgtgatcaagttgaggggcttccggctcccttctacagcctcagaaaccagactcgttcttctgggaaccctgcccactcccaggaccaagattggcctgaggctgcactaaaattcacttagggtcgagcatcctgtttgctgataaatattaaggagaattcatgactcttgacagcttttctctcttcactccccaagtcaaggggaggggtggcaggggtctgtttcctggaagtcaggctcatctggcctgttggcatgggggtgggacagtgtgcacagtgtgggggcaggggagggctaagcaggcctgggtttgagggctgctccggagaccgtcactccaggtgcattctggaagcattagaccccaggatggagcgaccagcatgtcatccatgtggaatcttggtggctttgaggacattctggaaaatgccactgaccagtgtgaacaaaagggatgtgttatggggctggaggtgtgattaggtaggagggaaactgttggaccgactcctgccccctgctcaacactgacccctctgagtggttggaggcagtgccccagtgcccagaaatcccaccattagtgattgttttttatgagaaagaggcgtggagaagtattggggcaatgtgtcagggaggaatcaccacatccctacggcagtcccagccaagcccccaatcccagcggagactgtgccctgctcagagctcccaagccttcccccaccacctcactcaagtgcccctgaaatccctgccagacggctcagcctggtctgcggtaaggcagggaggctggaaccatttctgggcattgtggtcattcccactgtgttcctccacctcctccctccagcgttgctcagacctctgtcttgggagaaaggttgagataagaatgtcccatggagtgccgtgggcaacagtggcccttcatgggaacaatctgttggagcagggggtcagttctctgctgggaatctacccctttctggaggagaaacccattccaccttaataactttattgtaatgtgagaaacacaaaacaaagtttacttttttgactctaagctgacatgatattagaaaatctctcgctctcttttttttttttttttttttttttggctacttgagttgtggtcctaaaacataaaatctgatggacaaacagagggttgctggggggacaagcgtgggcacaatttccccaccaagacaccctgatcttcaggcgggtctcaggagcttctaaaaatccgcatggctctcctgagagtggacagaggagaggagagggtcagaaatgaacgctcttctatttcttgtcattaccaagccaattacttttgccaaatttttctgtgatctgccctgattaagatgaattgtgaaatttacatcaagcaattatcaaagcgggctgggtcccatcagaacgacccacatctttctgtgggtgtgaatgtcattaggtcttgcgctgacccctgagcccccatcactgccgcctgatggggcaaagaaacaaaaaacatttcttactcttctgtgttttaacaaaagtttataaaacaaaataaatggcgcatatgttttctaagtccttggataagtatcttttctttcaggtatcagaaataagactgaatcttctggttctacttgggggttaaaaaattttttttaaaggaagaatgagaatagttttatagttctttgtgatgtgcagaatgtttttgtgtccattataatttttcagtcttcacatcaagaggtaagcagttagacatgattactcccactttccagatgaggagactgaggcttgggggaagtgacttctcttggaaggcagaggtggacatctaaccctggtctcttgattccaagtacttagtatatcgagagagtgaaagttgatcccccttcttgaagaggggagtgatgaggggagagtgcaatggcaagatctggaagaatggcaagagggtccaagggtctgtcatcctccaccaaggttcaagacagaaccttttgctgggtcacctcaatctgccagcaatggaagatgagtagctgtggggacatttcataaaagcaagtggtttttttgttttgttttgttttgttttttgtttttttttctagaacaaggctgtgcacagtggctcacctctgtaatcccagcactttgggaggctgaggcgggaggatcacttgagctcaggagttcgagaccagccagagcaatataaggagaccccatctctacaaaaaatttaaaaattagccaggtatagtggtgtgtgcctatagtaccagctactctgaaggctgaggtgggaagattgcttcagcccaggagttcgaggatgcagtgagctatgaatgcaacactgcactacagcctggatgaaagaacaagactctgtctcaaaacataaataataagtaaaaagaataaaagcaagagatgcacttgagaatctccagccagatctgtagccactgggcttctctccaaggctaaactattacaggagggtggccttgtgtctcggtcaccacagaccacagcgttccattcactcggggttgtgctggagctggcttgtgagaactgactgttagcttctcttcccaactccatgtttgccagtgccacactgatagcttgaaattggttattgccggagtgtttacaccacaaggactagcaaactctacaaatccgggcttttgttcctggagagcccgttgttaacattcaccagcacaccacagcattcggcaatggctggaccatgggatgcctacatatggggacatcctccttggggatgagggtagagcagggcgatcctttcacctcttccttaagggaggggacaaaagttctggtctgggaagcacacgttttgctgatcagcgtaaccttgggcaggtcactccaccactccgagcctcatctgtaaagtgggaatgatatctccctccagggcagatgtcaggattcaatggaatgagatcacagtaactgtgagagctcccgttacatgaggagtacaagtgaactcttcatgcgcccctttttagcgagaagttaaccattaaactctccaggcttcagagcacccattcgctgtctacctgatccctagggccgctcccgccttcccctgtgccttccctccactagtcagcaccaggaaatgttttcgataacgttgcaacggaggccttgttcatgctgccgccatcggggacaagcgcgggggggggggggtggaggccagaggagactatttcagtcctaaattgtgcttaataaacccatatcaaaaccataaa

SEQ ID NO: 34 - DNA, 6588 bp, Homo sapiens
gcactcaccgccttcctggtggacgggctcctggtggctgtgctgctgctgtgagcgggcccctgctcctccatgcccccagctctccggctgggtgggcttggccatggtcagcgtgaacgcgcccctcggggctccagtggagagttcttacggtctgcagtttccatggagaccgcctcccaccatccctgaaatctcattgtgaccagtattaagggattaactctgcccagcggccctcgcagggtctggggtgctgagaggggccagcaggagctccctcaaggatgaagaggttgtgcactgtgcgggaccccatagcagcaggaggaggatgtcggactggggccagggcttcccccaggacccaccagacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggagggtcccccagcgatgcaccccatgcccaccaccccctgcaccctcacctgatgcaggaacatatgggaaccaacgtcatcgttgccaacacaatgcccactcacctcagcaacggacagatgtccacccctgagaccccacagccctcaccgccaggtggctcagggtctgagccctataagctcctgccgggagccgtcgccacaatcgtcaagcccctctctgccatcccccagccgaccatcaccaagcaggaagttatctagcaagccgctggggcttgggggctccactggctccccccagccccctaagagagcacctggtgatcacgtggtcacggcaaaggaagacgtgatgccaggaccagtcccagagcaggaatgggaaggatgaagggcccgagaacatggcctaagggccacatcccactgccacccttgacgccctgctctggataacaagactttgacttggggagacctctactgccttggacaacttttctcatgttgaagccactgccttcaccttcaccttcatccatgtccaacccccgacttcatcccaaaggacagccgcctggagatgacttgaggccttacttaaacccagctcccttcttccctagcctggtgcttctcctctcctagcccctgtcatggtgtccagacagagccctgtgaggctgggtccaattgtggcacttggggcaccttgctcctccttctgctgctgcccccacctctgctgcctccctctgctgtcaccttgctcagccatcccgtcttctccaacaccacctctccagaggccaaggaggccttggaaacgattcccccagtcattctgggaacatgttgtaagcactgactgggaccaggcaccaggcagggtctagaaggctgtggtgagggaagacgcctttctcctccaacccaacctcatcctccttcttcagggacttgggtgggtacttgggtgaggatccctgaaggccttcaacccgagaaaacaaacccaggttggcgactgcaacaggaacttggagtggagaggaaaagcatcagaaagaggcagaccatccaccaggcctttgagaaagggtagaattctggctggtagagcaggtgagatgggacattccaaagaacagcctgagccaaggcctagtggtagtaagaatctagcaagaattgaggaagaatggtgtgggagagggatgatgaagagagagagggcctgctggagagcatagggtctggaacaccaggctgaggtcctgatcagcttcaaggagtatgcagggagctgggcttccagaaaatgaacacagcagttctgcagaggacgggaggctggaagctgggaggtcaggtggggtggatgatataatgcgggtgagagtaatgaggcttggggctggagaggacaagatgggtaaaccctcacatcagagtgacatccaggaggaataagctcccagggcctgtctcaagctcttccttactcccaggcactgtcttaaggcatctgacatgcatcatctcatttaatcctcccttcctccctattaacctagagattgtttttgttttttattctcctcctccctccccgccctcacccgccccactccctcctaacctagagattgttacagaagctgaaattgcgttctaagaggtgaagtgattttttttctgaaactcacacaactaggaagtggctgagtcaggacttgaacccaggtctccctggatcagaacaggagctcttaactacagtggctgaatagcttctccaaaggctccctgtgttctcaccgtgatcaagttgaggggcttccggctcccttctacagcctcagaaaccagactcgttcttctgggaaccctgcccactcccaggaccaagattggcctgaggctgcactaaaattcacttagggtcgagcatcctgtttgctgataaatattaaggagaattcatgactcttgacagcttttctctcttcactccccaagtcaaggggaggggtggcaggggtctgtttcctggaagtcaggctcatctggcctgttggcatgggggtgggacagtgtgcacagtgtgggggcaggggagggctaagcaggcctgggtttgagggctgctccggagaccgtcactccaggtgcattctggaagcattagaccccaggatggagcgaccagcatgtcatccatgtggaatcttggtggctttgaggacattctggaaaatgccactgaccagtgtgaacaaaagggatgtgttatggggctggaggtgtgattaggtaggagggaaactgttggaccgactcctgccccctgctcaacactgacccctctgagtggttggaggcagtgccccagtgcccagaaatcccaccattagtgattgttttttatgagaaagaggcgtggagaagtattggggcaatgtgtcagggaggaatcaccacatccctacggcagtcccagccaagcccccaatcccagcggagactgtgccctgctcagagctcccaagccttcccccaccacctcactcaagtgcccctgaaatccctgccagacggctcagcctggtctgcggtaaggcagggaggctggaaccatttctgggcattgtggtcattcccactgtgttcctccacctcctccctccagcgttgctcagacctctgtcttgggagaaaggttgagataagaatgtcccatggagtgccgtgggcaacagtggcccttcatgggaacaatctgttggagcagggggtcagttctctgctgggaatctacccctttctggaggagaaacccattccaccttaataactttattgtaatgtgagaaacacaaaacaaagtttacttttttgactctaagctgacatgatattagaaaatctctcgctctcttttttttttttttttttttttttggctacttgagttgtggtcctaaaacataaaatctgatggacaaacagagggttgctggggggacaagcgtgggcacaatttccccaccaagacaccctgatcttcaggcgggtctcaggagcttctaaaaatccgcatggctctcctgagagtggacagaggagaggagagggtcagaaatgaacgctcttctatttcttgtcattaccaagccaattacttttgccaaatttttctgtgatctgccctgattaagatgaattgtgaaatttacatcaagcaattatcaaagcgggctgggtcccatcagaacgacccacatctttctgtgggtgtgaatgtcattaggtcttgcgctgacccctgagcccccatcactgccgcctgatggggcaaagaaacaaaaaacatttcttactcttctgtgttttaacaaaagtttataaaacaaaataaatggcgcatatgttttctaagtccttggataagtatcttttctttcaggtatcagaaataagactgaatcttctggttctacttgggggttaaaaaattttttttaaaggaagaatgagaatagttttatagttctttgtgatgtgcagaatgtttttgtgtccattataatttttcagtcttcacatcaagaggtaagcagttagacatgattactcccactttccagatgaggagactgaggcttgggggaagtgacttctcttggaaggcagaggtggacatctaaccctggtctcttgattccaagtacttagtatatcgagagagtgaaagttgatcccccttcttgaagaggggagtgatgaggggagagtgcaatggcaagatctggaagaatggcaagagggtccaagggtctgtcatcctccaccaaggttcaagacagaaccttttgctgggtcacctcaatctgccagcaatggaagatgagtagctgtggggacatttcataaaagcaagtggtttttttgttttgttttgttttgttttttgtttttttttctagaacaaggctgtgcacagtggctcacctctgtaatcccagcactttgggaggctgaggcgggaggatcacttgagctcaggagttcgagaccagccagagcaatataaggagaccccatctctacaaaaaatttaaaaattagccaggtatagtggtgtgtgcctatagtaccagctactctgaaggctgaggtgggaagattgcttcagcccaggagttcgaggatgcagtgagctatgaatgcaacactgcactacagcctggatgaaagaacaagactctgtctcaaaacataaataataagtaaaaagaataaaagcaagagatgcacttgagaatctccagccagatctgtagccactgggcttctctccaaggctaaactattacaggagggtggccttgtgtctcggtcaccacagaccacagcgttccattcactcggggttgtgctggagctggcttgtgagaactgactgttagcttctcttcccaactccatgtttgccagtgccacactgatagcttgaaattggttattgccggagtgtttacaccacaaggactagcaaactctacaaatccgggcttttgttcctggagagcccgttgttaacattcaccagcacaccacagcattcggcaatggctggaccatgggatgcctacatatggggacatcctccttggggatgagggtagagcagggcgatcctttcacctcttccttaagggaggggacaaaagttctggtctgggaagcacacgttttgctgatcagcgtaaccttgggcaggtcactccaccactccgagcctcatctgtaaagtgggaatgatatctccctccagggcagatgtcaggattcaatggaatgagatcacagtaactgtgagagctcccgttacatgaggagtacaagtgaactcttcatgcgcccctttttagcgagaagttaaccattaaactctccaggcttcagagcacccattcgctgtctacctgatccctagggccgctcccgccttcccctgtgccttccctccactagtcagcaccaggaaatgttttcgataacgttgcaacggaggccttgttcatgctgccgccatcggggacaagcgcgggggggggggggtggaggccagaggagactatttcagtcctaaattgtgcttaataaacccatatcaaaaccataaa